(19) United States
(12) Patent Application Publication
(10) Pub. No.: US 2024/0139187 A1
(43) Pub. Date: May 2, 2024

(54) SELECTIVE HYPOTHALAMUS PERMEABLE HDAC6 INHIBITORS FOR TREATMENT OF LEPTIN-RESISTANT OBESITY

(71) Applicants: The Children's Medical Center Corporation, Boston, MA (US); The General Hospital Corporation, Boston, MA (US)

(72) Inventors: Umut Ozcan, Weston, MA (US); Ralph Mazitschek, Weston, MA (US); Dongxian Guan, Brighton, MA (US)

(21) Appl. No.: 18/324,598

(22) Filed: May 26, 2023

Related U.S. Application Data
(60) Provisional application No. 63/379,215, filed on Oct. 12, 2022.

(51) Int. Cl. A61K 31/506; A61K 31/195; A61K 31/505; A61P 3/04

(52) U.S. Cl. CPC A61K 31/506; A61K 31/195; A61K 31/505; A61P 3/04

(57) ABSTRACT

Formulations of HDAC6 inhibitors passing through the blood brain barrier in hypothalamus and inhibiting HDAC6 in the arctuate AgRP neurons in the hypothalamus, are effective to cause weight loss in obese individuals. These inhibitors also restore leptin sensitivity in leptin-resistant individuals.

### CROSS-REFERENCE TO RELATED APPLICATIONS

This application claims the benefit of and priority to U.S. Provisional Application No. 63/379,215 filed Oct. 12, 2022, which is hereby incorporated by reference in its entirety.

### STATEMENT REGARDING FEDERALLY SPONSORED RESEARCH OR DEVELOPMENT

This invention was made with government support under Grant No. R01 DK098496-02 awarded by the National Institutes of Health. The government has certain rights in the invention.

### FIELD OF THE INVENTION

This is generally in the field of treatments for obesity, especially leptin-resistant obesity.

### BACKGROUND OF THE INVENTION

Since 1975, worldwide obesity has nearly tripled and currently contributes to more than 5 million deaths each year. The disease, which is defined as “abnormal or excessive fat accumulation that presents a risk to health,” affects one of every three adults, or about 36% of the population, including 39 million children under the age of 5 in 2020.

Obesity is a global epidemic that can have dire health consequences, as well as increased costs that have the potential to paralyze the healthcare system. The most obvious direct costs of obesity are related to co-morbid conditions and the medications needed to treat these diseases. Currently, there are 236 diseases that are associated with obesity including type 2 diabetes, hypertension, and depression. Recent data found that the medical care costs of obesity are almost $150 billion per year in the U.S. Obesity-associated co-morbidities such as hypertension, dyslipidemia, type 2 diabetes mellitus, fatty liver disease, heart disease, and some types of cancer cause about 3.4 million adults (over age 18) deaths in 2016, according to the World Health Organization. As reported, 1.9 billion adults are overweight, and over 650 million overweight adults are obese. Hyperleptinemia and resistance to a reduction of body mass are two common characteristics of obesity.

The peptide hormone leptin regulates food intake, body mass, and reproductive function and plays a role in fetal growth, proinflammatory immune responses, angiogenesis and lipolysis. Leptin is a product of the obese (ob) gene and, following synthesis and secretion from fat cells in white adipose tissue, binds to and activates its cognate receptor, the leptin receptor (LEP-R). LEP-R distribution facilitates leptin's pleiotropic effects, playing a crucial role in regulating body mass via a negative feedback mechanism between adipose tissue and the hypothalamus. The leptin receptor receives and transmits signals from leptin, a hormone released from fat cells that is involved primarily in the regulation of metabolism but also serves roles in bone metabolism, immunity, and reproductive function. The leptin receptor is located in the cell membrane in various tissues in the body but is most highly expressed on neurons in the hypothalamus, a region of the brain involved in regulating hunger, body temperature, sleep, and other activities. It is a member of a superfamily of cytokine receptor proteins. When leptin binds to leptin receptors, specifically in the hypothalamus, chemical signals are produced that promote a feeling of satiety, reducing hunger. The significance of this function is apparent in the case of leptin receptor deficiency, which is associated with elevated blood plasma levels of leptin and, consequently, persistent hunger and overeating, leading to obesity.

Leptin resistance is characterized by reduced satiety, over-consumption of nutrients, and increased total body mass. There is a strong positive association between serum leptin levels and the percentage of body fat. Leptin regulates food intake, body mass, reproductive functioning and plays a vital role in fetal growth, proinflammatory immune responses, angiogenesis, and lipolysis.

Brain lesion and stimulation research led to the discovery of the “satiety center” in the ventromedial hypothalamic nucleus (VMH) and the “hunger center” in the lateral hypothalamic nuclei (LH). This defines the dual-center model for feeding. Leptin regulates appetite and metabolism mainly through acting on a subset of neurons expressing AgRP in the arcuate nucleus (ARC). Depletion of LepR in these neurons almost completely abolishes leptin's appetite suppressing and bodyweight lowering effect. Thus, AgRP-expressing neurons' LepR is essential to mediate leptin's effect. Leptin can inhibit neural pathways activated by appetite stimulants (orexigenic) to reduce energy intake and activate pathways targeted by anorexigenic to suppress appetite.

Hyperleptinemia and resistance to reducing body mass are two characteristics of typical obesity. Leptin is overexpressed at the gene level in the adipose tissue of individuals with obesity. Furthermore, strong positive associations exist between plasma leptin levels and body fat percentage. Other studies point towards leptin resistance. For example, plasma leptin levels and ob mRNA content decrease in individuals with obesity at the initial time of weight loss but increases as they continue to lose weight.

A number of compounds have been proposed to treat obesity, including leptin-resistant obesity, but with mixed results.

Cakir, et al. Nat Metab. 2022 January; 4(1): 44-59. doi:10.1038/s42255-021-00515-3, reported that inhibitors of the cytosolic enzyme histone deacetylase 6 (HDAC6) act as potent leptin sensitizers and anti-obesity agents in diet-induced obese mice. Specifically, HDAC6 inhibitors, such as tubastatin A, reduce food intake, fat mass, hepatic steatosis and improve systemic glucose homeostasis in an HDAC6-dependent manner. Data show that peripheral, but not central, inhibition of HDAC6 confers central leptin sensitivity. Additionally, the anti-obesity effect of tubastatin A is attenuated in animals with a defective central leptin—melanocortin circuitry, including db/db and MC4R knockout mice, indicating that an HDAC6-regulated adipokine serves as a leptin-sensitizing agent and reveals HDAC6 as a potential target for the treatment of obesity. HDAC6 inhibitors that were tested included tubastatin and an inactive analog thereof, BRD3067, and selective HDAC6 inhibitors CAY10603, and ricolinostat. It was alleged that tubastatin reverses leptin resistance in mice via a peripheral mechanism of action, perhaps by inducing release of a leptin-sensitizing factor. Levels of tubastatin in the brain were extremely low due to low penetration of the blood brain barrier (“BBB”). They concluded that tubastatin acted via a dual mode-of-action for tubastatin-induced weight loss, in which peripheral inhibition HDAC6 activity by a selective HDAC6 inhibitor leads to a release of systemic factor from periphery that acts in the CNS to increase leptin sensitivity.

Obesity is an ongoing and difficult problem to treat, especially leptin-resistant obesity. There remains a significant need for compounds to safely effect weight loss, especially leptin-resistant obesity.

It is therefore an object of the present invention to provide compounds and methods of use thereof for the treatment of obesity, especially leptin-resistant obesity, where the compounds pass through the blood brain barrier and act on central mechanisms of action.

### SUMMARY OF THE INVENTION

It has been discovered that HDAC6 inhibitors with high levels of penetration of the BBB, especially the hypothalamus, especially inhibitors which block LepRb-HDAC6 interaction in the AgRP neurons in the hypothalamus, are effective in treating leptin-resistant obesity, as well as to increase leptin sensitivity. It has been further discovered that the critical region of the brain for the inhibitors to be effective is the hypothalamus. Several compounds were tested and it was determined that a central, not peripheral, mechanism of action was involved with the HDAC6 inhibitors that had high levels of penetration through the blood brain barrier and into the hypothalamus. Analysis of hypothalamic gene expressions in fed, starved and obese mice identified correlations with gene expression changes induced by small molecule inhibition of HDAC6 activity, establishing that pharmacological inhibition of HDAC6 activity in the hypothalamus is effective for the treatment of obesity and related disorders. Subsequent studies in high fat diet induced mice treated with HDAC6 isoform-specific inhibitors, ACY738 and ACY775, showed these compounds caused significant reduction of food intake and total body fat mass. Effects were greater for the more selective HDAC6 inhibitor ACY775 as compared to ACY738. Significant weight loss was also demonstrated by bavarostat and a compound that inhibits HDAC6 in the hypothalamus but has low BBB penetration, ACY257, and ACY1083.

Only HDAC6 inhibitors which penetrate the hypothalamus and inhibit HDAC6 therein are effective to treat obesity, especially leptin-resistant obesity.

Data demonstrates that a compound, ACY 257, which inhibits HDAC6 in the hypothalamus but does not inhibit HDAC6 in other brain regions such as cerebrum, frontal lobe, temporal lobe, and brainstem, greatly reduces the body weight. The hypothalmus is a very small part of the brain. The hypothalamus has a slightly more permeable blood brain barrier compared to other parts of the brain. The data indicates that inhibitors of HDAC6 which have the ability to preferentially pass through the BBB and into the hypothalamic region, especially the arcuate nucleus, are able to reduce body weight.

Based on these studies, it was determined that small molecule (less than 1000 Da) selective inhibitors of HDAC6 (inhibitors result in a-tubulin acetylation with no impact on histone acetylation), especially inhibitors which block LepRb-HDAC6 interaction, having a high penetration through the BBB of greater than 1 brain/plasma ratio and of the hypothalamus, are most effective for treatment of obesity, including leptin-resistant obesity, via a central nervous system (CNS) mechanism of action. Preferred HDAC6 inhibitors have >0.25, >0.5, or >1 brain or hypothalamus/plasma concentration. More preferably, the HDAC6 inhibitors have >0.25, >0.5, or >1 hypothalamus/plasma concentration. Most preferably, HDAC6 inhibitors which have >0.25, >0.5, or >1 Arcuate Nucleus/plasma concentration. The arcuate nucleus has the highest BBB permeability in the hypothalamus and is around 1/10,000 of the total area of the brain. The results indicate that inhibiting HDAC6 in the AgRP neurons in the arcuate nucleus is sufficient to create the weight loss. If an HDAC6 inhibitor just gets to the arcuate nucleus at concentrations that can inhibit HDAC6 activity, but not to the other regions of the brain, total brain/plasma or hypothalamus/plasma concentrations may be low. However, the arcuate nucleus concentration will be higher than total brain/plasma and hypothalamus/plasma concentrations.

Preferred compounds are ACY775 (brain/plasma ratio 1.26) and ACY738 (brain/plasma ratio of 1.22), ACY257 which has high penetrance into the arctuate nucleus of the hypothalamus, ACY1083, and bavarostat (4-(((((3r,5r,7r)-adamantan-1-yl)methyl)(methyl)amino)methyl)-3-fluoro-N-hydroxybenzamide), which has high penetrance of the hypothalamus. Peripherally acting HDAC6 inhibitors such as ricolinostat (ACY1215) with a brain/plasma ratio of 0.01 and tubastatin A with a brain/plasma ratio of 0.18, neither of which have high penetrance into the hypothalamus nor citarinostat (ACY241), which does not show HDAC6 inhibition in the AGP neurons in the hypothalamus, are effective. HDAC6 inhibition was tested by treating cells with a high concentration of HDAC6 inhibitor and analyzing for tubulin acetylation, the established biomarker for HDAC6 inhibition.

The selective HDAC6 inhibitors are preferably administered to a mucosal surface, most preferably orally, buccally, or nasally. These can be formulated using known excipients. Formulations may also be formulated for sustained, delayed, and/or pulsatile release to deliver an effective amount of HDAC6 inhibitor to cause weight loss. They are preferably administered once or twice daily. Dosage is based on weight. Typical dosages will be in the range of 25 to 500 mg/day.

The pharmaceutical formulations can be administered to induce weight loss in a pre-obese, obese, or morbidly obese patient, reduce body fat in a pre-obese, obese, or morbidly obese patient, reduce food intake in a pre-obese, obese, or morbidly obese patient, improve glucose homeostasis in a pre-obese, obese, or morbidly obese patient, or combinations thereof. In some cases, a pharmaceutical formulation containing one or more of the HDAC6 inhibitors is administered to a pre-obese, obese, or morbidly obese patient in a therapeutically effective amount to induce weight loss, preferably in a therapeutically effective amount and time of administration to decrease body mass or body fat by at least 10%, more preferably by at least 15%, most preferably by at least 20%, or higher. In some cases, a pharmaceutical formulation containing one or more of the HDAC6 inhibitors is administered to a pre-obese, obese, or morbidly obese patient in a therapeutically effective amount to reduce food intake, appetite, or combinations thereof, preferably in a therapeutically effective amount to reduce average daily food intake (in terms of calories). In some cases, a pharmaceutical formulation containing one or more of the selective HDAC6 inhibitors is administered to a pre-obese, obese, or morbidly obese patient in a therapeutically effective amount to improve glucose homeostasis, preferably in a therapeutically effective amount to reduce average fasting plasma blood glucose. In cases where the pharmaceutical formulations are administered to normalize blood sugar, the formulations are preferably administered in an amount effective to lower blood glucose levels to less than about 180 mg/dL. The formulations can be co-administered with other anti-diabetic therapies, if necessary, to improve glucose homeostasis.

### BRIEF DESCRIPTION OF THE DRAWINGS

FIG. 1 is a graph of p-stat3/stat3 over time in minutes for control and HDAC6-OE.

FIGS. 2A-2O show that specific HDAC6 inhibitor (ACY775) increase leptin sensitivity (2C), decreases appetite (FIGS. 2B, 2E) and bodyweight. DIO mice (FIG. 2D) were pretreated with ACY775 (10mg/kg/day, i.p.) or vehicle for 3 days, which was followed by co-treatment with ACY775 (or vehicle) and leptin (1mg/kg, i.p.) (or saline) (n=9 for each group). FIGS. 2A, 2D, % change in body weight 15 h after leptin treatment, FIG. 2B, food intake (g) during the 15 h period after leptin treatment. The results were reproduced in two independent experiments. FIGS. 2C, 2E, DIO mice were pretreated with ACY775 (10mg/kg/day, i.p.) or vehicle for 3 days, which was followed by a last injection of ACY775 (or vehicle) in the morning of fourth day. 6 hours later mice were injected with saline or leptin (1 mg/kg, i.p.) and hypothalamus were extracted after 45 minutes following the leptin injection. Quantification of the ratio of p-STAT3^{Tyr705} to total-STAT3 signal in the immunoblots. The results were reproduced in three independent experiments. DIO mice were injected with HDAC6 inhibitors (ACY775, 10 mg/kg, i.p. or ACY738, 50 mg/kg, i.p., n=3 until 3:00 am. DIO mice were treated with ACY775 (10 mg/kg/day, i.p.) for 3 weeks. Daily body weight (g) of DIO mice treated with vehicle (n=17) or ACY775 (n=17) during 3 weeks of treatment. FIG. 2F-2O: FIG. 2F. 24 hours' food intake per mouse during the first week of vehicle or ACY775 treatment. FIG. 2G, Serum leptin (ng/ml) levels after 3 weeks of vehicle (n=24) or ACY775 (n=24) treatment. FIG. 2H, lean mass (g), FIG. 2I, fat mass (g), and FIG. 2J, fat percentage (%) after 3 weeks of vehicle (n=17) or ACY775(n=17) treatment measured by Dual-energy X-ray absorptiometry (DEXA) scan. DEXA scans were performed in two different cohorts. FIG. 2K, Glucose tolerance test (GTT) over 120 minutes after 1 week of vehicle (n=33) or ACY775 (n=26) treated DIO mice. FIG. 2L, Area of curve (AOC) analysis for GTT performed in (FIG. 2K). FIG. 2M, Blood glucose (mg/dl) levels over 120 minutes; FIG. 2O, after 1 week of vehicle (n=17) or ACY775 (n=16) treated DIO mice. FIG. 2N, Insulin tolerance test (ITT) after 2 weeks of vehicle (n=31) or ACY775 (n=21) treated DIO mice. q, Serum insulin (ng/ml) levels after 3 weeks of vehicle (n=22) or ACY775 (n=21) treated DIO mice. r, Homeostatic model assessment of insulin resistance (HOMA-IR) analysis after 3 weeks of vehicle (n=14) or ACY775 (n=13) treated DIO mice. s, H&E staining of the liver sections from 3 weeks of vehicle or ACY775 treated DIO mice. The results were reproduced in two independent experiments. Values indicate averages±s.e.m. P values were determined by two-way ANOVA with Bonferroni's multiple-comparisons test for curve (FIGS. 2D, 2E, 2K, 2M) or student' T-test for two groups' comparison (FIGS. 2A, 2B, 2C, 2F-2J, 2L, 2O, 2N). *p<0.05, **p<0.01, ***p<0.001, ****p<0.0001; n.s., not significant (p>0.05).

FIGS. 3A-3Q. Specific HDAC6 inhibitor (ACY738) increase leptin sensitivity, decreases appetite and bodyweight. (FIGS. 3A-3B) DIO mice were pretreated with ACY738 (50 mg/kg/day, i.p.) or vehicle for 3 days, which was followed by co-treatment with ACY738 (or vehicle) and leptin (1 mg/kg, i.p.) (or saline) (n=14 for each group). FIG. 3A, % change in body weight 15 h after leptin treatment, FIG. 3B, food intake (g) during the 15 h period after leptin treatment. The results in (FIGS. 3A and 3B) were reproduced in two independent experiments.) DIO mice were pretreated with ACY738 (50 mg/kg/day, i.p.) or vehicle 3 days, which was followed by a last injection of ACY738 (or vehicle) in the morning of fourth day. 6 hours later mice were injected with saline or leptin (1 mg/kg, i.p.) and hypothalamus were extracted after 45 minutes following the leptin injection. FIG. 3C, Quantification of the ratio of p-STAT3^{Tyr705} to total-STAT3 signal in the immunoblots. The result in (FIG. 3C) was reproduced in two independent experiments. FIG. 3D, DIO mice were injected with HDAC6 inhibitors (ACY775, 10 mg/kg, i.p. or ACY738, 50 mg/kg, i.p., n=3 cages with 3 mouse/cage for each group) after 24 h fasting. Food intake was analyzed hourly until 3:00 am. (FIG. 3E-3Q), DIO mice were treated with ACY738 (50 mg/kg, i.p.) for 3 weeks. FIG. 3E, Daily body weight (g) of DIO mice treated with vehicle (n=27) or ACY738 (n=24) during the 3 weeks of treatment period. FIG. 3F, Daily food intake (g) during 3 weeks of vehicle or ACY738 treatment. FIG. 3G, 24 hours' food intake per mouse during the first week of vehicle or ACY738 treatment. FIG. 3H, Serum leptin (ng/ml) levels after 3 weeks of vehicle (n=26) or ACY738 (n=25) treatment. FIG. 3I, lean mass (g) FIG. 3J, fat mass (g) and FIG. 3K, fat percentage (%) after 3 weeks of vehicle (n=23) or ACY738(n=23) treatment measured by DEXA scan. DEXA scans were performed in two different cohorts. FIG. 3L, GTT over 120 minutes; FIG. 3M is GTT after 1 week of vehicle (n=23) or ACY738 (n=24) treated DIO mice. FIG. 3N, AOC analysis for GTT performed in (FIG. 3L). FIG. 3O, Blood glucose (mg/dl) levels after 1 week of vehicle (n=27) or ACY738 (n=23) treated DIO mice. FIG. 3P, ITT after 2 weeks of vehicle (n=27) or ACY738 (n=22) treated DIO mice. FIG. 3Q, Serum insulin (ng/ml) level after 3 weeks of vehicle (n=25) or ACY738 (n=25) treated DIO mice. The results in (FIGS. 3E-3Q) were reproduced in two independent experiments. Values indicate averages±s.e.m. P values were determined by two-way ANOVA with Bonferroni's multiple-comparisons test for curve (FIGS. 3D, 3E, 3F, 3L, 3O) or student' T-test for two groups' comparison (FIGS. 3A, 3B, 3C, 3G-3K, 3M, 3N, 3P, 3Q). *p<0.05, **p<0.01, ***p<0.001, ****p<0.0001; n.s., not significant (p>0.05).

FIGS. 4A-4K. HDAC6 Inhibitor (ACY775) decreases appetite in DIO mice. Conditioned place preference (CPP) assay under the ad libitum feeding condition: DIO mice were treated with vehicle or ACY775 (10 mg/kg, i.p., once a day) for 3 days. On the fourth day, 1 h after the beginning of the light cycle, the mice were administered vehicle or ACY775 (10 mg/kg, i.p.) and the CPP assay was performed 6 h later under the ad libitum feeding condition (n=10 mice for both the Veh- and ACY775-treated groups). CPP assay under a 20-h fasting condition: DIO mice were treated with vehicle or ACY775 (10 mg/kg, i.p.) once a day and fasted for 15 h after the third injection. On the fourth day, 1 h after the beginning of the light cycle, the mice were administered vehicle or ACY775 (10 mg/kg body weight, i.p.) and the CPP assay was performed 5 h after this injection under the fasting condition (n=10 mice for both the Veh- and ACY775-treated groups). FIG. 4A, Total distance that the mice traveled during the CPP test. FIG. 4B, Average velocity of movement during the assay. FIG. 4C, Total time the mice spent in the dark chamber during the test. FIG. 4D, Frequency with which the mice traveled to the dark chamber during the test. FIG. 4E, Total time the mice spent in the food-paired white chamber. FIG. 4F, Frequency with which the mice traveled to the food paired white chamber. FIG. 4G, Total time the mice spent in the food-containing zone within the food-paired side chamber. FIG. 4H, Frequency with which the mice traveled to the food-containing zone within the food-paired side chamber during the test. Values indicate averages±s.e.m. P values were determined by student' T-test for two groups' comparison in this study (FIGS. 4A-4H). *p<0.05, **p<0.01, ***p<0.001, ****p<0.0001; n.s., not significant (p>0.05). FIGS. 4I-4K, Energy expenditure (FIG. 41) and respiratory exchange ratios (RER; VCO2/VO2) (FIG. 4J) in DIO mice treated with ACY738 (50 mg/kg, i.p., n=7) or veh (n=8) once a day for 3 day. Similar with ACY775, RER values were significantly decreased in ACY738-treated DIO mice in both the dark and light cycles, indicating more utilization of fat as energy source in the body. FIG. 4K, Physical activity level during the dark and light cycles were not different between ACY738- and vehicle-treated groups).

FIGS. 5A-5H. HDAC6 inhibitors do not reduce appetite or body weight in lean mice. WT lean mice were treated with vehicle (n=20) or ACY775 (10mg/kg, i.p., once a day, n=22) or ACY738 (50 mg/kg, i.p., once a day, n=20) for 3 weeks. FIG. 5A, Daily body weight (g) of WT lean mice treated with Veh, or ACY775 or ACY738 during 3 weeks of treatment period. FIG. 5B, Daily food intake (g) during 3 weeks of treatment. FIG. 5C, Serum leptin (ng/ml) levels after 3 weeks of treatment. FIG. 5D, lean mass (g) FIG. 5E, fat mass (g) and FIG. 5F, fat percentage (%) after 3 weeks of treatment measured by DEXA scan. FIG. 5G, GTT after 1 week of treatment. FIG. 5H,

FIGS. 5I-5Q and 5U-5W. HDAC6 inhibitors are ineffective in db/db mice. Db/db mice were treated with vehicle (n=20) or ACY775 (10 mg/kg, i.p., once a day, n=22) or ACY738 (50 mg/kg, i.p., once a day, n=20) for 3 weeks. FIG. 5I, Daily body weight (g) of db/db mice treated with Veh, or ACY775 or ACY738 during 3 weeks of treatment period. FIG. 5J, Daily food intake (g) during 3 weeks of treatment. FIG. 5K, Serum leptin (ng/ml) levels after 3 weeks of treatment. FIG. 5L, lean mass (g) FIG. 5M, fat mass (g) and FIG. 5N, fat percentage (%) after 3 weeks of treatment measured by DEXA scan. FIG. 5O, GTT after 1 week of treatment. FIG. 5P, AOC analysis for GTT performed in (FIG. 5O). FIG. 5Q, Blood glucose (mg/dl) levels after 1 week of treatment. AOC analysis for GTT performed in (FIG. 5R). FIG. 5S, Blood glucose (mg/dl) level after 1 week of treatment. FIG. 5T, Serum insulin (ng/ml) levels after 3 weeks of treatment. FIG. 5U, Blood glucose (mg/dl) levels after 3 weeks of treatment. FIG. 5V, Serum insulin (ng/ml) levels after 3 weeks of treatment. FIG. 5W, ITT after 2 weeks of treatment. The results in FIGS. 5I-5Q and 5U-5W were reproduced in two independent experiments. The result in FIG. 5A-5H and 5R-5T were reproduced in two independent experiments.

Values indicate averages±s.e.m. P values were determined by two-way ANOVA with Bonferroni's multiple-comparisons test for curve (FIGS. 5A, 5B, 5G, 5I, 5J, 5O, 5S, 5W) or student' T-test for two groups' comparison (FIGS. 5C-5F, 5H, 5K-5N, 5P-5R, 5T, 5U and 5V). *p<0.05, **p<0.01, ***p<0.001, ****p<0.0001; n.s., not significant (p>0.05).

FIGS. 6A-6X. Tubastatin A, contrary to specific HDAC6 inhibitors ACY775 and ACY738, reduces bodyweight and food intake in DIO HDAC6 knock out mice as well as DIO wild type (WT) mice. DIO WT or HDAC KO mice were treated with specific HDAC6 inhibitors (ACY775 and ACY738) and Tubastatin A for 3 weeks. FIG. 6A, Daily body weight of WT (n=20) or HDAC6-KO (n=20) DIO mice during ACY775 (10 mg/kg/day, i.p.) treatment. FIG. 6B, The first week average food intake (g) of WT or HDAC6-KO mice during ACY775 treatment. FIG. 6C, Blood glucose (mg/dl) levels of ACY775 treated WT or HDAC6-KO mice after 1 week of treatment (n=20 for each group). FIG. 6F, Serum insulin (ng/ml) levels of ACY775 treated WT or HDAC6-KO mice after 3 weeks of treatment (n=8 for each group). FIG. 6G, Serum leptin (ng/ml) levels of WT or HDAC6-KO mice after 3 weeks of ACY775 (n=8) treatment. FIG. 6D, lean mass, FIG. 6E, fat mass, FIG. 6H, fat percentage (%) after 3 weeks of ACY775 treatment measured by DEXA scan.

FIG. 6I, Daily body weight of WT (n=16) or HDAC6-KO (n=20) DIO mice during ACY738 (50 mg/kg/day, i.p) treatment. FIG. 6J, The first week average food intake (g) of WT or HDAC6-KO mice during ACY738 treatment. FIG. 6K, Blood glucose (mg/dl) levels of ACY738 treated WT (n=18) or HDAC6-KO (n=20) mice after one week of treatment. FIG. 6L, Serum insulin (ng/ml) levels of ACY738 treated WT or HDAC6-KO mice after 3 weeks of treatment (n=8 for each group). FIG. 6M, Serum leptin (ng/ml) levels of WT or HDAC6-KO mice after 3 weeks of ACY738 (n=8) treatment. FIG. 6N, Lean mass, FIG. 6O, Fat mass, FIG. 6P, Fat percentage (%) after 3 weeks of ACY738 treatment measured by DEXA scan.

FIG. 6Q, Daily body weight of WT or HDAC6-KO DIO mice during the vehicle or Tubastatin A treatment (25 mg/kg/day, i.p, n=15 for WT-Veh, n=17 for WT-Tubastatin A, and n=18 for HDAC6-KO-Tubastatin A). FIG. 6R, The first week average food intake (g) of WT or HDAC6-KO mice during vehicle or Tubastatin A treatment. FIG. 6S, Blood glucose (mg/dl) levels of Veh- or Tubastatin A-treated WT or HDAC6-KO mice after 1 week of treatment (n=8 for WT-veh, n=9 for WT-Tubastatin A, and n=10 for HDAC6-KO-Tubastatin A). FIG. 6T, Serum insulin (ng/ml) levels of vehicle or Tubastatin A treated WT or HDAC6-KO mice after 2 weeks of treatment (n=12 for WT-veh, n=12 for WT-Tubastatin A, and n=13 for HDAC6-KO-Tubastatin A). FIG. 6U, Serum leptin (ng/ml) levels of WT or HDAC6-KO mice after 3 weeks of vehicle or Tubastatin A treatment (n=12 for WT-Veh, n=13 for WT-Tubastatin A, and n=14 for HDAC6-KO-Tubastatin A). FIG. 6V, Lean mass, FIG. 6W, Fat mass, FIG. 6X, Fat percentage (%) after 3 weeks of Tubastatin A or vehicle treatment.

The results in (FIGS. 6A-6X) were reproduced in two independent experiments. Values indicate averages±s.e.m. P values were determined by two-way ANOVA with Bonferroni's multiple-comparisons test for curve (FIGS. 6A-6C) or student' T-test for two groups' comparison (FIGS. 6D-6X). *p<0.05, **p<0.01, ***p<0.001, ****p<0.0001; n.s., not significant (p>0.05).

FIGS. 7A-7I. Tubastatin A, contrary to specific HDAC6 inhibitors ACY775 and ACY738, improves glucose homeostasis in DIO HDAC6 knock out mice. FIG. 7A, Glucose tolerance test (GTT) after 1 week of ACY775 (10 mg/kg/day, i.p.) treatment in WT (n=17) or HDAC6-KO (n=20) DIO mice. FIG. 7B, Area of curve (AOC) analysis of GTT performed in FIG. 7A). FIG. 7C, Insulin tolerance test (ITT) after 2 weeks of ACY775 (10 mg/kg/day, i.p.) treatment in WT (n=14) or HDAC6-KO (n=18) DIO mice. FIG. 7D, GTT after 1 week of ACY738 (50 mg/kg/day, i.p.) treatment in WT (n=18) or HDAC6-KO (n=22) DIO mice. FIG. 7E, AOC analysis of GTT performed in FIG. 7D. FIG. 7F, ITT after 2 weeks of ACY738 (50 mg/kg/day, i.p.) treatment in WT (n=18) or HDAC6-KO (n=22) DIO mice. FIG. 7G, GTT after 1 week of vehicle or Tubastatin A treatment in WT or HDAC6-KO DIO mice (n=7 for WT-Veh, n=7 for WT-Tubastatin A and n=9 for HDAC6-KO-Tubastatin A). FIG. 7H, AOC analysis of GTT performed in. FIG. 7I, ITT after 2 weeks of vehicle or Tubastatin A treatment in WT or HDAC6-KO DIO mice (n=7 for WT-Veh, n=7 for WT-Tubastatin A and n=9 for HDAC6-KO-Tubastatin A). The results in (FIGS. 7A-7F) were reproduced in two independent cohorts. Values indicate averages±s.e.m. P values were determined by two-way ANOVA with Bonferroni's multiple-comparisons test for curve (FIGS. 7A, FIG. 7C, FIG. 7D, 7F, 7G, 7I) or student' T-test for two groups' comparison (FIGS. 7B, 7E, 7H). *p<0.05, **p<0.01, ***p<0.001, ****p<0.0001; n.s., not significant (p>0.05).

FIGS. 8A-8O are graphs that demonstrate that a poor blood brain barrier permeable HDAC6 inhibitor, Ricolinostat, is not a leptin sensitizer and anti-obesity agent. FIG. 8A is a graph of bodyweight, compared to the control group (FIG. 8A), for Ricolinostat treatment (25 mg/kg, i.p.) alone also did not decrease the bodyweight of DIO mice. FIG. 8B is a graph of the effect on food intake in DIO mice following administration of leptin after pre-treatment with Ricolinostat alone or together with leptin. FIG. 8C is a graph of the effect of leptin injection to Veh- or Ricolinostat-treated DIO mice on STAT3Tyr705. FIGS. 8D-8O are graphs of the results of DIO mice treated with ricolinostat (50 mg/kg, i.p.) for 3 weeks: body weight, food intake (FIGS. 8E, 8F) serum leptin levels (FIG. 8G), lean mass (FIG. 8H), fat mass (FIG. 8I), percent fat (FIG. 8J) blood glucose levels (FIGS. 8K, 8L), glucose tolerance (FIG. 8M) insulin sensitivity (FIG. 8N) and insulin levels (FIG. 8O).

FIGS. 9A and 9B are graphs showing that ACY257 is as effective as ACY775 in lowering body weight (FIG. 9A) and percent change in body weight (FIG. 9B).

FIGS. 10A-10F show the weight lowering effect of bavarostat. Quantification of the ratio of Ac-tubulin to total tubulin signal in the immunoblots is shown in FIG. 10A for liver and FIG. 10B for brain. The results show that bavarostat was equivalent to vehicle in HDAC-6 KO mice.

FIG. 10C is a graph of daily body weight (g) of DIO mice treated with bavarostat (10 mg/kg, i.p.) or vehicle (n=9) or bavarostat (n=9) during 3 weeks of treatment period. FIG. 10D is a graph of daily body weight change (%) of DIO mice during 3 weeks of treatment period; FIG. 10E is a graph of daily food intake (g) during 3 weeks of Veh or bavarostat treatment. FIG. 10F is a graph of weekly averaged 24 h food intake during the Veh or bavarostat treatment. The results show bavarostat was statistically significantly effective in reducing food intake and body weight in DIO mice.

FIGS. 11A-11F show the ineffective weight lowering effect of citarinostat compared to vehicle. Quantification of the ratio of Ac-tubulin to total tubulin signal in the immunoblots is shown in FIG. 11A for liver and FIG. 11B for brain. FIG. 11C is a graph of daily body weight (g) of DIO mice treated with citarinostat (25 mg/kg, i.p.) or vehicle (n=12) or citarinostat (n=11) during 3 weeks of treatment period. FIG. 11D is a graph of daily body weight change (%) of DIO mice during 3 weeks of treatment period; FIG. 11E is a graph of daily food intake (g) during 3 weeks of Veh or citarinostat treatment. FIG. 11F is a graph of weekly averaged 24 h food intake during the Veh or citarinostat treatment.

FIG. 12 is a graph showing that ACY1083 reduced body weight over a 14 day period of time following oral administration.

FIG. 13 is a diagram of signaling pathways of leptin and its downstream effectors. obr oligomerization (here only clinterization shown for reasons of darity) results in phosphorylation and activation of cytoplasmic associated jak2 kinases. These activated jaks phosphorylate tyrosine residues in the cytoplasmic tail of the receptor. Recruitment and activation of secondary signaling molecules allow obr signaling via the jakistat, mapk, pi3k, ampk, and mtor pathways. Diagram is from Wauman, et al. Front. Endocrinol., Sec. Molecular and Structural Endocrinology Vol. 8 (2017) https://doi.org/10.3389/fendo.2017.00030. Inserted into the diagram are where the HDAC6 interacts and sites where the interaction of HDAC6 and Leptin receptor binding can be inhibited or disrupted.

FIGS. 14A-14C are schematics of the sites where the disclosed compounds inhibit or disrupt the HDAC6 or Leptin receptor binding. FIG. 14A is a schematic of binding of Leptin to LepR leads to its tyrosine phosphorylation by Jak2 and subsequently Stat3 phosphorylation and activation. Stat3 plays a crucial role in mediating leptin's appetite suppressing and weight lowering effects. FIG. 14B is a schematic of Hdac6 interaction with LepRb during fasting or obesity reduces LepR, and consequently Stat3 activation. This leads to inhibition of LepR signaling, increased appetite and weight gain. FIG. 14C is a schematic of Hdac6 inhibitors, by blocking association of Hdac6 with LepR, increases LepR activity, suppresses appetite, increases energy expenditure and leads to weight loss.

### DETAILED DESCRIPTION OF THE INVENTION

It has been determined that small molecule (less than 1000 Da) selective inhibitors of HDAC6 (HDAC6 inhibitors resulting in a-tubulin acetylation with no impact on histone acetylation), having a high penetration through the BBB of greater than 0.5, more preferably greater than 1, brain or hypothalamus/plasma ratio, are effective for treatment of obesity, including leptin-resistant obesity, via a central nervous system (CNS) mechanisms of action. In the preferred embodiment, these compounds penetrate into the hypothalamus. Preferred compounds are ACY775 (brain/plasma ratio 1.26), ACY738 (brain/plasma ratio of 1.22), ACY257, ACY1083 and Bavarostat. This contrasts with peripherally acting HDAC6 inhibitors such as ricolinostat (ACY1215) with a brain/plasma ratio of 0.01 and tubastatin A with a brain/plasma ratio of 0.18, and citarinostat which does not inhibit the AgRP nueurons in the hypothalamus. HDAC6 inhibition was tested by treating cells with a high concentration of HDAC6 inhibitor and analyzing for tubulin acetylation, the established biomarker for HDAC6 inhibition.

### I. Definitions

Obesity is defined by the US Center for Disease Control (“CDC”) based on body mass (“BMI”), a person's weight in kilograms divided by the square of height in meters. A BMI between 25.0 to less than 30 is considered overweight. A BMI of 30.0 or higher is within the obesity range. Obesity is frequently subdivided into categories:
Class 1: BMI of 30 to <35
Class 2: BMI of 35 to <40
Class 3: BMI of 40 or higher. Class 3 obesity is categorized as “severe” obesity.

Histones are basic proteins that order and package DNA into nucleosomes (fundamental subunits of chromatin). A nucleosome is an octamer of two of each of four core histones (an H3 (2), H4 (2) tetramer and two H2A and H2B dimers, surrounded by 146 base pairs of DNA). By organizing DNA, histones play a crucial role in the regulation of gene expression. Modification of the four histone tails by reactions such as methylation, acetylation, sumoylation and ubiquitination in the N-terminal of the associated amino acids of H3 and H4 affects transcription, repair and replication. Histones undergo acetylation and deacetylation by the opposing actions of two enzymes, histone acetyl-transferases (HATs) and histone deacetylases (HDACs). The HDAC superfamily consists of 18 members and is organised in four classes of two different protein families:

### Zinc-Dependent Metalloproteins

Class I (HDACs 1-3 and 8)
Class II (HDACs 4-7, 9 and 10, further subdivided into Class IIa (HDACs 4, 5, 7 and 9) and Class IIb (HDACs 6 and 10). Class IIa HDACs continually shuttle between the nucleus and the cytoplasm, whereas Class IIb HDACs are localised in the cytoplasm and contain two catalytic sites.
Class IV (HDAC 11) has similarity to both Class I and Class II.

### NAD+-Dependent Proteins

Class III (SIRT1-7) shows similarity to the yeast Sir2. The class III HDACs are not sensitive to inhibition by HDAC inhibitors and their role in the cell cycle is currently not very clear.

HDACs are classified into four classes based on localization and amino acid sequence similarities, class I HDACs (HDAC1, HDAC2, HDAC3, and HDAC8), class IIa HDACs (HDAC4, HDAC5, HDAC7, and HDAC9), class IIb HDACs (HDAC6 and HDAC10), class II HDACs (HDAC4-7, HDAC9, and HDAC10), class III HDACs (SIRT1-7), and histone deacetylase 11 (HDAC11). While class I HDACs are found in the nucleus, class IIb can travel to the cytoplasm where they can interact with nonhistone proteins.

Cellular HDAC6 inhibition can be verified by treating cells with a high concentration of respective small molecule inhibitors and analyzing for tubulin acetylation, the established biomarker for HDAC6 inhibition. HDAC6 and Sirt2 were identified as the main cytoplasmic tubulin deacetylases (Hubbert, et al. (2002). Nature 417, 455-458. doi: 10.1038/417455a); Inoue, et al. (2007). Oncogene 26, 945-957. doi: 10.1038/sj.onc.1209857.

Selective HDAC6 inhibitors result in α-tubulin acetylation with no impact on histone acetylation.

Selective HDAC6 inhibitors as used herein to reduce or treat leptin resistant obesity are those compounds which can penetrate into the hypothalamus and block interaction of HDAC6 with the leptin receptor, especially at the arctuate neurons in the hypothalamus.

The arcuate nucleus (ARC) is located in the mediobasal hypothalamus and forms a morphological and functional entity with the median eminence (ME), the ARC-ME. The ARC comprises several distinct types of neurons controlling prolactin release, food intake, and metabolism as well as reproduction and onset of puberty. The arcuate nucleus (ARC) of the hypothalamus contains two groups of neurons that express either the neuropeptide proopiomelanocortin (POMC) or coexpress agouti-related protein (AGRP) and neuropeptide Y (NPY). Both groups of neurons express leptin receptors (OBRs) and IRs.

“Analog” or “derivative” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with that specified compound of between 0.5 and 1.0, inclusive, preferably between 0.7 and 1.0, inclusive, most preferably between 0.85 and 1.0, inclusive. A compound is functionally similar to a specified compound, if it induces the same pharmacological effect, physiological effect, or both, as the specified compound. “Analog” or “derivative” can also refer to a modification including, but not limited to, hydrolysis, reduction, or oxidation products, of the disclosed compounds. Hydrolysis, reduction, and oxidation reactions are known in the art. In general, a derivative can be imagined to be formed, at least theoretically, from the parent compound via chemical and/or physical processes. For example, derivatives of HDAC6 inhibitor include compounds possessing one or more substituents affixed to the HDAC6 inhibitor core.

“Metabolite” refers to a compound obtainable from the biochemical processing of another compound. The degradation can be after ingestion or in vitro.

“Pharmaceutically acceptable”, as used herein, refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Prodrug”, as used herein, refers to a pharmacological substance (drug) that is administered to a subject in an inactive (or significantly less active) form. Once administered, the prodrug is metabolized in the body (in vivo) into a compound having the desired pharmacological activity.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Obese,” as used herein, refers to a patient having a body mass index of greater than 30 kg/m². “Overweight” and “Pre-Obese,” as used herein, refer to patients having a body mass index of greater than 25 kg/m². “Morbidly Obese,” as used herein, refers to a patient having a body mass index of greater than 40 kg/m², a body mass index of greater than 35 kg/m² in combination with one or more co-morbidities, a body mass index of greater than 30 kg/m² in combination with uncontrollable diabetes, or combinations thereof.

From the American Cancer Society:

| | BMI | | | | | | | | | | | | | | | | |
|---|---|---|---|---|---|---|---|---|---|---|---|---|---|---|---|---|---|
| | 19 | 20 | 21 | 22 | 23 | 24 | 25 | 26 | 27 | 28 | 29 | 30 | 31 | 32 | 33 | 34 | 35 |
| Height | Weight in Pounds | | | | | | | | | | | | | | | | |
| 4 [symbol] 10 [symbol] | 91 | 96 | 100 | 105 | 11 [symbol] | 11 [symbol] | 119 | 124 | 12 [symbol] | 134 | 13 [symbol] | 1 [symbol] | 14 [symbol] | 153 | 1 [symbol] 8 | 1 [symbol] | 1 [symbol] |
| 4 [symbol] 11 [symbol] | [symbol] | 99 | 10 [symbol] | 10 [symbol] | 114 | 11 [symbol] | 124 | 12 [symbol] | 13 [symbol] | 13 [symbol] | 1 [symbol] 3 | 14 [symbol] | 15 [symbol] | 15 [symbol] | 1 [symbol] | 1 [symbol] | 17 [symbol] |
| 5 [symbol] | [symbol] | 102 | 107 | 112 | 118 | 123 | 12 [symbol] | 12 [symbol] | 13 [symbol] | 142 | 1 [symbol] | 1 [symbol] 3 | 15 [symbol] | 1 [symbol] | 1 [symbol] | 1 [symbol] | 179 |
| 5 [symbol] 1 [symbol] | 100 | 106 | 111 | 11 [symbol] | 12 [symbol] | 127 | 132 | 13 [symbol] | 14 [symbol] | 14 [symbol] | 153 | 15 [symbol] | 164 | 1 [symbol] | 17 [symbol] | 1 [symbol] | 1 [symbol] |
| 5 [symbol] 2 [symbol] | 104 | 10 [symbol] | 115 | 12 [symbol] | [symbol] | 131 | 136 | 14 [symbol] | 14 [symbol] | 1 [symbol] | 15 [symbol] | 1 [symbol] 4 | 1 [symbol] | 175 | 18 [symbol] | 1 [symbol] | 1 [symbol] 1 |
| 5 [symbol] 3 [symbol] | 107 | 1 [symbol] | 11 [symbol] | 124 | 130 | 1 [symbol] | 141 | 1 [symbol] | 15 [symbol] | 1 [symbol] | 1 [symbol] | 1 [symbol] | 175 | 180 | 18 [symbol] | 1 [symbol] | 197 |
| 5 [symbol] 4 [symbol] | 1 [symbol] | 11 [symbol] | 122 | 12 [symbol] | 134 | 140 | 145 | 151 | 15 [symbol] | 1 [symbol] | 1 [symbol] | 174 | 180 | 18 [symbol] | 19 [symbol] | 1 [symbol] | 2 [symbol] |
| 5 [symbol] 5 [symbol] | 1 [symbol] | 120 | 12 [symbol] | 13 [symbol] | 1 [symbol] | 144 | 150 | 156 | 1 [symbol] | 1 [symbol] | 17 [symbol] | 1 [symbol] | 186 | 1 [symbol] | 1 [symbol] | 20 [symbol] | 210 |
| 5 [symbol] 6 [symbol] | 1 [symbol] | 1 [symbol] | 130 | 13 [symbol] | 1 [symbol] | 14 [symbol] | 155 | 1 [symbol] | 16 [symbol] | 173 | 17 [symbol] | 1 [symbol] | 192 | 1 [symbol] | 20 [symbol] | 210 | 216 |
| 5 [symbol] 7 [symbol] | 121 | 127 | 134 | 140 | 14 [symbol] | 15 [symbol] | 15 [symbol] | 16 [symbol] | 1 [symbol] | 178 | 185 | 1 [symbol] 1 | 198 | 204 | 211 | 217 | 2 [symbol] |
| 5 [symbol] 8 [symbol] | 125 | 1 [symbol] 1 | 13 [symbol] | 144 | 15 [symbol] | 15 [symbol] | 16 [symbol] | 171 | 1 [symbol] | 1 [symbol] 4 | 190 | 1 [symbol] | 2 [symbol] 3 | 210 | 21 [symbol] | 22 [symbol] | 230 |
| 5 [symbol] 9 [symbol] | 12 [symbol] | 1 [symbol] | 142 | 14 [symbol] | 1 [symbol] | 162 | 1 [symbol] | 17 [symbol] | 1 [symbol] | 1 [symbol] 9 | 196 | 2 [symbol] | 209 | 216 | 22 [symbol] | 2 [symbol] | 2 [symbol] |
| 5 [symbol] 10 [symbol] | 1 [symbol] | 1 [symbol] | 1 [symbol] | 15 [symbol] | 1 [symbol] | 167 | 174 | 18 [symbol] | 1 [symbol] | 1 [symbol] | 2 [symbol] 2 | 2 [symbol] | 21 [symbol] | 222 | 22 [symbol] | 2 [symbol] | 2 [symbol] |
| 5 [symbol] 11 [symbol] | 13 [symbol] | 143 | 150 | 15 [symbol] | 1 [symbol] | 172 | 179 | 1 [symbol] | 1 [symbol] | 200 | 20 [symbol] | 21 [symbol] | 222 | 229 | 2 [symbol] | 2 [symbol] | 250 |
| 6 [symbol] | 140 | 1 [symbol] | 154 | 16 [symbol] | 1 [symbol] | 177 | 18 [symbol] | 191 | 1 [symbol] | 20 [symbol] | 213 | 221 | 22 [symbol] | 2 [symbol] | 242 | 2 [symbol] | 2 [symbol] |
| 6 [symbol] 1 [symbol] | 144 | 151 | 15 [symbol] | 16 [symbol] | 174 | 182 | 18 [symbol] | 197 | 2 [symbol] | 212 | 219 | 227 | 235 | 242 | 25 [symbol] | 2 [symbol] | 2 [symbol] |
| 6 [symbol] 2 [symbol] | 14 [symbol] | 1 [symbol] | 1 [symbol] | 171 | 17 [symbol] | 1 [symbol] | 1 [symbol] | 2 [symbol] 2 | 2 [symbol] | 218 | 22 [symbol] | 2 [symbol] | 241 | 2 [symbol] | 2 [symbol] | 2 [symbol] | 2 [symbol] |
| 6 [symbol] 3 [symbol] | 1 [symbol] | 1 [symbol] | 1 [symbol] | 176 | 1 [symbol] 4 | 192 | 20 [symbol] | 2 [symbol] | 2 [symbol] | 224 | 2 [symbol] 2 | [symbol] | 2 [symbol] | 25 [symbol] | 2 [symbol] | 272 | 2 [symbol] |

| | | |
|---|---|---|
| Healthy Weight | Overweight | Obese |

Source: US Department of Health and Human Services, National Institutes of Health, National Health, L [symbol] , and Blood [symbol] The Clinical Guidelines on the Identification, Eval [symbol] and Treatment of Overweight and Obesity in A [symbol] Evidence Repo [symbol] September 19 [symbol] [N [symbol] p [symbol] N [symbol] 9 [symbol] -4 [symbol] ]. [symbol]
indicates data missing or illegible when filed

“Effective amount” or “therapeutically effective amount”, as used herein, refers to an amount of a selective HDAC6 inhibitor that is effective to induce weight loss in a pre-obese, obese, or morbidly obese patient, reduce body fat in a pre-obese, obese, or morbidly obese patient, reduce food intake in a pre-obese, obese, or morbidly obese patient, improve glucose homeostasis in a pre-obese, obese, or morbidly obese patient, prevent weight gain and/or prevent an increase in body mass index in a normal, pre-obese, obese, or morbidly obese patient, or combinations thereof.

### II. Pharmaceutical Formulations

Lysine deacetylase 6 (HDAC6) is a class IIB Zn2+ deacetylase. Among the metal-dependent HDAC isozymes, HDAC6 is unique in that it contains two catalytic domains, CD1 and CD2. The first domain specifically deacetylates acetylated C-terminal lysine residues, while the second shows a particularly broad substrate selectivity. CD2 is a tubulin deacetylase and a tau deacetylase. The development of HDAC6-selective inhibitors has focused exclusively on this domain. In contrast, there is a dearth of structural and functional information regarding CD1, which exhibits much narrower substrate specificity in comparison with CD2. The active site of HDAC6 CD1 is wider than that of CD2, which is unexpected in view of the narrow substrate specificity of CD1. Amino acid substitutions between HDAC6 CD1 and CD2, as well as conformational differences in conserved residues, define striking differences in active site contours. Catalytic activity measurements with HDAC6 CD1 confirm the preference for peptide substrates containing C-terminal acetyl lysine residues. However, these measurements also show that CD1 exhibits weak activity for peptide substrates bearing certain small amino acids on the carboxyl side of the scissile acetyl-lysine residue. Taken together, these results establish a foundation for understanding the structural basis of HDAC6 CD1 catalysis and inhibition, for the development of HDAC6 CD1-selective inhibitors.

There is evidence that HDAC6 catalyzes deacetylation of several proteins involved in a variety of cellular processes. Among them, HDAC6-mediated deacetylation of α-tubulin regulates microtubule stability and cell motility. Another characterized substrate, cortactin, binds to deacetylated actin filaments and participates in the fusion of lysosomes and autophagosomes. The enzyme also plays a role in protein folding by regulating the activity of the Hsp90 chaperone protein via deacetylation. In addition, HDAC6 is an important player in innate immunity, regulating the detection of pathogen genomic material via deacetylation of retinoic acid inducible gene-I protein. Studies have now determined that HDAC6 inhibitors that pass through the BBB into the hypothalamus are most efficacious for treatment of obesity and alleviation of leptin insensitivity.

The BBB is a highly dynamic and complex structure formed by the specialized endothelial cells of the brain capillaries and the ependymal cells of the circumventricular organs. These cells establish an interphase between the blood in the cerebral vessels on one side and the intercellular fluid of the brain parenchyma and the cerebrospinal fluid (CSF), on the other. Tight junctions between cells forming this interphase represents a fundamental physical barrier preventing free movements of compounds through the intercellular spaces between endothelial cells in the BBB and ependymal cells in the choroid plexuses blood-CSF barrier. Neurons secreting hormones into the blood stream must be in open communication with the blood capillaries. In the central nervous system (CNS), there are discrete areas localized in the ventricular walls, known as circumventricular organs (CVO).

These “brain windows” may serve two purposes, namely, to allow peptides and proteins secreted by the neural tissue to reach the blood stream, and to allow neural cells to sense the plasma. The CVO share some characteristics, namely, their location close or within the ventricles; their vascular supply formed by capillaries endowed with fenestrations and a perivascular space, and their ependymal cells highly specialized in transport and/or secretion. With the exception of the choroid plexuses of the lateral ventricles, all other CVOs display the unique feature of being single, unpaired structures located along the midline of the CNS CVOs, they may be grouped into the following: (i) sensing organs (vascular organ of the lamina terminalis, subfornical organ, area postrema): The cell body and dendrites of the neurons forming these CVOs are not protected by a BBB and remain in open communication with peripheral blood so that they may respond to blood borne signals, such as the subfornical neurons responding to angiotensin II plasma levels. The axons of the neurons of these sensory CVOs project to multiple areas of the CNS; therefore, such axons leave the BBB-free area of the CVO and enter the brain areas protected by the BBB. This is different from providing direct access of the substances into the CNS. (ii) Neurosecretory organs (pineal gland, median eminence and neural lobe of the pituitary): In the medial hypothalamus the cell body and dendrites of the neurons secreting neuropeptides and monoamines are protected by the BBB whereas their axons enter the BBB-free areas of the median eminence and neural lobe to deliver their secretions into the portal capillaries and systemic capillaries, respectively. (iii) Ependymal secretory organs (sub-commissural organ, choroid plexuses): These CVOs are formed by ependymal cells highly specialized to secrete proteins into the CSF. (iv) Transporting CVOs organs (choroid plexuses, median eminence). The choroidal cells and the tanycytes of the median eminence are cells endowed with a transport machinery to transfer substances from blood to CSF and from CSF to blood.

It has been discovered that HDAC6 inhibitors with high levels of penetration of the BBB, especially the hypothalamus, especially inhibitors which block LepRb-HDAC6 interaction, are effective in treating leptin-resistant obesity, as well as to increase leptin sensitivity. It has been further discovered that the critical region of the brain for the inhibitors to be effective is the hypothalamus. Several compounds were tested and it was determined that a central, not peripheral, mechanism of action was involved with the HDAC6 inhibitors that had high levels of penetration through the blood brain barrier and/or into the hypothalamus. Analysis of hypothalamic gene expressions in fed, starved and obese mice identified correlations with gene expression changes induced by small molecule inhibition of HDAC6 activity, indicating that pharmacological inhibition of HDAC6 activity in the hypothalamus is effective for the treatment of obesity and related disorders. Subsequent studies in high fat diet induced mice treated with HDAC6 isoform-specific inhibitors, ACY738 and ACY775, showed these compounds caused significant reduction of food intake and total body fat mass. Effects were greater for the more selective HDAC6 inhibitor ACY775 as compared to ACY738. Significant weight loss was also demonstrated by ACY1083, bavarostat and a compound that inhibits HDAC6 in the hypothalamus but has low BBB penetration, ACY257.

Only HDAC6 inhibitors which penetrate the hypothalamus and inhibit HDAC6 therein are effective to treat obesity, especially leptin-resistant obesity. Data demonstrates that a compound, ACY 257, which inhibits HDAC6 in the hypothalamus but does not inhibit HDAC6 in other brain regions such as cerebrum, frontal lobe, temporal lobe, and brainstem, greatly reduces the body weight. The hypothalmus is a very small part of the brain. The hypothalamus has a slightly more permeable blood brain barrier compared to other parts of the brain. The data indicates that inhibitors of HDAC6 which have the ability to preferentially pass through the BBB and into the hypothalamic region are able to reduce body weight.

Based on these studies, it was determined that small molecule (less than 1000 Da) selective inhibitors of HDAC6 (inhibitors result in α-tubulin acetylation with no impact on histone acetylation), especially inhibitors which block LepRb-HDAC6 interaction, having a high penetration through the BBB of greater than 1 brain/plasma ratio or of the hypothalamus, are most effective for treatment of obesity, including leptin-resistant obesity, via a central nervous system (CNS) mechanism of action. Preferred HDAC6 inhibitors have >0.3, >0.5, or >1 brain or hypothalamus/plasma concentration. More preferably, the HDAC6 inhibitors have >0.3, >0.5, or >1 hypothalamus/plasma concentration. Most preferably, HDAC6 inhibitors which have >0.3, >0.5, >1 Arcuate Nucleus/plasma concentration. The arcuate nucleus has the highest BBB permeability in the hypothalamus and is around 1/10,000 of the total area of the brain. The results indicate that inhibiting HDAC6 in the AgRP neurons in the arcuate nucleus is sufficient to create the weight loss. If an HDAC6 inhibitor just gets to the arcuate nucleus at concentrations that can inhibit HDAC6 activity, but not to the other regions of the brain, total brain/plasma or hypothalamus/plasma concentrations may be low. However, the arcuate nucleus concentration will be higher than total brain/plasma and hypothalamus/plasma concentrations.

Preferred compounds are ACY775 (brain/plasma ratio 1.26) and ACY738 (brain/plasma ratio of 1.22). This contrasts with peripherally acting HDAC6 inhibitors such as ricolinostat (ACY1215) with a brain/plasma ratio of 0.01 and tubastatin A with a brain/plasma ratio of 0.18, neither of which have high penetrance into the hypothalamus nor show HDAC6 inhibition in the hypothalamus. HDAC6 inhibition was tested by treating cells with a high concentration of HDAC6 inhibitor and analyzing for tubulin acetylation, the established biomarker for HDAC6 inhibition.

### A. HDAC6 Inhibitors and Formulations

HDAC6 inhibitors are commercially available from suppliers such as Sigma Aldrich, Sellect Chemistry, AdooQ Bioscience, and others.

Preferred HDAC6 inhibitors interfere with leptin signaling and LepRb deacetylation.

Small molecule (less than 1000 Da) selective inhibitors of HDAC6, having a high penetration through the BBB of greater than 0.5 (brain/plasma ratio), more preferably greater than 1, brain/plasma ratio, are most effective for treatment of obesity, including leptin-resistant obesity, via a central nervous system (CNS) mechanisms of action. Preferred compounds are ACY775 (brain/plasma ratio 1.26) and ACY738 (brain/plasma ratio of 1.22).

This contrasts with compounds that are not useful including peripherally acting HDAC6 inhibitors such as ricolinostat (ACY1215) with a brain/plasma ratio of 0.01 and tubastatin A with a brain/plasma ratio of 0.18, nor citarinostat (ACY241) or ricolinostat (ACY1215).

HDAC6 inhibition was tested by treating cells with a high concentration of selective HDAC6 inhibitor and analyzing for tubulin acetylation, the established biomarker for HDAC6 inhibition.

Compounds which are not useful due to low brain/plasma ratios, or inability to inhibit HDAC6 in the hypothalmus:

Ricolinostat (ACY1215) Brain/plasma ratio<0.01 https://actaneurocomms.biomedcentral.com/articles/10.1186/s40478-018-0604-3

Tubastatin A brain/plasma ratio ˜0.18 https://www.nature.com/articles/npp2013207

Brain:plasma ratios of useful compounds including:

ACY738 inhibits HDAC6 with low nanomolar potency (IC50=1.7 nM) and has a selectivity of 60- to 1500-fold over class I HDACs. The brain/plasma ratio is ˜1.22 https://www.nature.com/articles/npp2013207

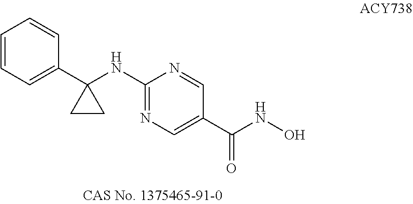 [SMILES: O=C(NO)c1cnc(NC2(c3ccccc3)CC2)nc1]

ACY775 brain/plasma ratio ˜1.26 https://www.nature.com/articles/npp2013207

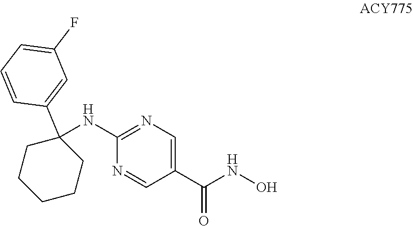 [SMILES: O=C(NO)c1cnc(NC2(c3cccc(F)c3)CCCCC2)nc1]

| Synonyms | N/A |
|---|---|
| Formula | C₁₇H₁₉FN₄O₂ |
| Molecular Weight | 330.36 |

ACY-1083 is a selective and brain--penetrating HDAC6 inhibitor with an IC50 of 3 nM and is 260-fold more selective for HDAC6 than all other classes of HDAC isoforms.

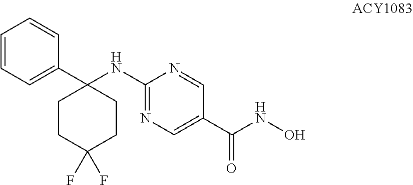 [SMILES: O=C(NO)c1cnc(NC2(c3ccccc3)CCC(F)(F)CC2)nc1]

Bavarostat (4-(((((3r,5r,7r)-adamantan-1-yl)methyl)(methyl)amino)methyl)-3-fluoro-N-hydroxybenzamide) is an HDAC6-selective inhibitor with high brain penetrance, Strebl, et al. ACS Cent Sci. 2017 Sep. 27; 3(9): 1006-1014.

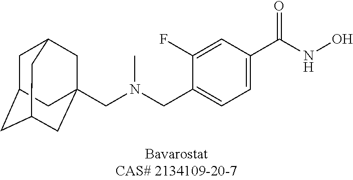 [SMILES: CN(Cc1ccc(C(=O)NO)cc1F)CC12CC3CC(CC(C3)C1)C2]

HDAC6 inhibitor, ACY-257, shows limited brain expression when administered systemically (Selleck Chemical).

| | HDAC1 | HDAC2 | HDAC3 | HDAC6 | | | Plasma | AUC Brain/ |
|---|---|---|---|---|---|---|---|---|
| | IC₅₀ | IC₅₀ | IC₅₀ | IC₅₀ | Cmax | T1/2 | AUCₗₐₛₜ | AUC Plasma |
| ACY-257 Structure | nM | nM | nM | nM | ng/ml | (h) | (hr*ng/ml) | Ratio |
| 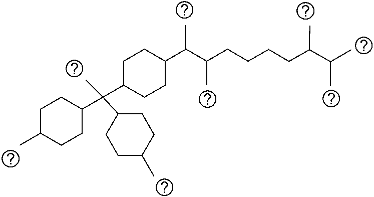 [SMILES: CC1CCC(C(C)(C2CCC(C)CC2)C2CCC(C(C)C(C)CCCCC(C)C(C)C)CC2)CC1] | 268 | 530 | 850 | 12 | 1047 | 8.15 | 871 (5 mg/kg i.p.) | 0.01 |

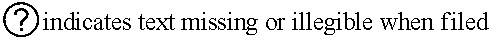

### ACY257

### B. Additional Therapeutics

In some cases, the pharmaceutical formulation can further contain one or more additional active agents. Examples include anti-obesity drugs that act by HDAC6 independent mechanisms of action, for example GLP1R agonists, GIP Receptor Agonist, GLP1/GIP Receptor Dual agonists, etc. Compounds for treatment of diabetes and for control of glucogenesis may also be important.

The FDA has approved five drugs for long term use: orlistat, phentermine-topiramate, naltrexone-bupropion, liraglutide, and sernagiutide.

In certain embodiments, the pharmaceutical formulations further contain leptin, a leptin analog, or combinations thereof.

Leptin is a peptide hormone that serves as the afferent signal in a negative feedback loop regulating food intake and body weight in vivo. Unprocessed human leptin is synthesized in vivo as a 167 amino acid, 16 kDa protein prohormone. Unprocessed leptin includes an N-terminal 21-amino acid signal sequence that is cleaved from the remainder of the polypeptide to generate mature, circulating, leptin (containing 146 amino acids).

The terms “leptin” and “leptin analog,” as used herein, encompass naturally occurring human leptin, naturally occurring leptin produced by a non-human species such as a mouse or rat, recombinantly produced mature leptin, such as metreleptin (i.e., recominbinant methionyl human leptin or r-metHuLeptin, which is a 147 amino acid leptin analog generated by the genetically engineered N-terminal addition of a methionine to the N-terminal amino acid of the 146-amino acid, mature, circulating, human leptin), as well as leptin fragments, leptin variants, leptin fusion proteins, and other derivatives thereof known in the art to possess biological activity. Exemplary leptin variants include those where the amino acid at position 43 is substituted with Asp or Glu; position 48 is substituted Ala; position 49 is substituted with Glu, or absent; position 75 is substituted with Ala; position 89 is substituted with Leu; position 93 is substituted with Asp or Glu; position 98 is substituted with Ala; position 117 is substituted with Ser, position 139 is substituted with Leu, position 167 is substituted with Ser, and any combination thereof.

In certain embodiments, the pharmaceutical formulation includes r-metHuLeptin (A-100, METRELEPTIN®), available from Amylin Pharmaceuticals (San Diego, Calif.).

Pharmaceutical formulations can also include one or more vitamins, minerals, dietary supplements, nutraceutical agents, such as proteins, carbohydrates, amino acids, fatty acids, antioxidants, and plant or animal extracts, or combinations thereof. Suitable vitamins, minerals, nutraceutical agents, and dietary supplements are known in the art, and disclosed, for example, in Roberts et al., (Nutriceuticals: The Complete Encyclopedia of Supplements, Herbs, Vitamins, and Healing Foods, American Nutriceutical Association, 2001). Nutraceutical agents and dietary supplements are also disclosed in Physicians' Desk Reference for Nutritional Supplements, 1st Ed. (2001) and The Physicians' Desk Reference for Herbal Medicines, 1st Ed. (2001).

### C. Formulations

The selective HDAC6 inhibitors are preferably administered to a mucosal surface, most preferably orally, buccally, or nasally. These can be formulated using known excipients. Formulations may also be formulated for sustained, delayed, and/or pulsatile release to deliver an effective amount of HDAC6 inhibitor to cause weight loss. They are preferably administered once or twice daily. Dosage is based on weight. Typical dosages will be in the range of 25 to 500 mg/day.

The pharmaceutical formulations can be administered to induce weight loss in a pre-obese, obese, or morbidly obese patient, reduce body fat in a pre-obese, obese, or morbidly obese patient, reduce food intake in a pre-obese, obese, or morbidly obese patient, improve glucose homeostasis in a pre-obese, obese, or morbidly obese patient, or combinations thereof. In some cases, a pharmaceutical formulation containing one or more of the HDAC6 inhibitors is administered to a pre-obese, obese, or morbidly obese patient in a therapeutically effective amount to induce weight loss, preferably in a therapeutically effective amount and time of administration to decrease body mass or body fat by at least 10%, more preferably by at least 15%, most preferably by at least 20%, or higher. In some cases, a pharmaceutical formulation containing one or more of the HDAC6 inhibitors is administered to a pre-obese, obese, or morbidly obese patient in a therapeutically effective amount to reduce food intake, appetite, or combinations thereof, preferably in a therapeutically effective amount to reduce average daily food intake (in terms of calories). In some cases, a pharmaceutical formulation containing one or more of the selective HDAC6 inhibitors is administered to a pre-obese, obese, or morbidly obese patient in a therapeutically effective amount to improve glucose homeostasis, preferably in a therapeutically effective amount to reduce average fasting plasma blood glucose. In cases where the pharmaceutical formulations are administered to normalize blood sugar, the formulations are preferably administered in an amount effective to lower blood glucose levels to less than about 180 mg/dL. The formulations can be co-administered with other anti-diabetic therapies, if necessary, to improve glucose homeostasis.

Pharmaceutical formulations contain a therapeutically effective amount of a selective HDAC6 inhibitor having a high degree of penetration through the blood brain barrier (“BBB”), resulting in a brain or hypothalamus/plasma ratio of greater than 0.5, more preferably greater than 1, in combination with one or more pharmaceutically acceptable excipients, wherein the selective HDAC6 inhibitor is present in an effective amount to cause weight loss in an individual having a BMI of 25 or more. It will be understood that the dosage may be formulated into a single unit (capsule, tablet, microparticles that are encapsulated or suspended in a liquid, pessary) or be in a form that requires administration of more than one unit to provide an effective dosage, or be in a form such as a liquid or gel for oral administration or injection, where the liquid is measured to determine the effective amount.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials that are generally recognized as safe (GRAS), and may be administered to an individual without causing undesirable biological side effects or unwanted interactions.

In the preferred embodiment, the formulations are for enteral administration—orally, or to another mucosal surface such as the nasal cavities, lung, sublingual or buccal, vaginal, rectal, or pulmonary.

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations may be prepared using one or more pharmaceutically acceptable excipients, including diluents, preservatives, binders, lubricants, disintegrators, swelling agents, fillers, stabilizers, and combinations thereof. Excipients, including plasticizers, pigments, colorants, stabilizing agents, and glidants, may also be used to form coated compositions for enteral administration. Delayed release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Diluents, also referred to as “fillers,” are typically necessary to increase the bulk of a solid dosage form so that a practical size is provided for compression of tablets or formation of beads and granules. Binders are used to impart cohesive qualities to a solid dosage formulation, and thus ensure that a tablet or bead or granule remains intact after the formation of the dosage forms. Lubricants are used to facilitate tablet manufacture. Disintegrants are used to facilitate dosage form disintegration or “breakup” after administration. Stabilizers are used to inhibit or retard drug decomposition reactions that include, by way of example, oxidative reactions.

The selective HDAC6 inhibitor can be formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a solid oral dosage form, such as a tablet or capsule, and the solid dosage form is coated with one or more controlled release coatings, such as a delayed release coatings or extended release coatings. The coating or coatings may also contain the compounds and/or additional active agents.

The formulation may provide for extended release. The extended release formulations are generally prepared as diffusion or osmotic systems, for example, as described in “Remington — The science and practice of pharmacy” (20th ed., Lippincott Williams & Wilkins, Baltimore, MD, 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and CARBOPOL® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof. The plastic material can be a pharmaceutically acceptable acrylic polymer, such as acrylic acid or methacrylic acid copolymers known in the art. One common example is commercially available from Rohm Pharma as an EUDRAGIT®

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Delayed release formulations can be created by coating a solid dosage form with a polymer film, which is insoluble in the acidic environment of the stomach, and soluble in the neutral environment of the small intestine. The delayed release dosage units can be prepared, for example, by coating a drug or a drug-containing composition with a selected coating material. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

The formulation can provide pulsatile delivery of the one or more of the compounds disclosed herein. By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

The compounds can be formulated for parenteral administration. “Parenteral administration”, as used herein, means administration by any method other than through the digestive tract or non-invasive topical or regional routes. For example, parenteral administration may include administration to a patient intravenously, intradermally, intramuscularly, or subcutaneously.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

For parenteral administration, the compounds, and optionally one or more additional active agents, can be incorporated into microparticles, nanoparticles, or combinations thereof that provide controlled release. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

In depot formulations containing a polymeric or oligomeric carrier, the carrier and active agent can be formulated as a solution, an emulsion, or suspension. One or more selective HDAC6 inhibitors, and optionally one or more additional active agents, can also be incorporated into polymeric or oligomeric microparticles, nanoparticles, or combinations thereof.

In some cases, the formulation is fluid and designed to solidify or gel (i.e., forming a hydrogel or organogel) upon injection. This can result from a change in solubility of the composition upon injection, or for example, by injecting a pre-polymer mixed with an initiator and/or crosslinking agent. The polymer matrix, polymer solution, or polymeric particles entrap the active agent at the injection site. As the polymeric carrier is gradually degraded, the active agent is released, either by diffusion of the agent out of the matrix and/or dissipation of the matrix as it is absorbed. The release rate of the active agent from the injection site can be controlled by varying, for example, the chemical composition, molecular weight, crosslink density, and/or concentration of the polymeric carrier. Examples of such systems include those described in U.S. Pat. Nos. 4,938,763, 5,480,656 and 6,113,943.

Depot formulations can also be prepared by using other rate-controlling excipients, including hydrophobic materials, including acceptable oils (e.g., peanut oil, corn oil, sesame oil, cottonseed oil, etc.) and phospholipids, ion-exchange resins, and sparingly soluble carriers.

The liquid formulations described above can also be administered using a nebulizer. Nebulizers are liquid aerosol generators that convert the liquid formulation described able, usually aqueous-based compositions, into mists or clouds of small droplets, preferably having diameters less than 5 microns mass median aerodynamic diameter, which can be inhaled into the lower respiratory tract. This process is called atomization. The droplets carry the one or more active agents into the nose, upper airways or deep lungs when the aerosol cloud is inhaled. Any type of nebulizer may be used to administer the formulation to a patient, including, but not limited to pneumatic (jet) nebulizers and electromechanical nebulizers.

### III. Methods of Treatment

Pharmaceutical formulations containing one or more of the selective HDAC6 inhibitors can be administered to induce weight loss in a pre-obese, obese, or morbidly obese patient, reduce body fat in a pre-obese, obese, or morbidly obese patient, reduce food intake in a pre-obese, obese, or morbidly obese patient, improve glucose homeostasis in a pre-obese, obese, or morbidly obese patient, prevent weight gain and/or prevent an increase in body mass index in a normal, pre-obese, obese, or morbidly obese patient, or combinations thereof.

In certain embodiments, the pharmaceutical formulations are administered to a patient suffering from obesity (e.g., a pro-obese, obese, or morbidly obese patient), an obesity-related disease or disorder, diabetes, insulin-resistance syndrome, lipodystrophy, nonalcoholic steatohepatitis, a cardiovascular disease, polycystic ovary syndrome, or a metabolic syndrome.

In cases where the pharmaceutical formulations are administered to normalize blood sugar, the formulations are preferably administered in an amount effective to lower blood glucose levels to less than about 180 mg/dL.

The formulations can be co-administered with other anti-diabetic therapies, if necessary, to improve glucose homeostasis.

Pharmaceutical formulations may also be administered to patients suffering from a disease or disorder that causes obesity or predisposes a patient to become obese, such as Prader Willi Syndrome (PWS), Bardet-Biedl syndrome or a mutation in the gene encoding for the melanocortin receptor 4 (MC4R) protein (i.e., an MC4R mutation).

### A. Dosages

The precise dosage administered to a patient will depend on many factors, including the physical characteristics of the patient (e.g., weight), the degree of severity of the disease or disorder to be treated, and the presence or absence of other complicating diseases or disorders and can be readily determined by the prescribing physician.

In certain embodiments, the selective HDAC6 inhibitor is administered at a dosage equivalent to an oral dosage of between about 0.005 mg and about 500 mg per kg of body weight per day, more preferably between about 0.05 mg and about 100 mg per kg of body weight per day, most preferably between about 0.1 mg and about 10 mg per kg of body weight per day. In particular, embodiments, the selective HDAC6 inhibitor is administered at a dosage equivalent to an oral dosage of between about 1.0 mg and 5.0 mg per kg of body weight per day. Typical dosages will be in the range of 25 to 500 mg/day, depending on the weight of the person taking the formulation. Dosages may be modified depending on factors such as route of administration, co-administration with other agents, leptin-resistance, presence of other disorders, and rate of weight loss desired.

In some cases, a pharmaceutical formulation containing one or more of the selective HDAC6 inhibitors is administered to a pre-obese, obese, or morbidly obese patient in a therapeutically effective amount to induce weight loss. In certain embodiments, a pharmaceutical formulation containing one or more of the selective HDAC6 inhibitors is administered to a pre-obese, obese, or morbidly obese patient in a therapeutically effective amount to decrease body mass by at least 10%, more preferably by at least 15%, most preferably by at least 20%.

In some cases, a pharmaceutical formulation containing one or more of the selective HDAC6 inhibitors is administered to a pre-obese, obese, or morbidly obese patient in a therapeutically effective amount to reduce body fat. In certain embodiments, a pharmaceutical formulation containing one or more of the selective HDAC6 inhibitors is administered to a pre-obese, obese, or morbidly obese patient in a therapeutically effective amount to decrease body fat by at least 10%, more preferably by at least 15%, most preferably by at least 20%.

In some cases, a pharmaceutical formulation containing one or more of the selective HDAC6 inhibitors is administered to a pre-obese, obese, or morbidly obese patient in a therapeutically effective amount to reduce food intake, appetite, or combinations thereof. In certain embodiments, a pharmaceutical formulation containing one or more of the selective HDAC6 inhibitors is administered to a pre-obese, obese, or morbidly obese patient in a therapeutically effective amount to reduce average daily food intake (in terms of calories) by at least 15%, 17%, 20%, 22%, 25%, 28%, 30%, 32%, 35%, or greater.

In some cases, a pharmaceutical formulation containing one or more of the selective HDAC6 inhibitors is administered to a pre-obese, obese, or morbidly obese patient in a therapeutically effective amount to improve glucose homeostasis. In certain embodiments, a pharmaceutical formulation containing one or more of the selective HDAC6 inhibitors is administered to a pre-obese, obese, or morbidly obese patient in a therapeutically effective amount to reduce average fasting plasma blood glucose by at least 10%, 12%, 15%, 18%, 20%, 22%, 25%, or greater. In cases where the pharmaceutical formulations are administered to normalize blood sugar, the formulations are preferably administered in an amount effective to lower fasting plasma glucose levels to less than about 180 mg/dL, more preferably less than about 160 mg/dL, more preferably less than about 140 mg/dL.

### B. Therapeutic Administration

Pharmaceutical formulations may be administered, for example, in a single dosage, as a continuous dosage, one or more times daily, or less frequently, such as once a week. The pharmaceutical formulations can be administered once a day or more than once a day, such as twice a day, three times a day, four times a day or more. In certain embodiments, the formulations are administered orally, once daily or less.

The pharmaceutical formulations are administered in an effective amount and for an effective period of time to elicit the desired therapeutic benefit. In certain embodiments, the pharmaceutical formulation is administered daily, bi-weekly, weekly, bi-monthly or monthly for a period of at least one week, two weeks, three weeks, four weeks, one month, two months, three months, four months, five months, six months, seven months, eight months, nine months, ten months, eleven months, one year, or longer.

The HDAC6 inhibitors can be co-administered with one or more additional therapeutic, prophylactic, or diagnostic agents. Co-administration includes administration within the same dosage form or within different dosage forms. For those embodiments where the compounds described herein and the one or more additional therapeutic, prophylactic, or diagnostic agents are administered in different dosage forms, the dosage forms can be administered simultaneously (e.g., at the same time or essentially at the same time) or sequentially. “Essentially at the same time” as used herein generally means within ten minutes, preferably within five minutes, more preferably within two minutes, most preferably within in one minute. Dosage forms administered sequentially can be administered within several hours of each other, e.g., with ten hours, nine hours, eight hours, seven hours, six hours, five hours, four hours, three hours, two hours, one hour, 30 minutes, 20 minutes, or 15 minutes.

In certain embodiments, the selective HDAC6 inhibitors described herein are co-administered with leptin or a leptin analog. In these cases, leptin or a leptin analog may be co-administered with the selective HDAC6 inhibitors for a portion of the treatment period, or during the entirety of the treatment period. In preferred embodiments, the selective HDAC6 inhibitors are co-administered with r-metHuLeptin (A-100, METRELEPTIN®), available from Amylin Pharmaceuticals (San Diego, Calif.).

In certain embodiments, the patients have diabetes. In these cases, the selective HDAC6 inhibitors described herein may be co-administered with one or more therapies for diabetes.

The present invention will be further understood by reference to the following non-limiting examples.

### Example 1: Selection Process for HDAC6 Inhibitors as Possible Leptin Sensitizers

A process for selecting Celastrol-regulated hypothalamic genes involved in creation of satiety was tested. Expression of genes that did not change between Vehicle (vehicle) and Celastrol (CS)-treated groups, but which were regulated in the Pair-fed (PF) group versus CS-treated groups, were predicted to be the genes that regulate orexigenic or anorexigenic stimuli.

### Materials and Methods

Four groups of diet induced obese (DIO) mice were examined: 1) treated either with vehicle with ad libitum to food, or 2) pair-fed to CS-treated group, 3) treated with CS with ad libitum to food, or 4) treated with CS and given the same amount of food and at same intervals as the PF group (CS/PF).

RNA sequencing was performed from the hypothalamus of the mice after 4 days of treatment and analysis conducted for principal component analysis (PCA) for RNA sequencing data. and cluster analysis of overlapping genes which had significant difference among the comparison of PF to Vehicle, PF to CS and PF to CS/PF. The cutoff of false discovery rate (FDR) of <0.3 was used for combined analysis. The celastrol regulated gene signature was compared to PF and Vehicle. The analysis of gene signatures identified was put into a Connectivity Map (CMAP) L1000 platform. Signature of celastrol-regulated genes was used as the query.

### Results

The HDAC6 inhibitor signature in L1000 platform has the highest enriching score compared to Celastrol regulated hypothalamic gene signature. Immunostaining for endogenous HDAC6 protein in arcuate nucleus (Arc) of WT lean (12 weeks of age) and DIO mice (18 weeks of HFD feeding) at fed or after overnight (15h) fasting conditions was quantified by average HDAC6 fluorescence intensity from lean (n=4) and DIO (n=5). Immunostaining for HDAC6 and GFP in Agrp-ires-cre::L-S-L-cas9-GFP mice at fed and fasted conditions was conducted using GFP as a reporter for AgRP-expressing neurons in the arcuate nucleus (Arc). The ratio of HDAC6/GFP co-positive cells' number to number of GFP positive cells. (n=3 for fed mice, n=4 for fasted mice) was determined.

Arcuate nucleus harbors two potentially important sub-population of neurons: the AgRP- and POMC-expressing neurons. Of these neurons, AgRP-expressing neurons have been shown to mediate the majority of leptin's anorexigenic effects. HDAC6 protein levels analyzed in fed and fasted conditions in mice, which expressed GFP under copy*oter, showed a strong correlation, indicating that fasting induced HDAC6 mainly happens in the AgRP-expressing neurons of arcuate nucleus.

### Example 2: HDAC6 Deacetylates LepRb and Reduces Leptin Signaling

It was investigated whether HDAC6 regulates leptin receptor signaling.

### Materials and Methods

It was hypothesized that HDAC6 might interact with leptin receptor b (LepRb). To test this hypothesis, HEK293 cells were transfected with GFP or

HDAC6-myc or LepRb-flag or LepRb-flag and HDAC6-myc expressing plasmids. Subsequently, cells were treated either with vehicle or HDAC6 inhibitor (ACY775) (200 nM for 1 h). Then Flag immunoprecipitation was performed to pull down LepRb and subsequently immunoblot these immune-precipitates with Myc and Flag antibodies.

HEK293 cells were transfected with GFP (as control) or HDAC6 or LepRb or LepRb and HDAC6 together. Subsequently cells were either treated with vehicle or HDAC6 inhibitor (ACY775) (200 nM for 1 h). Flag immunoprecipitation was used to pull down LepRb, followed by Myc and Flag immunoblotting.

HEK293 cells were transfected with the plasmids and were treated with vehicle or ACY775. Subsequently Flag immunoprecipitation was used to pull down LepRb followed by Ac^{Lys} or Flag immunoblotting.

HEK293 cells transfected with the plasmids were pre-treated with leptin (200 ng/ml) for 10 mins, then the media containing leptin was withdrawn and replaced with leptin free media after washing the cells with PBS twice. Subsequently, Flag immunoprecipitation was used to pull down LepRb followed by P^{Tyr} or Flag immunoblotting.

HEK293 cells were transfected with plasmids and treated with leptin (250 ng/ml). STAT3^{Tyr705} phosphorylation together with the control parameters were analyzed with immunoblotting. The ratio of p-STAT3^{Tyr705} to STAT3 signal in the immunoblots was quantified.

Next, a time course experiment was performed to analyze tyrosine phosphorylation of LepRb after removal of leptin. The HEK293 cells were transfected with LepRb and HDAC6 plasmids in the indicated order and then stimulated with leptin (200 ng/ml) for 10 minutes. Subsequently media was changed with non-leptin containing media and tyrosine phosphorylation of LepRb was analyzed 5, 10, 20 and 30 minutes later. While control cells, which did not express HDAC6 maintained the LepRb phosphorylation, HDAC6-expressing cell LepRb phosphorylation started to decay after 20 minutes.

To analyze the effect of HDAC6 expression on the down stream of LepRb signaling, the HEK293 cells were transfected with HDAC6 and/or LepRb expressing plasmids, and the cells stimulated with leptin (200 ng/ml) for the indicated time points and STAT3^{Tyr705} tyrosine phosphorylation (p-STAT3^{Tyr705}) analyzed.

### Results

The results demonstrates that HDAC6 deacetylates LepRb and reduces leptin signaling.

To test whether HDAC6 can deacetylate LepRb, HEK293 cells were transfected with plasmids (LepRb-Flag, HA-p300 and HDAC6-myc). LepRb was immunoprecipitated by Flag antibodies, which was followed by acetyl-lysine (Ac^{Lys}) or immunoblottings. The immunoblots showed that HDAC6 expression leads to deactylation of LepRb.

Next, cells were transfected with plasmids (LepRb-Flag, HA-p300 and HDAC6-myc) and subsequently treated with ACY775 (200 nM/ml) for 0.5, 1, 2 and 3 h. LepRb immunoprecipitation followed by acetyl-lysine immunoblotting documented that specific HDAC6 inhibitor ACY775 can completely block deactylation of LepRb. Next, to investigate whether HDAC6 has any effect on LepRb tyrosine phosphorylation, which is indicative of LepRb activation, HEK 293 cells were transfected with LepRb and HDAC6-Myc plasmids in the indicated order and the cells stimulated with leptin (200 ng/ml) for 10 and 60 minutes. The Flag immunoprecipitates were exposed to phospho-tyrosine (p^{Tyr}) immunoblotting.

It was found that HDAC6 expression did not alter leptin-induced LepRb tyrosine phosphorylation at the early time point (10 min) of leptin stimulation. However, at the later time point (60 min), LepRb tyrosine phosphorylation was almost completely diminished in HDAC6-expressing cells, when compared to the control cells expressing GFP.

It then was found that HDAC6 interacts with LepRb and a specific HDAC6 inhibitor ACY775 (2-[[1-(3-fluorophenyl)cyclohexyl]amino]-N-hydroxy-5-pyrimidinecarboxamide) blocks this interaction. ACY775 is an established HDAC6 inhibitor and increases tubulin acetylation when the cells are treated with this inhibitor. After observing that HDAC6 interacts with LepRb, whether LepRb can be de-acetylated by HDAC6 was investigated. For this purpose, various acetyl transferases were tested to see whether LepRb can be acetylated by one of them. It was found that EIA binding protein p300 (p300) leads to increased acetylation of LepRb.

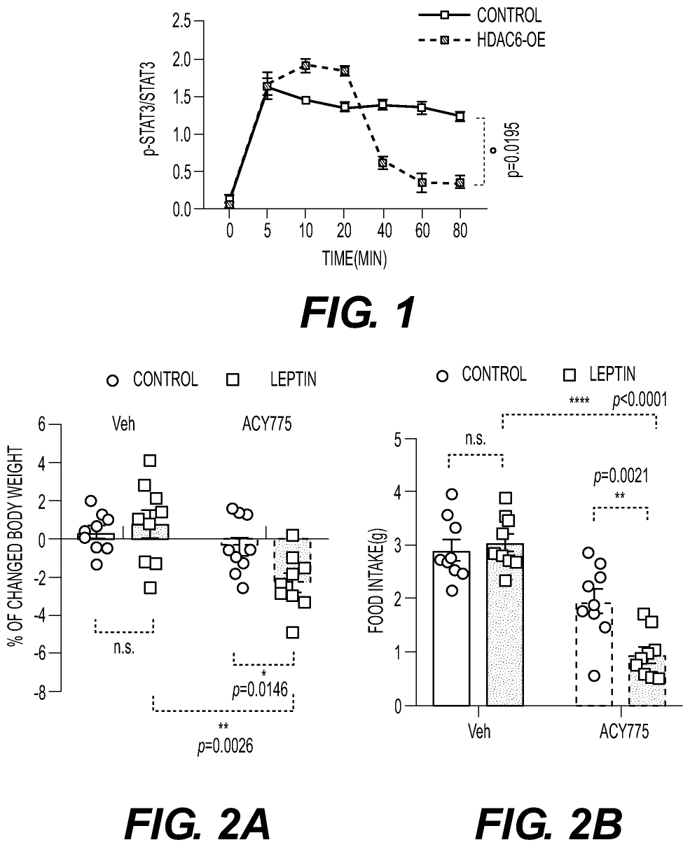

Non-HDAC6 expressing cells maintained the p-STAT3^{Y705} through out the experimental period. However, STAT3^{Tyr705} phosphorylation started to decay around 40 min in the HDAC6-expressing cells. See FIG. 1.

These results show that HDAC6 interacts with LepRb, leading to deacetylation and faster dephosphorylation of the receptor following activation.

### Example 3: Specific HDAC6 Inhibitor ACY775 Increases Energy Expenditure and Reduces RER and Leptin Sensitivity, and Decreases Appetite and Bodyweight in DIO Mice

### Materials and Methods

Diet induced obese (DIO) mice were placed into metabolic cages and received ACY775 (10 mg/kg, i.p., n=7) or vehicle (n=8) once a day for 3 days. Energy expenditure, respiratory exchange ratios (RER; VCO₂/VO₂), ambulatory count (physical activity) from each group of mice were determined. Bar graphs represent average of two dark (8-20 and 32-44 hr) and two light cycles (0-8, 20-32 and 44-48 hr).

DIO mice were pretreated with ACY775 (10 mg/kg/day, i.p.) or vehicle for 3 days, which was followed by co-treatment with ACY775 (or vehicle) and leptin (1 mg/kg, i.p.) (or saline) (n=9 for each group), % change in body weight 15 h after leptin treatment, food intake (g) during the 15h period after leptin treatment. The results in were reproduced in two independent experiments.

DIO mice were pretreated with ACY775 (10 mg/kg/day, i.p.) or vehicle for 3 days, which was followed by a last injection of ACY775 (or vehicle) in the morning of fourth day. 6 hours later mice were injected with saline or leptin (1 mg/kg, i.p.) and hypothalamus were extracted after 45 minutes following the leptin injection. Representative immunoblots for STAT3^{Tyr705} phosphorylation and total STAT3 from the hypothalamus. Quantification of the ratio of p-STAT3^{Tyr705} to total-STAT3 signal in the immunoblots. The results were reproduced in three independent experiments. The results were analyzed hourly.

DIO mice were treated with ACY775 (10 mg/kg/day, i.p.) for 3 weeks. Daily body weight (g) of DIO mice treated with vehicle (n=17) or ACY775 (n=17) during 3 weeks of treatment was determined. Daily food intake (g) during 3 weeks of vehicle or ACY775 treatment was assessed. 24 hours' food intake per mouse during the first week of vehicle or ACY775 treatment was determined. Serum leptin (ng/ml) levels after 3 weeks of vehicle (n=24) or ACY775 (n=24) treatment was assessed. Lean mass (g), fat mass (g) and fat percentage (%) after 3 weeks of vehicle (n=17) or ACY775(n=17) treatment measured by Dual-energy X-ray absorptiometry (DEXA) scan. DEXA scans were performed in two different cohorts. Glucose tolerance test (GTT) after 1 week of vehicle (n=33) or ACY775 (n=26) treated DIO mice was assessed. Area of curve (AOC) analysis for GTT was performed. Blood glucose (mg/dl) levels after 1 week of vehicle (n=17) or ACY775 (n=16) were assessed in treated DIO mice. Insulin tolerance test (ITT) was performed after 2 weeks of vehicle (n=31) or ACY775 (n=21) treatment of DIO mice. Serum insulin (ng/ml) levels were measured after 3 weeks of vehicle (n=22) or ACY775 (n=21) in treated DIO mice. A homeostatic model assessment of insulin resistance (HOMA-IR) was analyzed after 3 weeks of vehicle (n=14) or ACY775 (n=13) treated DIO mice.

Comprehensive Lab Animal Monitoring System (CLAMS) was used to measure metabolic parameters such as energy expenditure (EE) and respiratory exchange ratio (RER) and physical activity levels in Vehicle- and ACY775-treated mice.

Diet induced obese (DIO) mice were pretreated with ACY775 (10 mg/kg/day, i.p.) or vehicle for 3 days, which was followed by co-treatment with ACY775 (or vehicle) and leptin (1 mg/kg, i.p.) (or saline) (n=9 for each group). Mice were evaluated for % change in body weight 15 h after leptin treatment, and food intake (g) during the 15 h period after leptin treatment.

DIO mice were pretreated with ACY775 (10 mg/kg/day, i.p.) or vehicle for 3 days, which was followed by a last injection of ACY775 (or vehicle) in the morning of fourth day. 6 hours later mice were injected with saline or leptin (1 mg/kg, i.p.) and the hypothalamus were extracted after 45 minutes following the leptin injection.

Ac-tubulin activity of HDAC6 inhibitors (ACY775, ACY738 and Ricolinostat) in vitro was tested in HEK293 cells transfected with GFP or HDAC6, then treated with the indicated HDAC6 inhibitors for 30 minutes, as measured by Ac-tubulin immunoblotting in ACY775-treated, ACY738-treated and ACY1215 (ricolinostat)-treated cells.

DIO mice were treated with vehicle or ACY775 (10 mg/kg, i.p., once a day) for 3 days. On the fourth day, 1 h after the beginning of the light cycle, the mice were administered vehicle or ACY775 (10 mg/kg, i.p.) and the conditioned place preference (CPP) assay under the ad libitum feeding condition was utilized. The CPP assay was performed 6 h later under the ad libitum feeding condition (n=10 mice for both the Vehicle- and ACY775-treated groups). CPP assay under a 20-h fasting condition was also performed. DIO mice were treated with vehicle or ACY775 (10 mg/kg, i.p.) once a day and fasted for 15 h after the third injection. On the fourth day, 1 h after the beginning of the light cycle, the mice were administered vehicle or ACY775 (10 mg/kg body weight, i.p.) and the CPP assay was performed 5 h after this injection under the fasting condition (n=10 mice for both the Vehicle- and ACY775-treated groups).

Measurements were made of the total distance that the mice traveled during the CPP test, the average velocity of movement during the assay, the total time the mice spent in the dark chamber during the test; the frequency with which the mice traveled to the dark chamber during the test; the total time the mice spent in the food-paired white chamber; the frequency with which the mice traveled to the food paired white chamber; the total time the mice spent in the food-containing zone within the food-paired side chamber; and the frequency with which the mice traveled to the food-containing zone within the food-paired side chamber during the test.

### Results

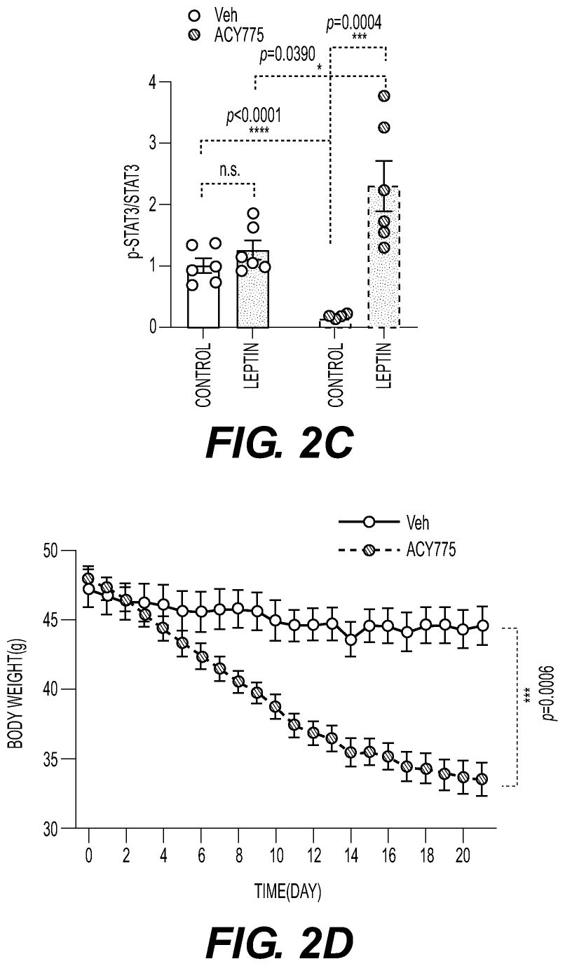

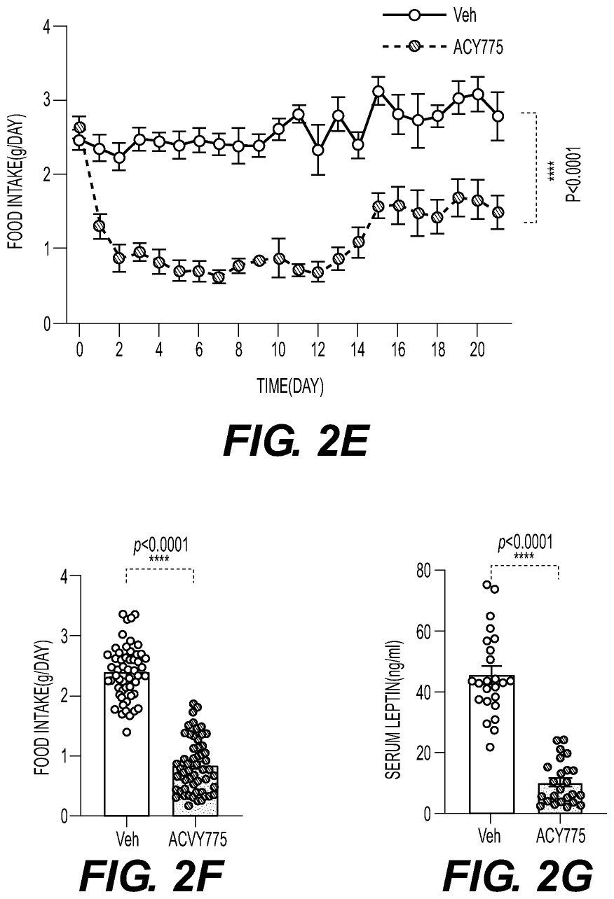

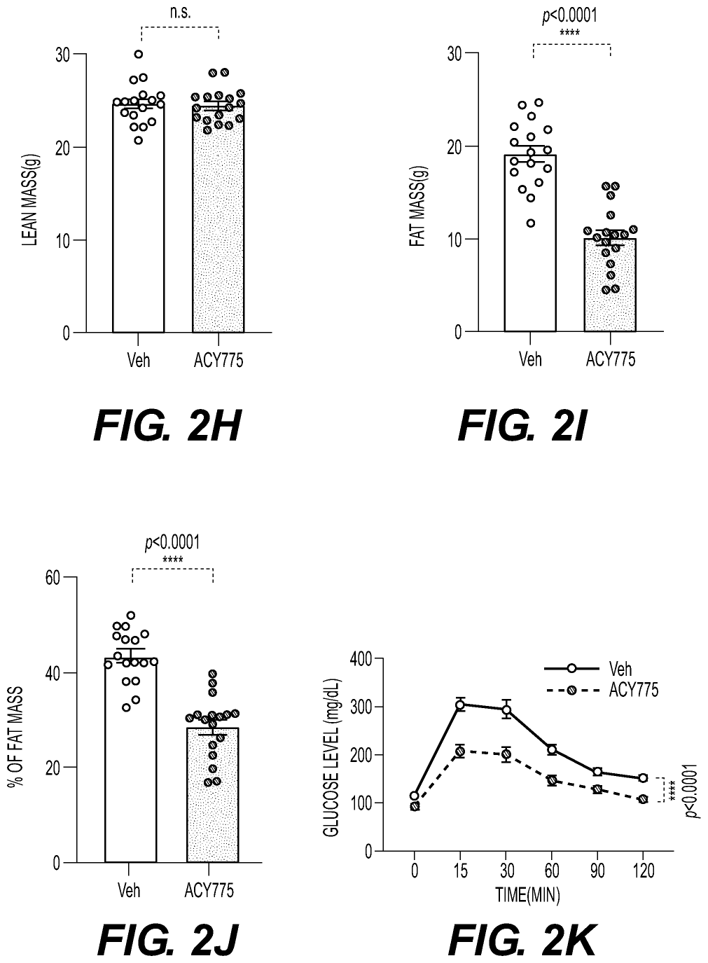

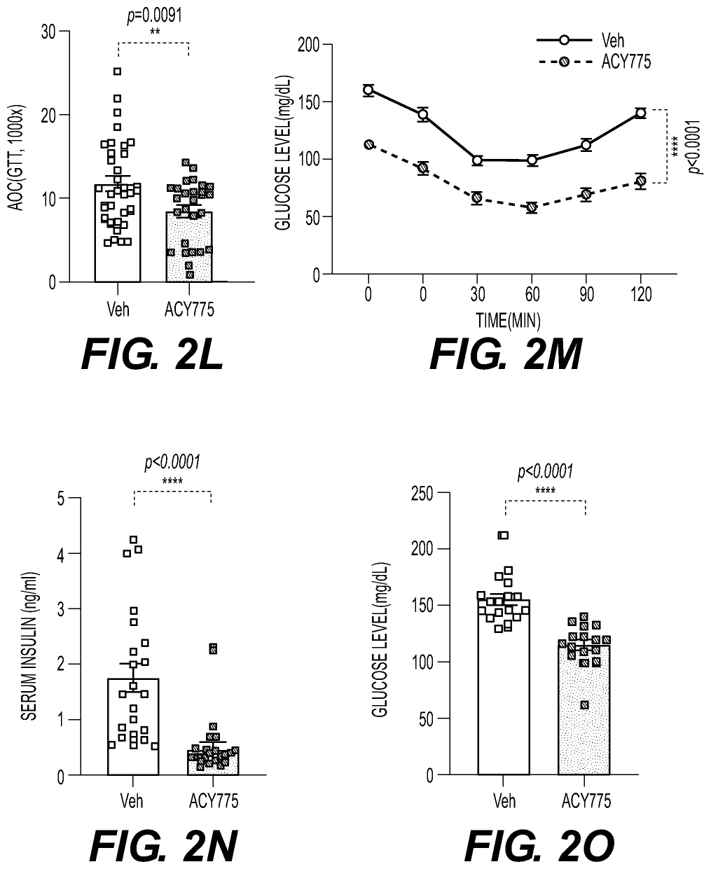

As shown by FIGS. 2A-2O, specific HDAC6 inhibitor (ACY775) increases leptin sensitivity, decreases appetite and bodyweight and improves glucose homeostasis.

To investigate whether HDAC6 inhibition increases leptin sensitivity in vivo, several different experimental methods were used. First, if HDAC6 increases leptin sensitivity, it should acutely potentiate the anorectic and weight-reducing effects of exogenous leptin in DIO mice. The response of the DIO animals to leptin was measured in the presence and absence of ACY775 (10 mg/kg, i.p.).

Administration of leptin to DIO mice did not significantly alter their bodyweight, compared to the control group. ACY775 treatment alone significantly decreased the bodyweight of DIO mice, and administration of leptin to DIO mice that were pre-treated with ACY775 led to a further reduction in bodyweight relative to the ACY775+ Veh group. Furthermore, treatment with leptin alone did not significantly change the food intake of DIO mice. ACY775 treatment alone reduced the food intake of DIO mice. Administration of leptin in the presence of ACY775 demonstrated that ACY775 further augmented the negative effect of leptin on food intake.

Subsequently, the in vivo activation of leptin receptor signaling following ACY775 treatment was investigated. To check whether ACY775 increases hypothalamic leptin sensitivity, it was analyzed how acute ACY775 treatment influences leptin-stimulated STAT3^{Tyr705} phosphorylation in DIO mice at a time when the mice are still obese and hyperleptinemic.

Leptin injection to the vehicle-treated DIO mice did not significantly increase STAT3^{Tyr705} phosphorylation. However, when leptin was administered to DIO mice that were pre-treated with ACY775, the levels of p-STAT3^{Tyr705} in the hypothalamus increased significantly, indicating that treatment of DIO mice with acute ACY775 increases leptin sensitivity.

A single dose of ACY775 or ACY738 significantly reduced the food intake of the DIO mice, without extra-leptin stimulation.

Administration of ACY775 significantly reduced the body weight of DIO mice, from 48.25±0.87 g to 33.53±1.19 g, corresponding to a 29.58%±1.79% decrease in body weight. The body weight of vehicle-treated control mice did not show significant alterations during the experimental period. The food intake of control group was steady through out the trial.

However, ACY775 treatment significantly reduced the food intake from 2.54±0.10 g to 0.85±0.06 g, where the daily food intake stayed in these range during the first two weeks. The average food intake during the first week of treatment documented a highly significant reduction compared to those of control group. Reduction seen in food intake during the first two weeks gradually increased during the third week. This increase in the food intake during the last week of treatment was inversely correlated to the levels of circulating leptin, which was significantly downregulated by the end of the 3-week treatment period.

To determine the lean and fat mass amounts in the whole body of Veh- and ACY775-treated DIO mice, Dual-energy X-Ray absorptiometry (DEXA) scans were performed after 21 days of treatment. ACY775 treatment did not alter the lean mass of DIO mice when compared to the vehicle-treated control mice. However, the total fat mass and fat percentage of ACY775-treated mice was significantly lower than that of vehicle-treated group.

A glucose tolerance test (GTT) was administered after one-week treatment to investigate whether ACY775 improves glucose homeostasis in DIO mice. Treatment with ACY775 significantly improved glucose tolerance relative to the vehicle-treated control group. Consistent with improved glucose tolerance, blood glucose levels were significantly reduced by ACY775. Additionally, insulin tolerance tests (ITT) performed in the third week of treatments revealed significant difference between ACY775-treated mice and those treated with vehicles. ACY775 significantly reduced the circulating insulin levels. In conjunction with improved GTT, reduced blood glucose and insulin levels, the Homeostatic Model Assessment for Insulin Resistance (HOMA-IR) analysis revealed that ACY775 treatment significantly reduced the insulin resistance in DIO mice. ACY775 treatments almost completely abolished hepatic steatosis that is seen in DIO mice.

To investigate whether HDAC6 inhibitors could decrease the food searching behavior in the DIO mice, separate cohorts of mice administered with Vehicle or ACY775 for three days in ad libitum or 20h fasted state were placed into a (food)-conditioned place preference (CPP) assay during the light cycle. The mouse-center-based representative traces of vehicle-treated mice showed more activity in the food zone (right-down corner of the white chamber) when compared to the ACY775-treated mice at both fed and fasting conditions, which indicated that the vehicle-treated mice show a higher interest for food compared to the ACY775-treated mice. Furthermore, determination of locomotion and velocity depending on mouse-nose-based analysis has shown that both vehicle- and ACY775-treated DIO mice had similar levels of locomotion and velocity levels both in fed or fasting conditions. There were also no significant differences in cumulative duration that the mice spent in the dark side chambers, which contained the mimic food. Appearance frequency of the mice within the dark side chambers were also similar for both Veh- and ACY75-treated DIO mice at both fed or fasting conditions. ACY775-treated mice, compared to the vehicle-treated mice, spent significantly shorter time and had lower frequency of appearance in the food-containing white side chambers in both fed and fasting conditions. Notably, ACY775-treated mice, compared to the vehicle group, also spent significantly shorter time and displayed lower frequency of appearance in the food zone in both fed and fasting conditions in the white side chambers.

In summary, ACY775 increases energy expenditure and reduce reduction of energy expenditure (“RER”) in DIO mice. Leptin is reported to block RER even though it reduces food intake. Therefore, despite severely reducing food intake and body weight, a leptin sensitizer should enhance/maintain energy expenditure and lead to the utilization of fat as the main energy source. EE of the ACY775-treated mice was higher in the both dark and light cycle compared to the vehicle-treated controls. Also, RER values were significantly decreased in ACY775-treated DIO mice in both the dark and light cycles, indicating more utilization of fat as energy source in the body. Physical activity level during the dark and light cycles were not different between ACY775- and vehicle-treated groups.

HDAC6 Inhibitor (ACY775) shows decreased appetite and decreased foraging behavior in DIO mice), as measured using the Conditioned place preference (CPP) assay under the ad libitum feeding condition.

To investigate whether HDAC6 inhibitors could decrease the food searching behavior in the DIO mice, separate cohorts of mice administered with Vehicle or ACY775 for three days in ad libitum or 20 h fasted state were placed into a (food)-conditioned place preference (CPP) assay during the light cycle.

The mouse-center-based representative traces of vehicle-treated mice showed more activity in the food zone when compared to the ACY775-treated mice at both fed and fasting conditions, which indicated that the vehicle-treated mice show a higher interest for food compared to the ACY775-treated mice. Determination of locomotion and velocity depending on mouse-nose-based analysis showed that both vehicle- and ACY775-treated DIO mice had similar levels of locomotion and velocity levels both in fed or fasting conditions. There were no significant differences in cumulative duration that the mice spent in the dark side chambers, which contained the mimic food. Appearance frequency of the mice within the dark side chambers were also similar for both Veh- and ACY75-treated DIO mice at both fed or fasting conditions. ACY775-treated mice, compared to the vehicle-treated mice, spent significantly shorter time and had lower frequency of appearance in the food-containing white side chambers in both fed and fasting conditions. Notably, ACY775-treated mice, compared to the vehicle group, also spent significantly shorter time and displayed lower frequency of appearance in the food zone in both fed and fasting conditions in the white side chambers.

### Example 4: Specific HDAC6 Inhibitor (ACY738) Increase Leptin Sensitivity, Decreases Appetite and Bodyweight in DIO Mice

### Methods and Materials

DIO mice pretreated with ACY738 (50 mg/kg/day, i.p.) or vehicle for 3 days, which was followed by co-treatment with ACY738 (or vehicle) and leptin (1 mg/kg, i.p.) (or saline) (n=14 for each group). % change in body weight 15 h after leptin treatment, food intake (g) during the 15 h period after leptin treatment were measured.

DIO mice pretreated with ACY738 (50 mg/kg/day, i.p.) or vehicle 3 days, which was followed by a last injection of ACY738 or vehicle in the morning of fourth day. 6 hours later mice were injected with saline or leptin (1 mg/kg, i.p.) and hypothalamus were extracted after 45 minutes following the leptin injection. Immunoblots for STAT3^{Tyr705} phosphorylation and total STAT3 from the hypothalamus were made to quantify the ratio of p-STAT3^{Tyr705} to total-STAT3 signal in the immunoblots. The results were reproduced in two independent experiments.

DIO mice were injected with HDAC6 inhibitors (ACY775, 10 mg/kg, i.p. or ACY738, 50 mg/kg, i.p., n=3 cages with 3 mouse/cage for each group) after 24 h fasting. Food intake was analyzed hourly until 3:00 am. DIO mice were treated with ACY738 (50 mg/kg, i.p.) for 3 weeks. Daily body weight (g) of DIO mice treated with vehicle (n=27) or ACY738 (n=24) during the 3 weeks of treatment period was measured, as was daily food intake (g) during 3 weeks of vehicle or ACY738 treatment and 24 hours' food intake per mouse during the first week of vehicle or ACY738 treatment. Serum leptin (ng/ml) levels after 3 weeks of vehicle (n=26) or ACY738 (n=25) treatment, lean mass (g); fat mass (g) and fat percentage (%) were assessed after 3 weeks of vehicle (n=23) or ACY738(n=23) treatment measured by DEXA scan. DEXA scans were performed in two different cohorts. GTT was measured after 1 week of vehicle (n=23) or ACY738 (n=24) treated DIO mice, and AOC analysis for GTT performed. Blood glucose (mg/dl) levels were measured after 1 week of vehicle (n=27) or ACY738 (n=23) treated DIO mice. ITT was determined after 2 weeks of vehicle (n=27) or ACY738 (n=22) treated DIO mice. Serum insulin (ng/ml) level was measured after 3 weeks of vehicle (n=25) or ACY738 (n=25) treated DIO mice. HOMA-IR analysis after 3 weeks of vehicle (n=14) or ACY738 (n=13) treated DIO mice, and H&E staining of the liver sections from 3 weeks of vehicle or ACY738 treated DIO mice were assessed.

To further investigate whether HDAC6 inhibitors could block appetite in the fasted DIO mice, 24 h-fasted DIO mice were injected with a single dose of ACY775 (10 mg/kg) or ACY738 (50 mg/kg) at the beginning of dark cycle.

### Results

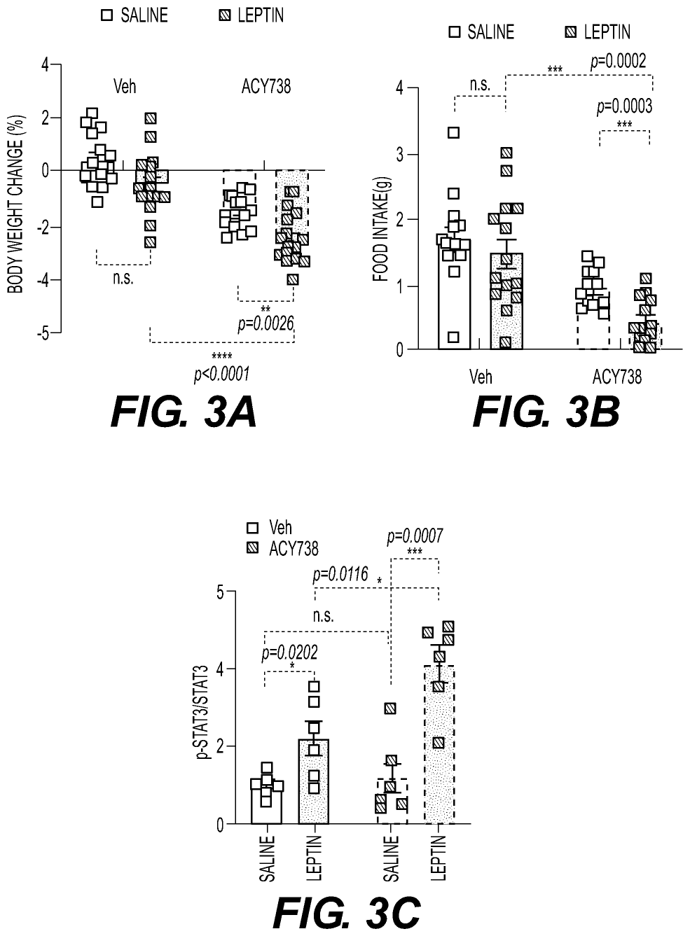

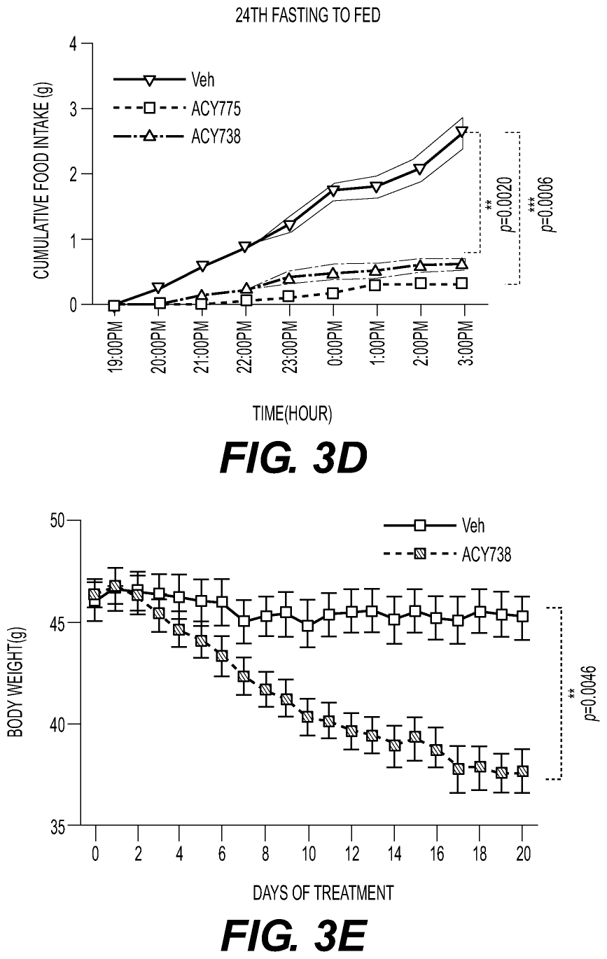

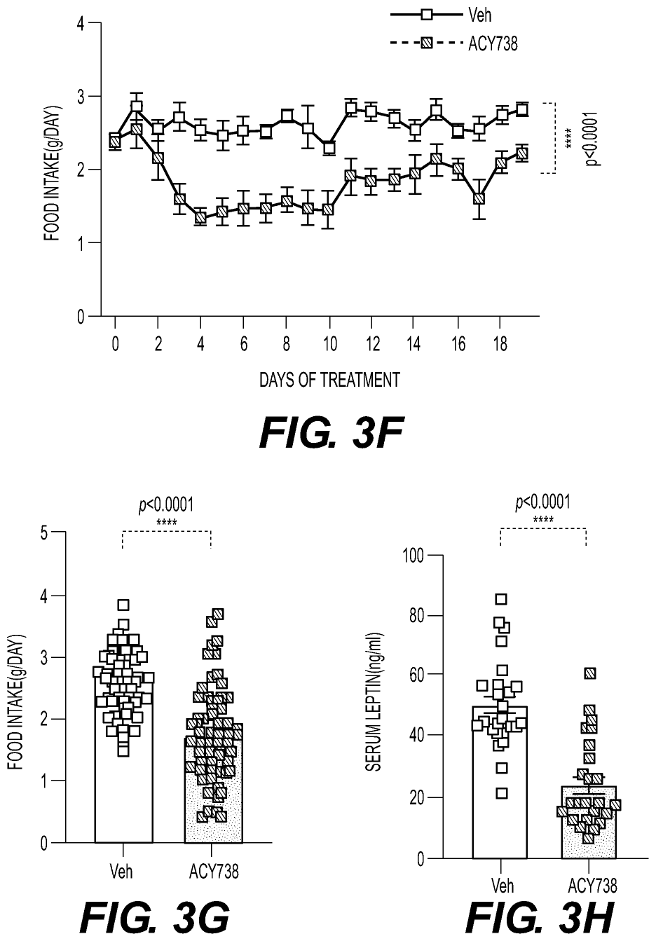

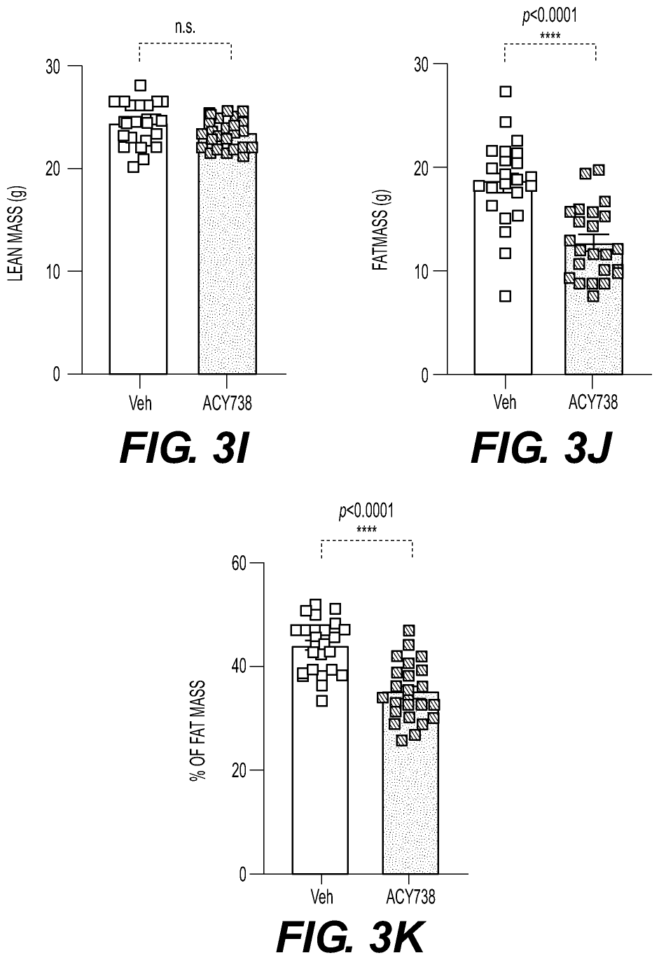

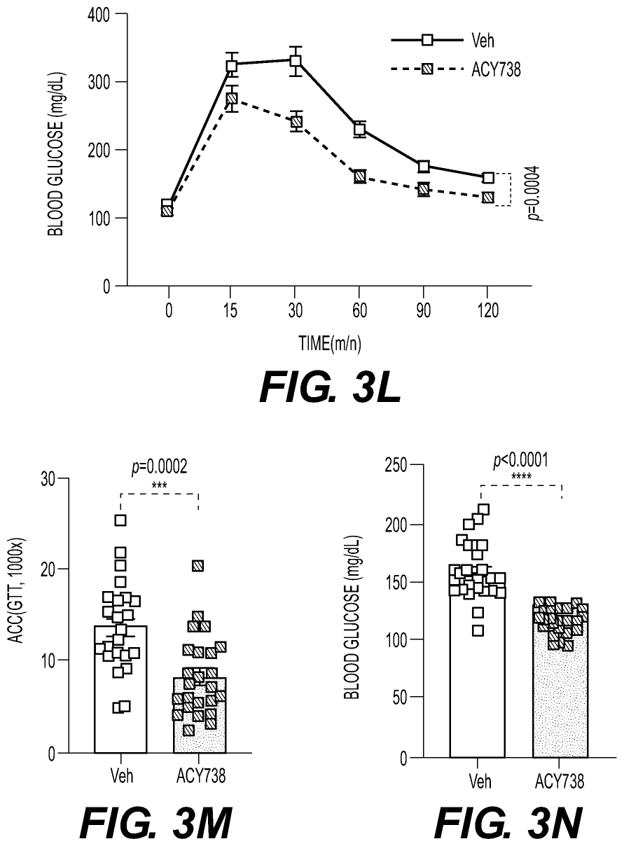

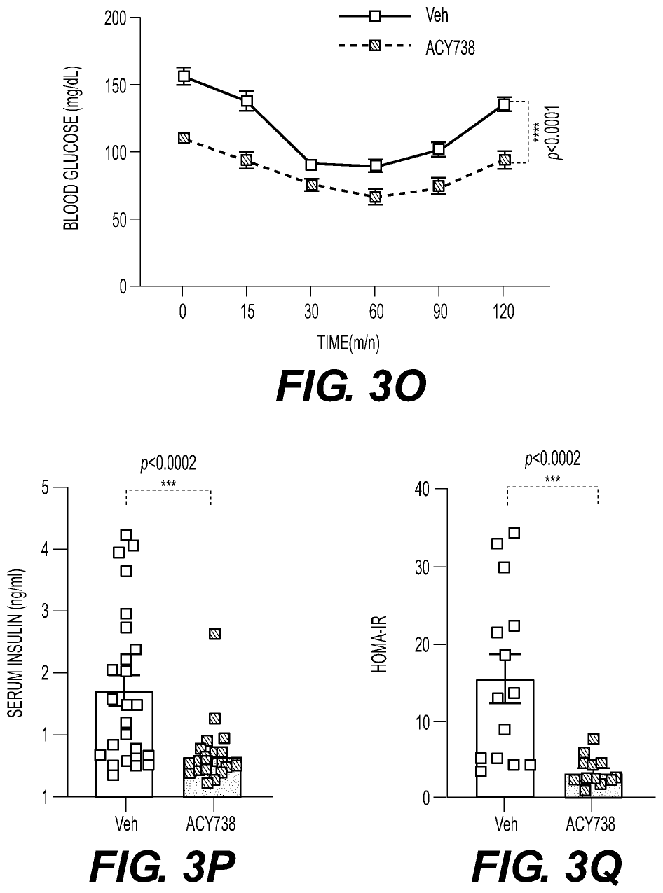

The results are shown in FIG. 3A-3P.

HDAC6 inhibitor ACY738 decreases appetite in DIO mice, as shown using the CPP assay under the ad libitum feeding condition: DIO mice were treated with vehicle or ACY738 (50 mg/kg, i.p., once a day) for 3 days. On the fourth day, 1 h after the beginning of the light cycle, the mice were administered vehicle or ACY738 (50 mg/kg, i.p.) and the CPP assay was performed 6 h later under the ad libitum feeding condition (n=10 for each groups). The CPP assay was also used under a 20-h fasting condition: DIO mice were treated with vehicle or ACY738 (50 mg/kg, i.p.) once a day and fasted for 15 h after the third injection. On the fourth day, 1 h after the beginning of the light cycle, the mice were administered vehicle or ACY738 (50 mg/kg body weight, i.p.) and the CPP assay was performed 5 h after this injection under the fasting condition (n=10 for each group.

It was determined if ACY738 could create similar effects on leptin sensitivity as ACY775 does. ACY738 treatment, compared to vehicle treatment, significantly augmented the acute leptin-induced reduction in body weight and food intake. Similarly, the ratio of leptin stimulated hypothalamic pSTAT3^{Tyr705}/STAT3 ratio in mice treated with ACY738 (50 mg/kg, i.p) for three days also substantially increased when compared to the vehicle-treated mice.

Even a single dose of ACY775 or ACY738, compared to vehicle treatment, significantly reduced/blocked the food intake of the fasted DIO mice.

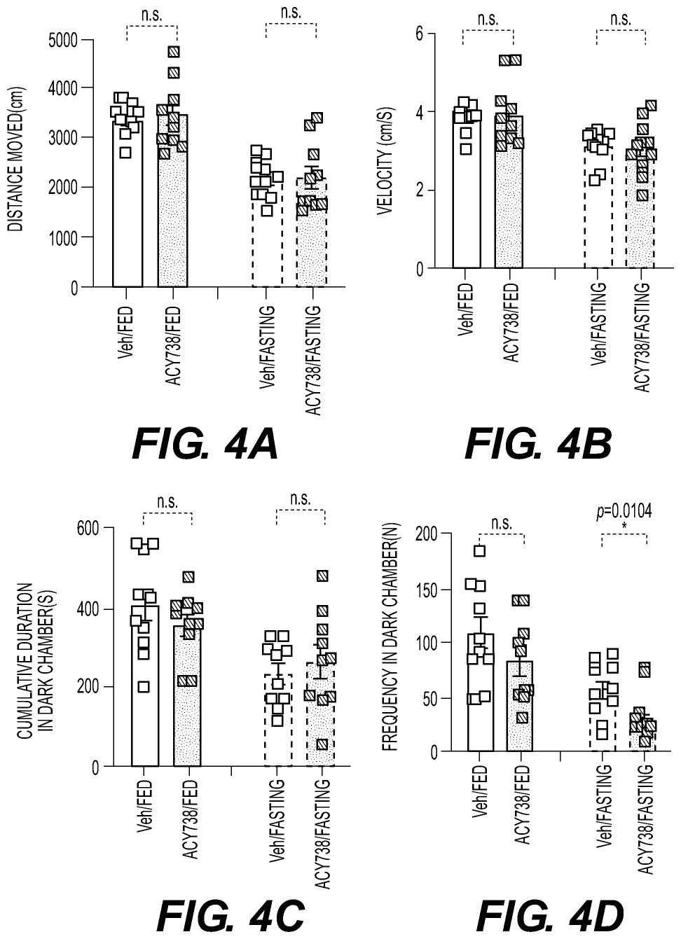

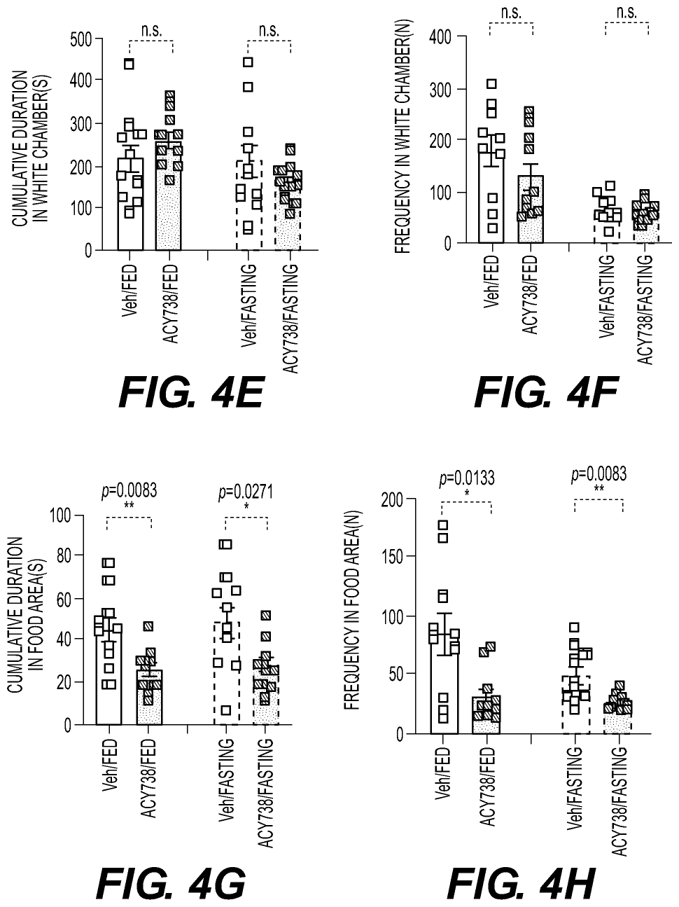

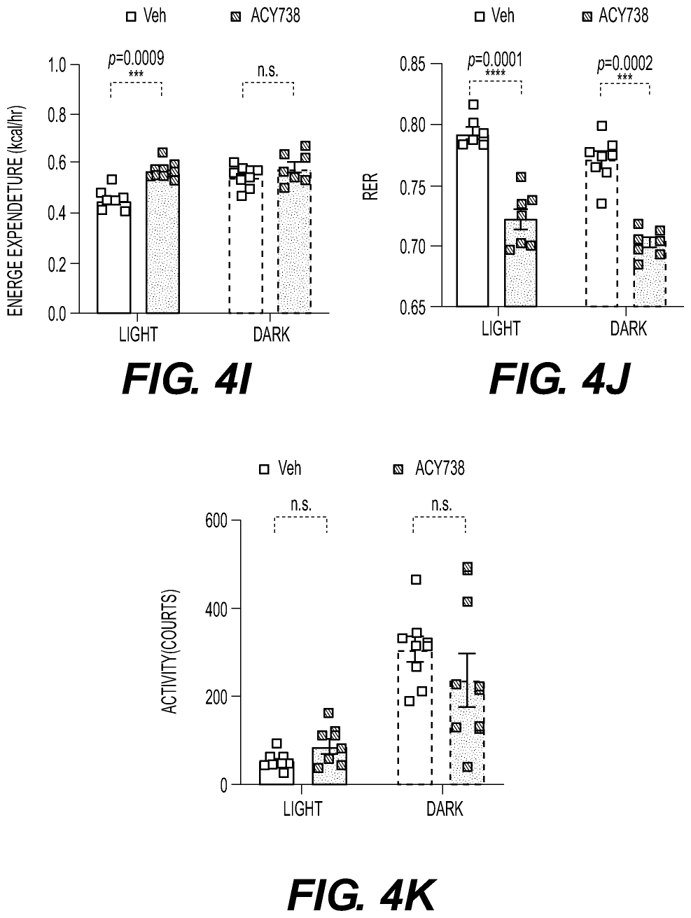

HDAC6 Inhibitor (ACY738) increases energy expenditure and ambulatory count (physical activity) and reduces RER in DIO mice. As shown in FIGS. 4I-4K, HDAC6 inhibitor ACY738 blocks appetite in 24 h-fasted DIO mice injected with a single dose of ACY775 (10 mg/kg) or ACY738 (50 mg/kg) at the beginning of dark cycle.

These results show that another specific HDAC6 inhibitor, ACY738, increases leptin sensitivity, and reduces bodyweight. It was confirmed that ACY738 inhibits the HDAC6 activity by analyzing tubulin acetylation after ACY738 treatment.

Next, it was investigated whether ACY738, similar to the ACY775, could act as an anti-obesity agent when administered to DIO mice in a chronic manner. DIO mice treated with vehicle or ACY738 (50 mg/kg, i.p., once a day) for 21 days.

Administration of ACY738 significantly reduced the body weight of DIO mice, from 46.43±0.74 g to 37.74±1, corresponding to a 20.21%±1.32% decrease in body weight. The body weight of vehicle-treated control mice did not significantly change during the experimental period. The food intake of control group was steady throughout the treatment period. However, ACY738 treatment significantly decreased the food intake from 2.35±0.10 g to 1.32±0.05 g, where the daily food intake stayed in these range over the course of treatment. The average food intake of the drug-treated group during the first week of treatment was severely reduced compared to those of control group. As seen after ACY775 treatment, ACY738 significantly decreased circulating leptin levels, total body fat mass and percentage of body mass from fat at end of the 3-week treatment period. There were no change in the lean body mass of both groups.

Next, ACY738's effect on glucose homeostasis and insulin sensitivity was evaluated. GTT performed after one week of ACY738 treatment revealed a significant improvement of glucose homeostasis. Blood glucose levels were significantly reduced by ACY738 treatment. Like ACY775, ACY738 treatment also improved insulin tolerance and reduced insulin levels as well as ameliorated insulin resistance. Finally, ACY738 treatment also improved hepatic steatosis.

To further confirm HDAC6 inhibitors could decrease the food searching behavior in the DIO mice, a CPP assay was performed in another separate cohort of mice administered with ACY738 for three days in ad libitum or 20 h fasted state. Similar to the case of ACY775 treatment, the representative traces of vehicle-treated mice showed a higher activity in the food zone when compared to the ACY738-treated mice at both fed and fasting conditions, which indicated that the vehicle-treated mice show a higher interest for food compared to the ACY738-treated mice. Determination of locomotion and velocity depending on mouse-nose-based analysis showed that both Veh- and ACY738-treated DIO mice had similar levels of locomotion and velocity levels both in fed or fasting conditions. There were also no significant differences in cumulative duration that the mice spent in the dark side chambers, which contained the mimic food. Appearance frequency of the mice within the dark side chambers were also similar for both Veh- and ACY738-treated DIO mice at fed conditions, but lower for ACY738-treated DIO mice at fasted conditions. Unlike ACY775 treated mice, ACY738-treated mice, compared to the vehicle-treated mice, spent similar time and frequency of appearance in the food-containing white side chambers in both fed and fasting conditions. Interestingly, ACY738-treated mice, compared to the vehicle group, spent significantly shorter time and displayed lower frequency of appearance in the food zone in both fed and fasting conditions in the white side chambers.

To investigate whether ACY738, like ACY775, enhances energy expenditure, metabolic parameters were measured for Vehicle- and ACY738-treated DIO mice. Notably, ACY738-treated mice have a higher energy expenditure in light cycle, though without significant difference in dark cycle when compared to vehicle-treated mice, Similar with ACY775, RER values were significantly decreased in ACY738-treated DIO mice in both the dark and light cycles, indicating more utilization of fat as energy source in the body.

Physical activity level during the dark and light cycles were not different between ACY738- and vehicle-treated groups.

### Example 5: HDAC6 Inhibitors Do Not Reduce Appetite or Body Weight nor Alter Glucose or Insulin Levels in Lean Mice

### Materials and Methods

WT lean mice were treated with vehicle (n=20) or ACY775 (10 mg/kg, i.p., once a day, n=22) or ACY738 (50 mg/kg, i.p., once a day, n=20) for 3 weeks. Daily body weight (g) of WT lean mice treated with Veh, or ACY775 or ACY738 during 3 weeks of treatment period was measured. Daily food intake (g) during 3 weeks of treatment. Serum leptin (ng/ml) levels were measured after 3 weeks of treatment. Lean mass (g); fat mass (g) and fat percentage (%) after 3 weeks of treatment were measured by DEXA scan. GTT was measured after 1 week of treatment. AOC analysis for GTT was performed. Blood glucose (mg/dl) level was measured after 1 week of treatment. ITT was measured after 2 weeks of treatment. Serum insulin (ng/ml) levels were measured after 3 weeks of treatment.

### Results

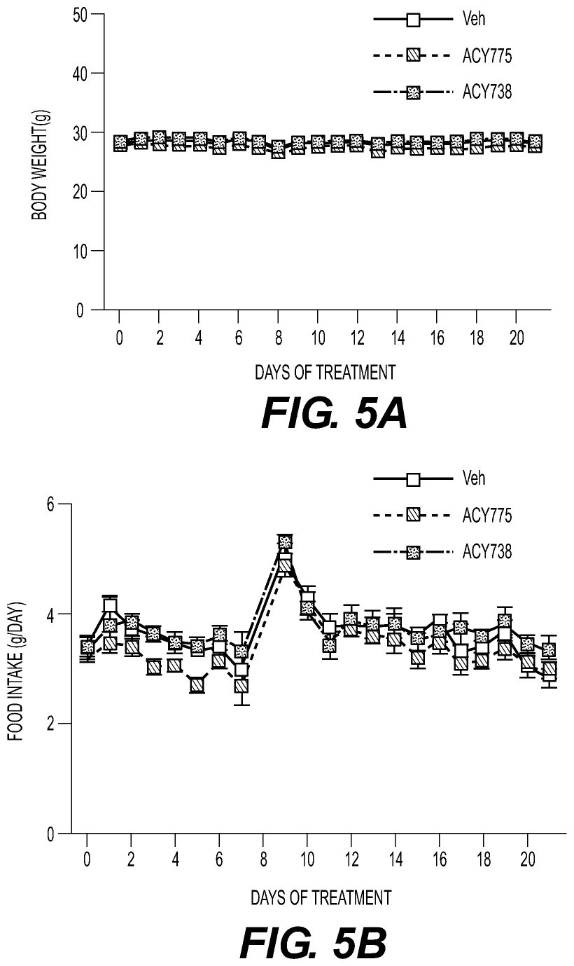

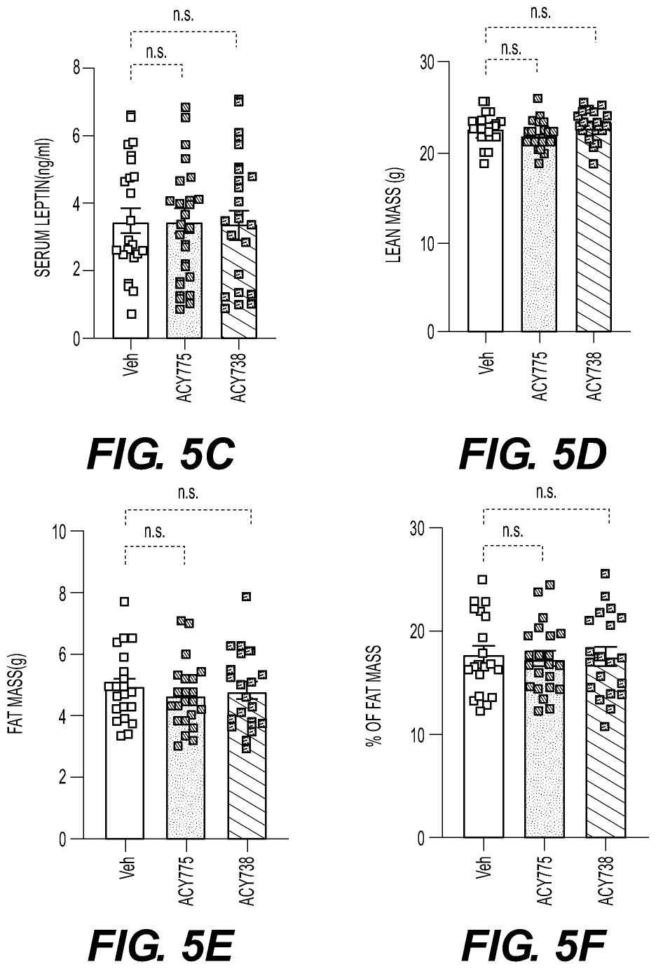

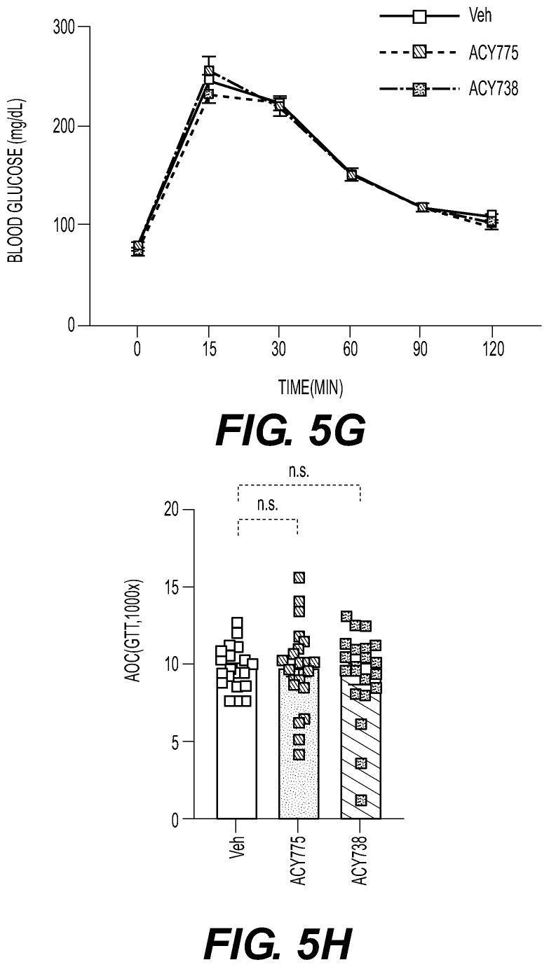

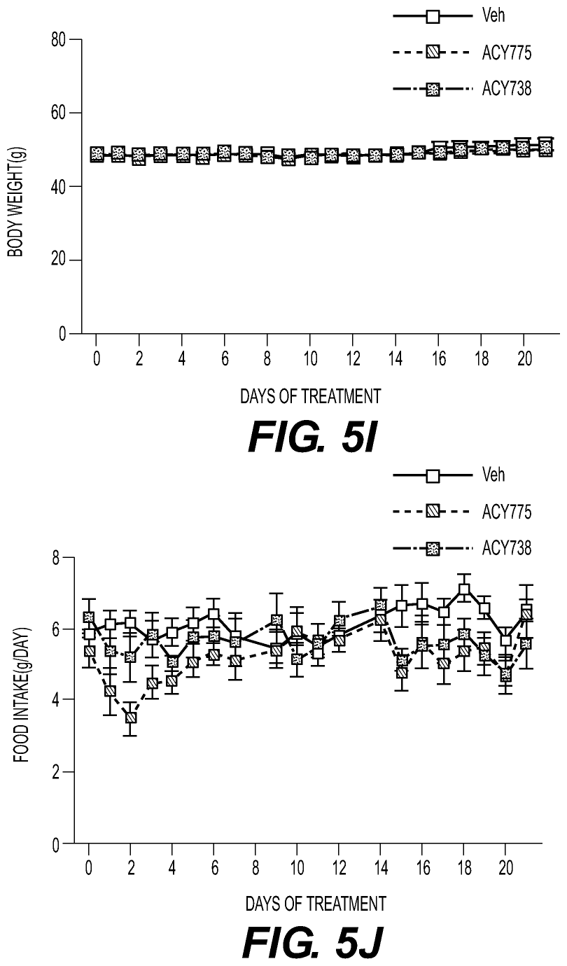

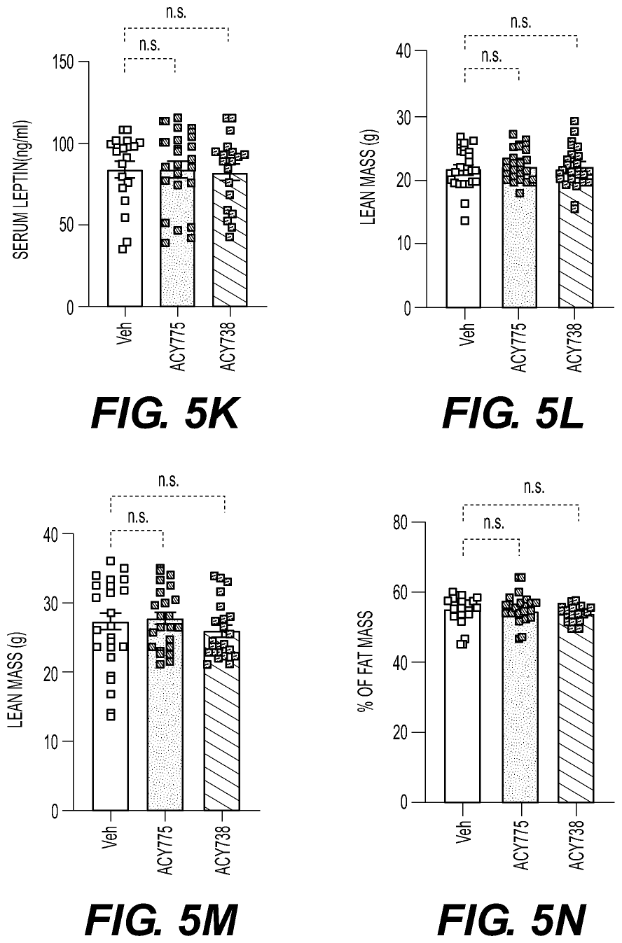

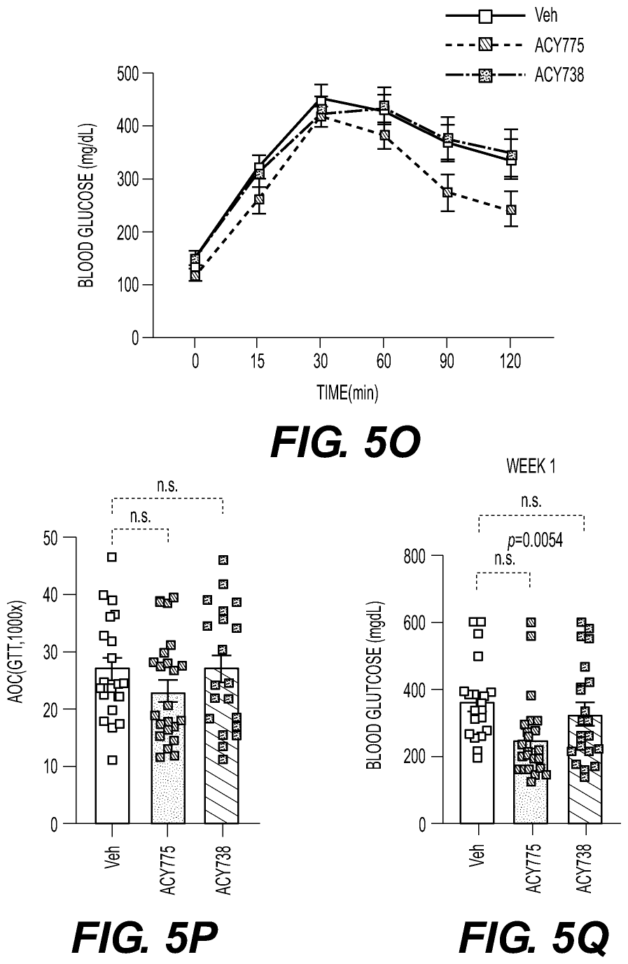

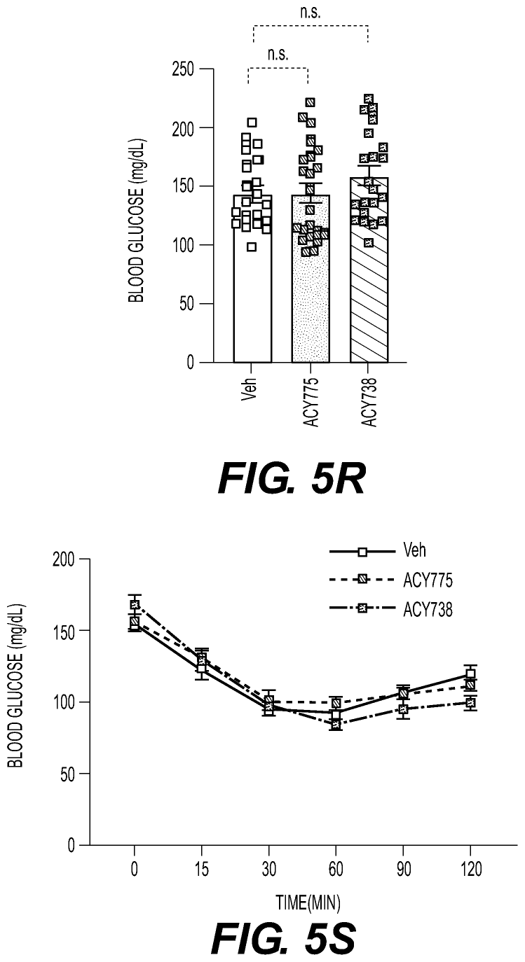

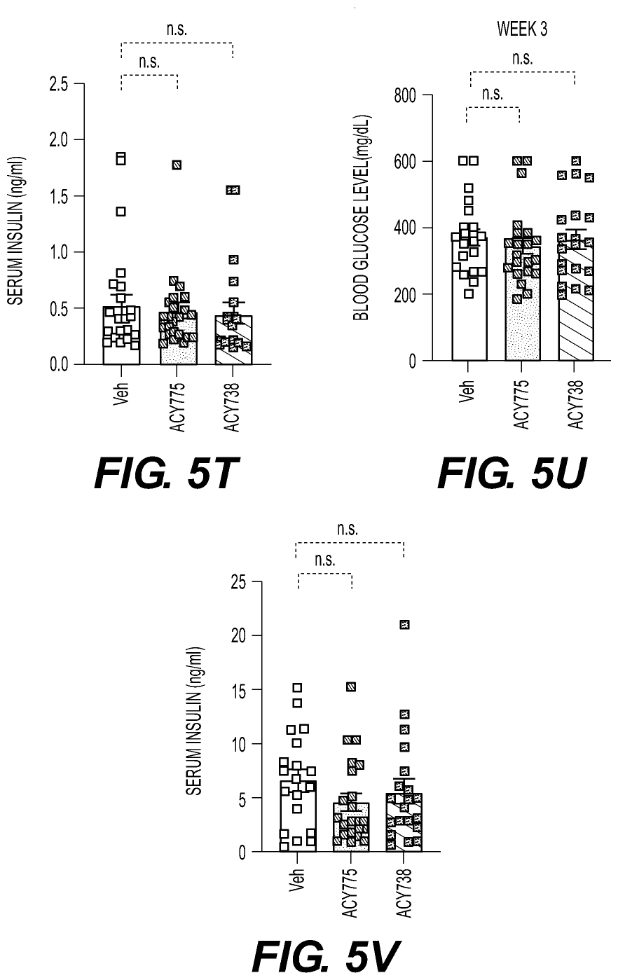

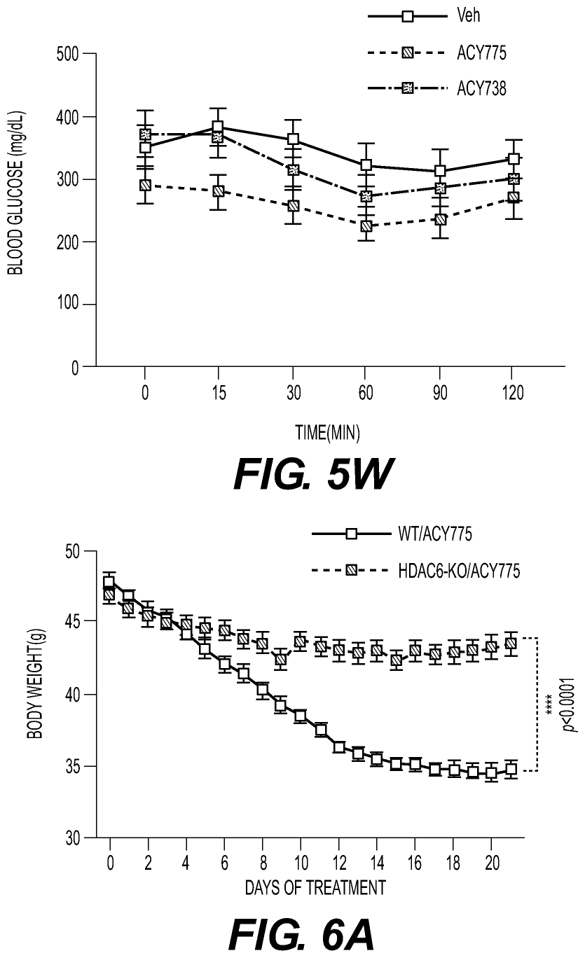

The results comparing ACY738 and ACY775 shown in FIGS. 5A-5H demonstrate that HDAC6 inhibitors do not affect lean mice's appetite, glucose or insulin levels or bodyweight.

As opposed to DIO mice, lean mice have very low levels of circulating leptin levels, and leptin sensitizers do not affect the body weight and food intake of the lean mice. Consistent with acting as leptin sensitizers, neither ACY775 nor ACY738 affected the body weight of lean mice during three weeks of treatments at doses that were otherwise robustly effective in DIO mice. Furthermore, the daily food intake was similar between mice treated with vehicle or HDAC6 inhibitors (ACY775 or ACY738). Consistently, plasma leptin levels of lean mice were not altered by ACY738 or ACY775 treatment. We also did not detect any alterations in the lean mass, total fat mass, or fat percentage of lean mice following three weeks of treatments with either ACY738 or ACY775.

To evaluate whether HDAC6 inhibition improves glucose homeostasis in lean mice, the mice were treated with vehicle and ACY738 or ACY775 for one week and then GTT performed. The disposal of glucose from circulation was not different between Vehicle-, ACY738- or ACY775-treated lean mice. Blood glucose levels were not affected by ACY738 or ACY775. ITT was performed following two weeks of ACY738 or ACY775 treatment and no significant difference in insulin sensitivity and circulating insulin levels were observed between Veh-, ACY738- or ACY775-treated lean mice. Furthermore, the liver morphology was similar between the three groups at the end of treatment.

Together, these findings show that HDAC6 inhibition does not affect body weight, food intake, or glucose homeostasis in lean mice, indicating that hyperleptinemia is required for anti-obesity function of HDAC6 inhibitors.

### Example 6: HDAC6 Inhibitors are Ineffective in db/db Mice

### Materials and Methods

Db/db mice were treated with vehicle (n=20) or ACY775 (10 mg/kg, i.p., once a day, n=22) or ACY738 (50 mg/kg, i.p., once a day, n=20) for 3 weeks. Daily body weight (g) of db/db mice treated with Veh, or ACY775 or ACY738 was measured during 3 weeks of treatment period. Daily food intake (g), Serum leptin (ng/ml), lean mass (g), fat mass (g) and fat percentage (%) were measured after 3 weeks of treatment measured by DEXA scan. GTT was determined after 1 week of treatment. AOC analysis for GTT was performed and Blood glucose (mg/dl) levels were measured after 1 week of treatment. Blood glucose (mg/dl) levels and Serum insulin (ng/ml) levels were measured after 3 weeks of treatment. ITT was assessed after 2 weeks of treatment. H&E staining of the liver sections was conducted after 3 weeks of treatment.

### Results

HDAC6 inhibitors do not reduce bodyweight or blood glucose of db/db mice, as shown by FIGS. 5I-5W. HDAC6 inhibitors are ineffective in db/db mice. Db/db mice were treated with vehicle (n=20) or ACY775 (10 mg/kg, i.p., once a day, n=22) or ACY738 (50 mg/kg, i.p., once a day, n=20) for 3 weeks. FIG. 5I, Daily body weight (g) of db/db mice treated with Veh, or ACY775 or ACY738 during 3 weeks of treatment period. FIG. 5J, Daily food intake (g) during 3 weeks of treatment. FIG. 5K, Serum leptin (ng/ml) levels after 3 weeks of treatment. FIG. 5L, lean mass (g) FIG. 5M, fat mass (g) and FIG. 5N, fat percentage (%) after 3 weeks of treatment measured by DEXA scan. FIG. 5O, GTT after 1 week of treatment. FIG. 5P, AOC analysis for GTT performed in (FIG. 5O). FIG. 5Q, Blood glucose (mg/dl) levels after 1 week of treatment. FIG. 5U, Blood glucose (mg/dl) levels after 3 weeks of treatment. FIG. 5V, Serum insulin (ng/ml) levels after 3 weeks of treatment. 5W, ITT after 2 weeks of treatment. The results in (FIGS. 5I-Q and 5U-W.) were reproduced in two independent experiments.

Db/db mouse model, which is obese, hyperleptinemic but devoid of LepRb action, is used to investigate leptin sensitizers. db/db obese mice were treated with Vehicle, ACY738 (50 mg/kg, i.p, once a day) or ACY775 (10 mg/kg, i.p, once a day) for three weeks. Vehicle-treated db/db mice did not lose weight but gained a small amount of weight during the course of the experiment. Importantly, treatment of db/db mice with HDAC6 inhibitors also did not reduce bodyweight, and the mice gained similar amounts of weight as did the vehicle-treated mice (FIG. 5I); in fact, at the end of the treatment period, the body weight of db/db mice was 3.82%±0.99% heavier than their initial body weight for ACY775-treated group (FIG. 5I), and 2.53%±0.97% heavier for ACY738 treated group. The food intake of db/db mice was not affected by ACY738, but was transitionally reduced by ACY775 treatment during the first 2 days of treatment which were quickly recovered to the levels of vehicle-treated db/db mice (FIG. 5J). Moreover, neither of the HDAC6 inhibitors reduced plasma leptin levels, the amount of lean mass, fat mass, or fat mass percentage in db/db mice (FIG. 5K-5N).

To investigate the effect of HDAC6 inhibitors on glucose homeostasis in db/db mice, GTT was performed after one week of treatments with Veh, ACY738 or ACY775. The results showed no difference in glucose tolerance among Veh-, ACY738- or ACY775-treated groups (FIGS. 5O and 5P). The fasting blood glucose was not affected by ACY738, but was lower in ACY775-treated mice than vehicle-treated mice after the first week of treatment (FIG. 5Q). However, the lower blood glucose from ACY775 treatment in early time was completely diminished at the end of treatment period (FIG. 5U). Plasma insulin levels did not show any significant alterations in ACY775- and ACY738-treated db/db mice when compared to the control db/db group (FIG. 5V). ITT revealed no significant difference in blood glucose levels between vehicle-, ACY738- or ACY775-treated groups following the insulin injection (FIG. 5W). ACY775 or ACY738 treatment did not improve hepatic steatosis in the db/db mice. Thus, these findings indicates that HDAC6 inhibition has no or minimal effects on the db/db mice' bodyweight, food intake, or glucose homeostasis, suggesting that intact leptin signaling is required for the anti-obesity function of HDAC6 inhibitors.

### Example 7: Tubastatin A, Contrary to Specific HDAC6 Inhibitors (ACY775 & ACY738), Reduces Bodyweight and Food Intake in DIO HDAC6 Knock Out and DIO WTmice

### Materials and Methods

DIO WT or HDAC KO mice were treated with specific HDAC6 inhibitors (ACY775 and ACY738) and Tubastatin A for 3 weeks and the effects on food intake, lean mass, fat mass, body weight, blood glucose, and serum insulin were assessed.

### Results

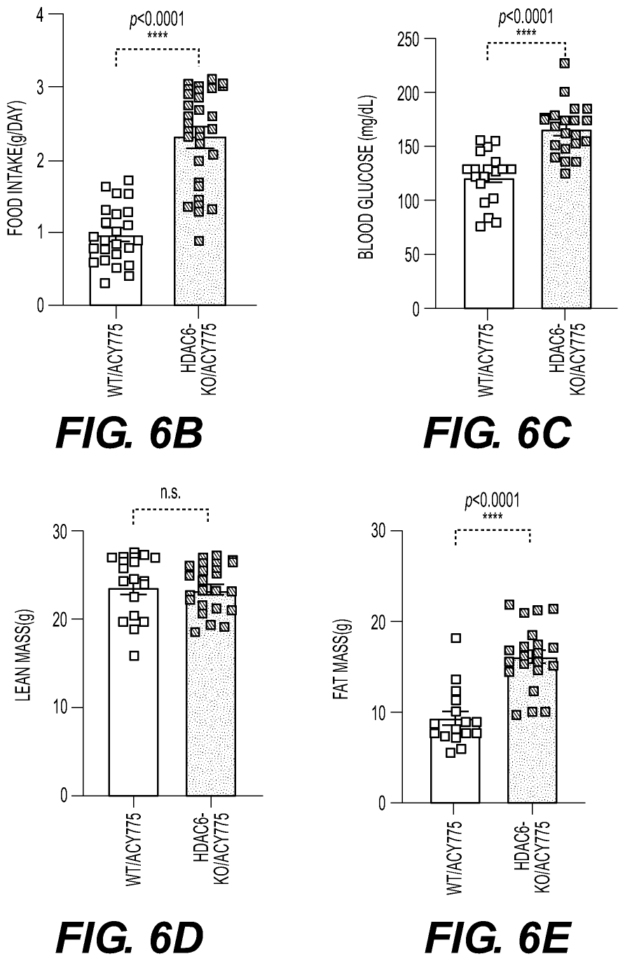

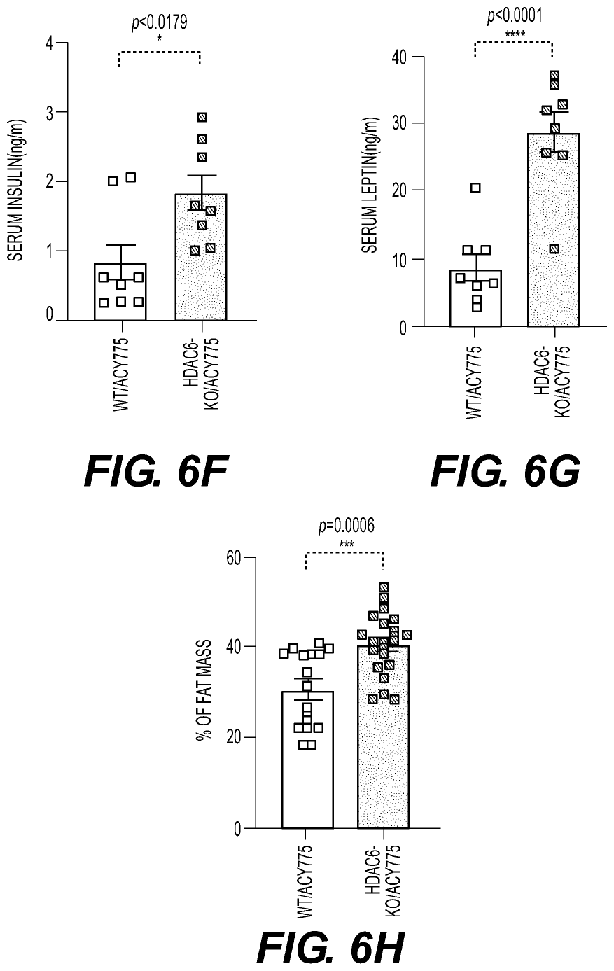

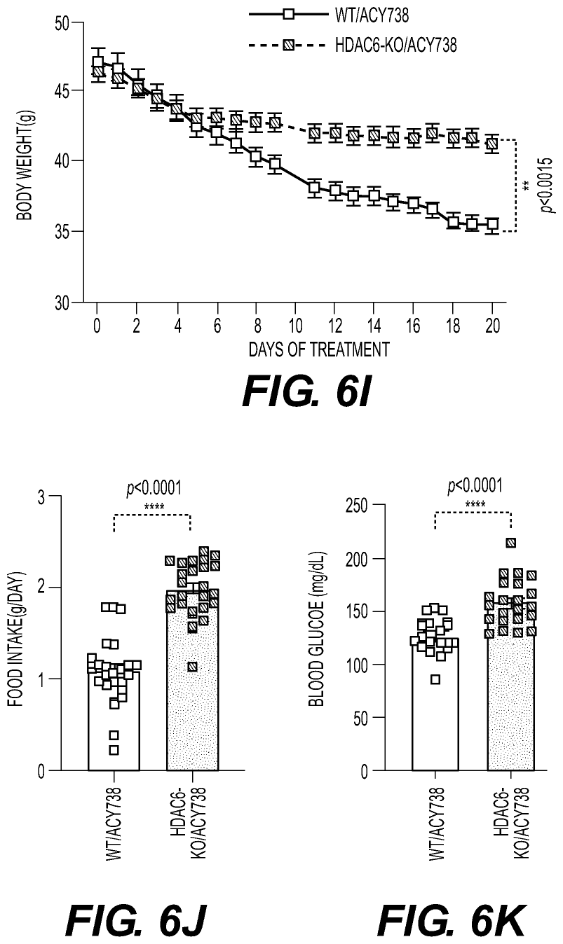

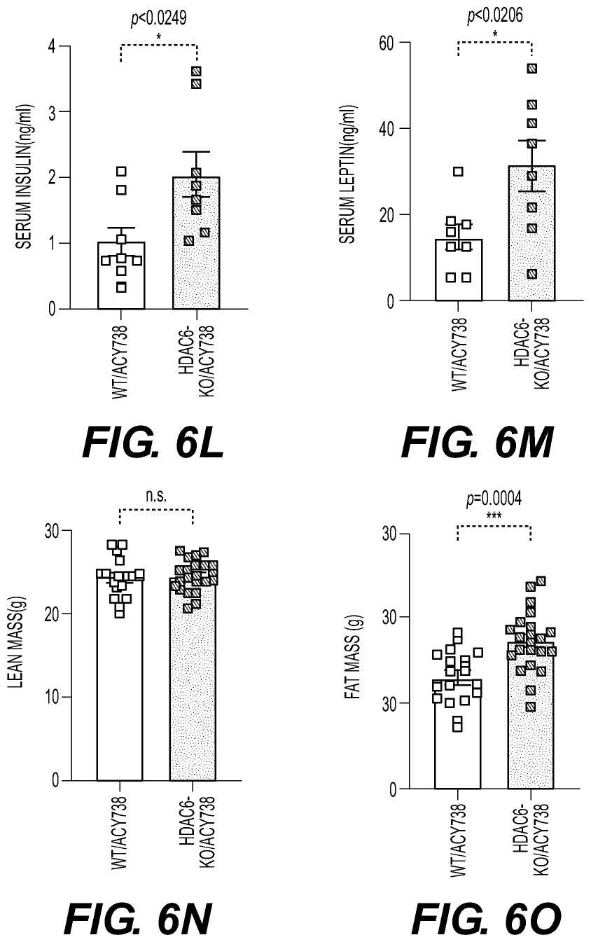

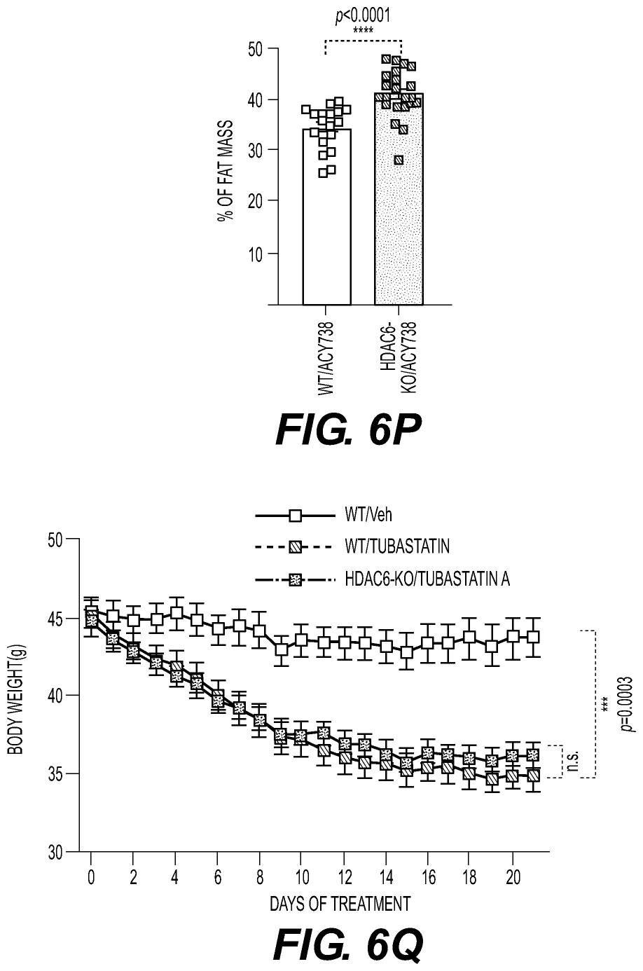

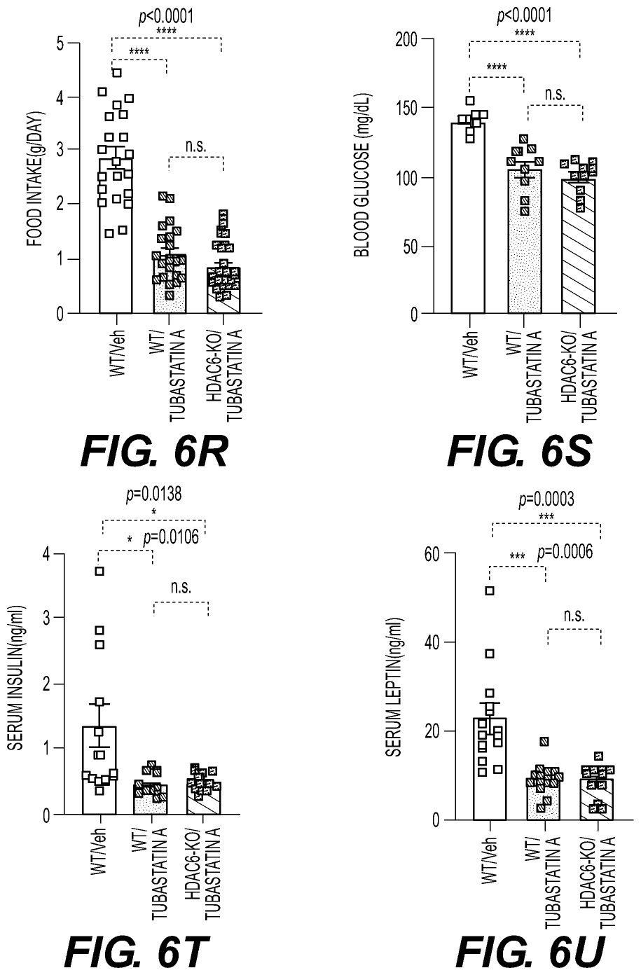

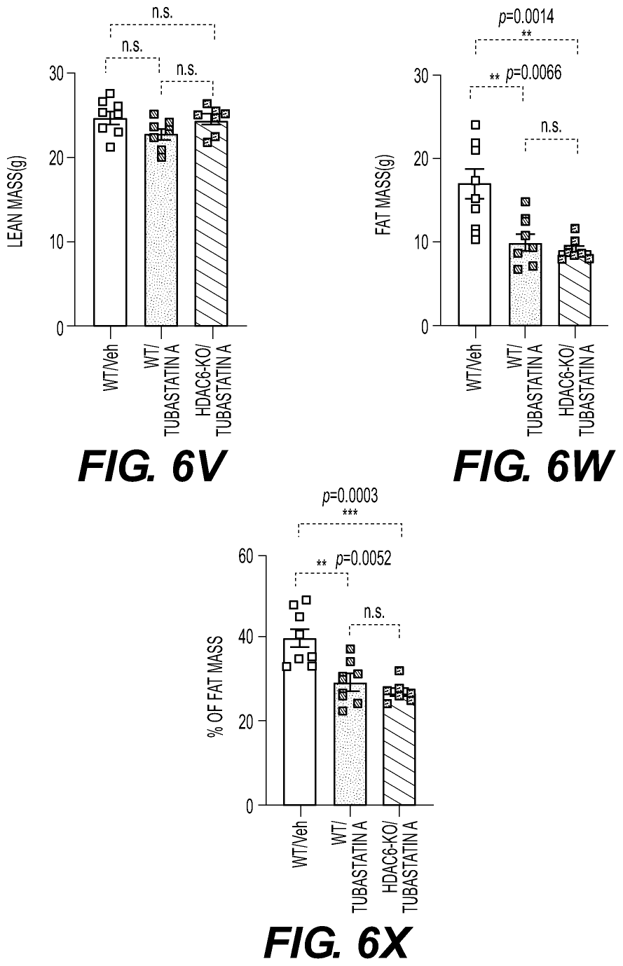

FIG. 6A, Daily body weight of WT (n=20) or HDAC6-KO (n=20) DIO mice during ACY775 (10 mg/kg/day, i.p.) treatment. FIG. 6B, The first week average food intake (g) of WT or HDAC6-KO mice during ACY775 treatment. FIG. 6C, Blood glucose (mg/dl) levels of ACY775 treated WT or HDAC6-KO mice after 1 week of treatment (n=20 for each group). FIG. 6F, Serum insulin (ng/ml) levels of ACY775 treated WT or HDAC6-KO mice after 3 weeks of treatment (n=8 for each group). FIG. 6G, Serum leptin (ng/ml) levels of WT or HDAC6-KO mice after 3 weeks of ACY775 (n=8) treatment. FIG. 6F, lean mass, FIG. 6E, fat mass, FIG. 6H, fat percentage (%) after 3 weeks of ACY775 treatment measured by DEXA scan. FIG. 61, Daily body weight of WT (n=16) or HDAC6-KO (n=20) DIO mice during ACY738 (50 mg/kg/day, i.p) treatment. FIG. 6J, The first week average food intake (g) of WT or HDAC6-KO mice during ACY738 treatment. FIG. 6K, Blood glucose (mg/dl) levels of ACY738 treated WT (n=18) or HDAC6-KO (n=20) mice after one week of treatment. FIG. 6L, Serum insulin (ng/ml) levels of ACY738 treated WT or HDAC6-KO mice after 3 weeks of treatment (n=8 for each group). FIG. 6M, Serum leptin (ng/ml) levels of WT or HDAC6-KO mice after 3 weeks of ACY738 (n=8) treatment. FIG. 6N, Lean mass, FIG. 6O, Fat mass, FIG. 6P, Fat percentage (%) after 3 weeks of ACY738 treatment measured by DEXA scan. FIG. 6Q, Daily body weight of WT or HDAC6-KO DIO mice during the vehicle or Tubastatin A treatment (25 mg/kg/day, i.p, n=15 for WT-Veh, n=17 for WT-Tubastatin A, and n=18 for HDAC6-KO-Tubastatin A). FIG. 6R, The first week average food intake (g) of WT or HDAC6-KO mice during vehicle or Tubastatin A treatment. FIG. 6S, Blood glucose (mg/dl) levels of Veh- or Tubastatin A-treated WT or HDAC6-KO mice after 1 week of treatment (n=8 for WT-veh, n=9 for WT-Tubastatin A, and n=10 for HDAC6-KO-Tubastatin A). FIG. 6T, Serum insulin (ng/ml) levels of vehicle or Tubastatin A treated WT or HDAC6-KO mice after 2 weeks of treatment (n=12 for WT-veh, n=12 for WT-Tubastatin A, and n=13 for HDAC6-KO-Tubastatin A). 6U, Serum leptin (ng/ml) levels of WT or HDAC6-KO mice after 3 weeks of vehicle or Tubastatin A treatment (n=12 for WT-Veh, n=13 for WT-Tubastatin A, and n=14 for HDAC6-KO-Tubastatin A). FIG. 6V, Lean mass, FIG. 6W, Fat mass, FIG. 6X, Fat percentage (%) after 3 weeks of Tubastatin A or vehicle treatment.

HDAC6 knockout mice are resistant to the anti-obesity effect of ACY775 and ACY738, but not of Tubastatin A. Tubastatin A, contrary to specific HDAC6 inhibitors ACY775 and ACY738, improves glucose homeostasis in DIO HDAC6 knock out mice, showing that the mechanism of action is not mediated by inhibition of HDAC6. In contrast, ACY775 and ACY738 inhibit HDAC6 in Agrp neurons to decrease body weight and food intake in DIO mice.

A specific target-based drug should not show its pharmacological effects when the target is depleted or knocked out. Thus, it was decided to test whether the anti-obesity activity of ACY775 and ACY738 is completely mediated through HDAC6. Obesity was induced by feeding a high fat diet for 16 weeks and treating the HDAC6 KO mice first with ACY775 (10 mg/kg, i.p., once a day) for a period of three weeks. ACY775 treatment led to highly significant decrease in the bodyweight of the WT DIO mice when compared to HDAC6 KO DIO mice, indicating that ACY775 anti-obesity effect is truly mediated by HDAC6. The reduction in food intake mediated by ACY775 in the wt group was also blocked in the HDAC6 KO mice. Blood glucose, serum insulin and leptin levels were all significantly higher in the ACY775-treated HDAC6 KO group when compared to the ACY775-treated wt group. DEXA scans revealed that lean mass was not different between the two groups , but fat mass and fat % were significantly lower in the wt group compared to the HDAC6 KO group. Analysis of glucose homeostasis also documented that ACY775 could not increase glucose tolerance and insulin sensitivity in the HDAC6 KO mice when compared to the wt mice treated with ACY775.

Next, the same experiments were performed in the DIO HDAC6 KO and wt mice by administering ACY738 (50 mg/kg i.p., once a day). The results for ACY738 reduced the body weight similar to our previous observations reduced bodyweight of the wt mice around 20%, but also led to a slight decrease in the HDAC6KO mice. However, the difference between HDAC6 KO mice and wt mice treated ACY738 had a major difference and reached to a high significance level (p<0.005). The food intake was significantly higher in the HDAC6 KO group when compared to the wt group. Blood glucose, serum insulin and leptin levels were all significantly higher in the ACY738-treated HDAC6 KO group compared to the wt group. Lean mass was not different between the two groups, but fat mass and fat % were significantly lower in the wt group compared to the HDAC6 KO group. GTT and ITT studies also documented that ACY738 did not improve glucose tolerance and increase insulin sensitivity in the HDAC6 KO mice when compared to the wt mice treated with ACY738. These data indicate that ACY775 and ACY738's anti-obesity effects are mainly mediated by HDAC6 and that HDAC6 KO mouse model is a good tool to dissect the effect of specific HDAC6 inhibitors on obesity.

Tubastatin A reduced the body weight of the HDAC6 KO mice the same as the wt mice. The bodyweight curves of the wt and HDAC6 KO mice treated with Tubastatin A weres almost identical. The food intake was also reduced similarly in the Tubastatin A-treated HDAC6 KO mice when compared to the wt group. Blood glucose, serum insulin and serum leptin levels were all reduced equally in the wt and HDAC6 KO group. Similarly, lean mass was not different between the three groups, but fat mass and fat % were significantly lower in both wt and HDAC6 KO groups treated with Tubastatin A.

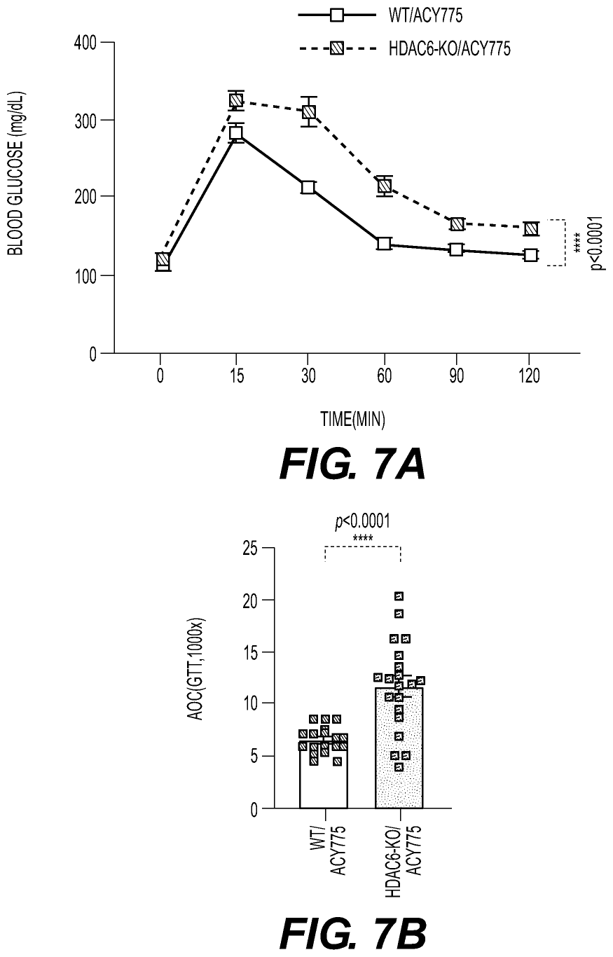

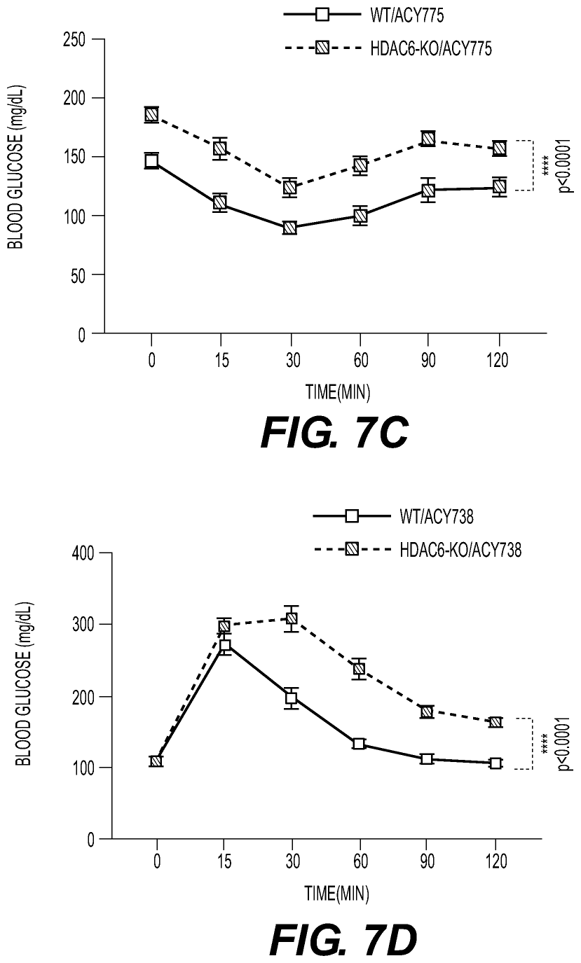

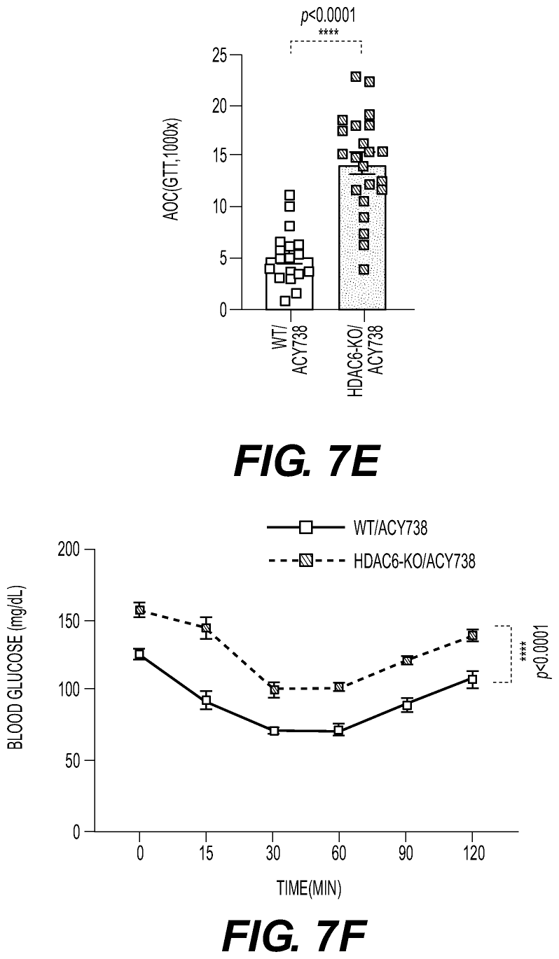

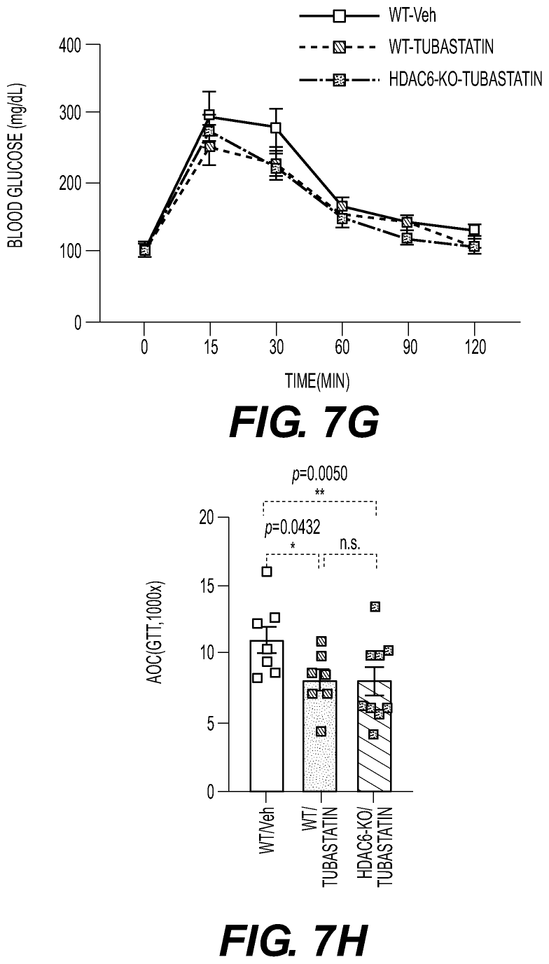

The effects of ACY 775, ACY738 and tubastatin in GTT and ITT studies (FIG. 7A-7C, ACY 775; FIG. 7D-7F, ACY738, and FIG. 7G-7I, tubastatin) showed that Tubastatin A improved glucose tolerance and increased insulin sensitivity in both wt and the HDAC6 KO mice when compared to the wt mice treated with Veh. These data indicate that Tubastatin A's anti-obesity effects are not mediated by HDAC6, but mediated either through off target effects or through induction of toxicity.

### Example 8: A Poor Blood Brain Barrier Permeable Specific HDAC6 Inhibitor Ricolinostat Neither Increases Leptin Sensitivity Nor Reduces Body Weight

### Materials and Methods

DIO mice were pretreated with ricolinostat (25 mg/kg/day, i.p.) or vehicle for 3 days, which was followed by co-treatment with ricolinostat (or vehicle) and leptin (1 mg/kg, i.p.) (or saline) (n=32 for each group). Change in body weight 15 h after leptin treatment, and food intake (g) during the 15 h period after leptin treatment were determined. The results were reproduced in two independent experiments.

DIO mice were pretreated with ricolinostat (50 mg/kg/day, i.p.) or vehicle 3 days, which was followed by a last injection of ricolinostat (or vehicle) in the morning of fourth day. 6 hours later mice were injected with saline or leptin (1 mg/kg, i.p.) and the hypothalamus were extracted after 45 minutes following the leptin injection. Immunoblots were performed for STAT3^{Tyr705} phosphorylation and total STAT3 from the hypothalamus. The ratio of p-STAT3^{Tyr705} to total STAT3 signal in the immunoblots was quantified.

DIO mice then were treated with ricolinostat (50 mg/kg, i.p.) for 3 weeks. Daily body weight (g), daily food intake (g), serum leptin (ng/ml) levels after 3 weeks of vehicle (n=8) or ricolinostat (n=8) treatment, lean mass (g); fat mass and fat percentage (%) after 3 weeks of vehicle (n=16) or ricolinostat (n=17) treatment measured by DEXA scan were determined.

Blood glucose (mg/dl) levels were measured after 1 week of vehicle (n=16) or ricolinostat (n=16) treated DIO mice. GTT after 1 week of vehicle (n=17) or ricolinostat (n=16) treatment of DIO mice was assessed. AOC analysis for GTT was performed. ITT after 2 weeks of vehicle (n=17) or ricolinostat (n=16) treated DIO mice was measured. Serum insulin (ng/ml) levels after 3 weeks of vehicle (n=8) or ricolinostat (n=8) treatment were measured in DIO mice. Liver sections from 3 weeks of vehicle or ricolinostat treated DIO mice were stained by H&E.

### Results

HDAC6 directly interacts with LepRb and reduces its activity. HDAC6 inhibitor ACY775 blocks this interaction. LepRb is expressed in the central nervous system and leptin's anorexigenic and weight reducing effect is mainly mediated by this isoform of LepR.

These results in Example 7 show that Tubastatin anti-obesity effect is not mediated by HDAC6. The in vitro and in vivo data suggested a possible central effect of HDAC6. If this hypothesis is correct, then a highly specific HDAC6 inhibitor with low blood brain barrier (BBB) permeability would be ineffective in increasing leptin sensitivity and reducing the bodyweight and food intake of the DIO obese mice. One such agent is Ricolinostat. ACY775 has a brain/plasma ratio of about 1.26, ACY738 has a brain/plasma ratio of approximately 1.22 but Ricolinostat has brain plasma ratio of 0.01, which has a poor BBB permeability.

Ricolinstat's ability to increase tubulin acetylation in the cells was tested to confirm its HDAC6 inhibitor activity. After confirmation of Ricolinstat's HDAC6 inhibitory activity, it was investigated whether Ricolinostat increases leptin sensitivity of the DIO mice. The same experimental protocol for the ACY738 and ACY775 was used.

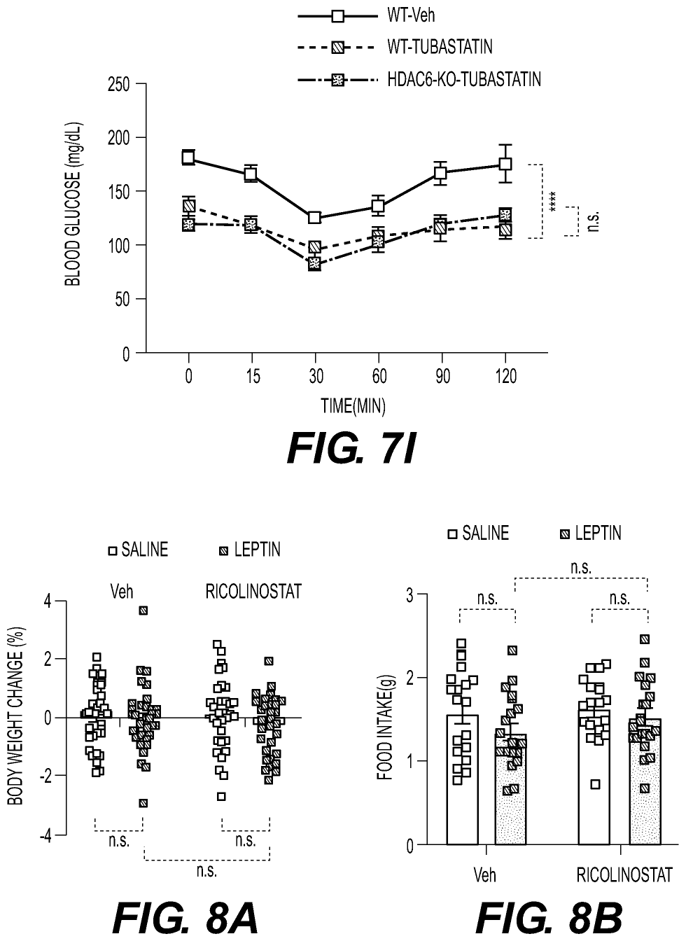

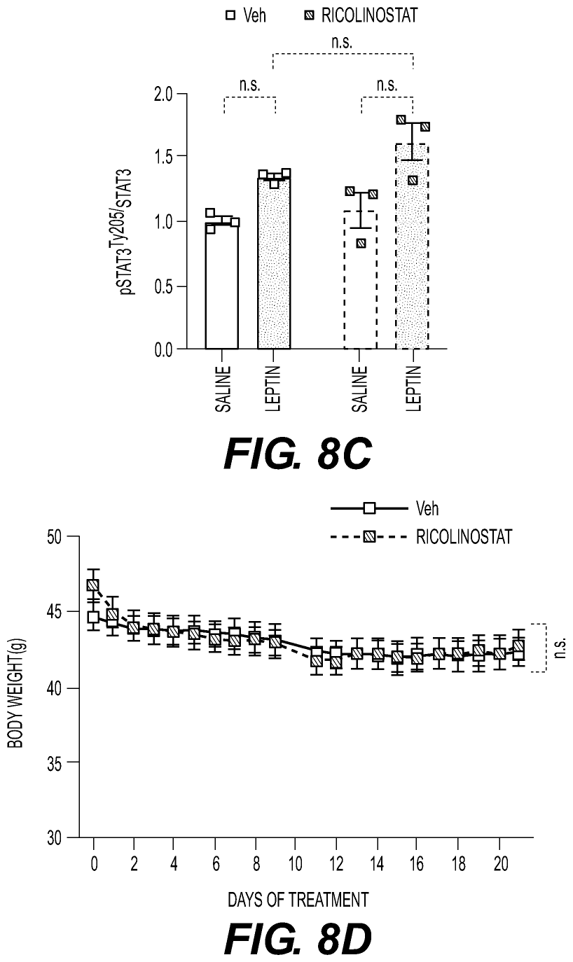

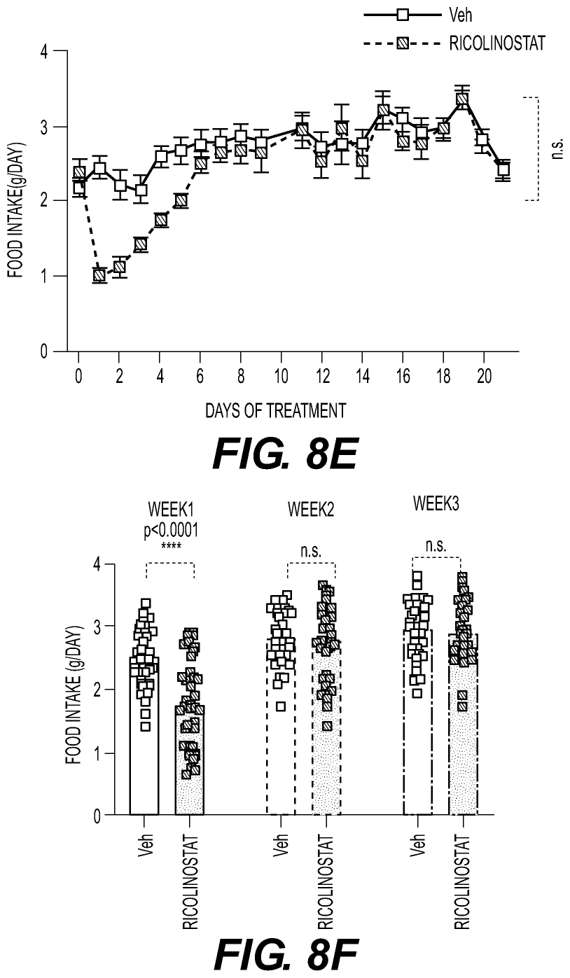

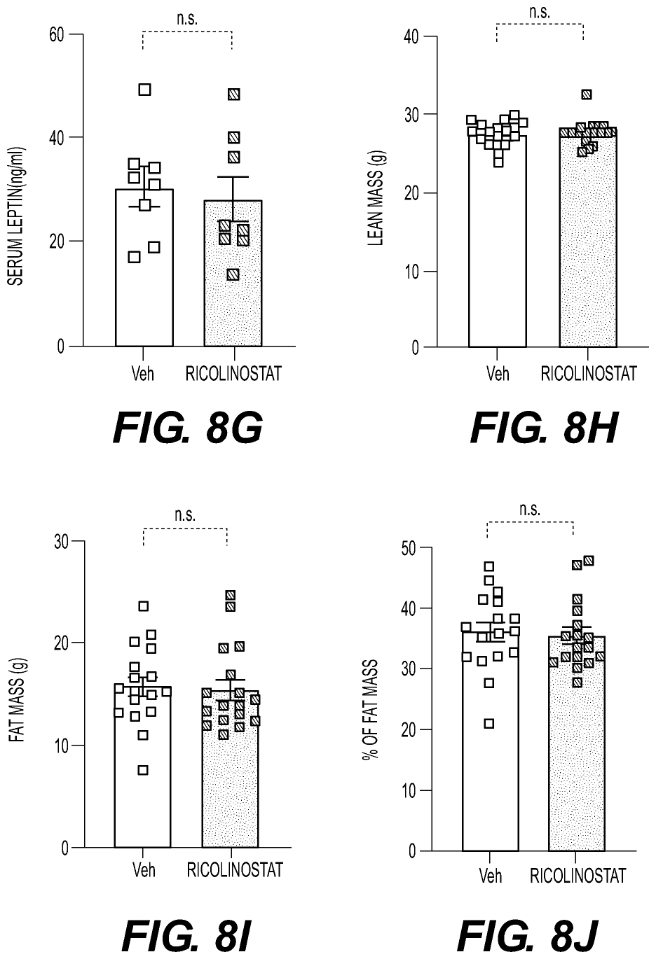

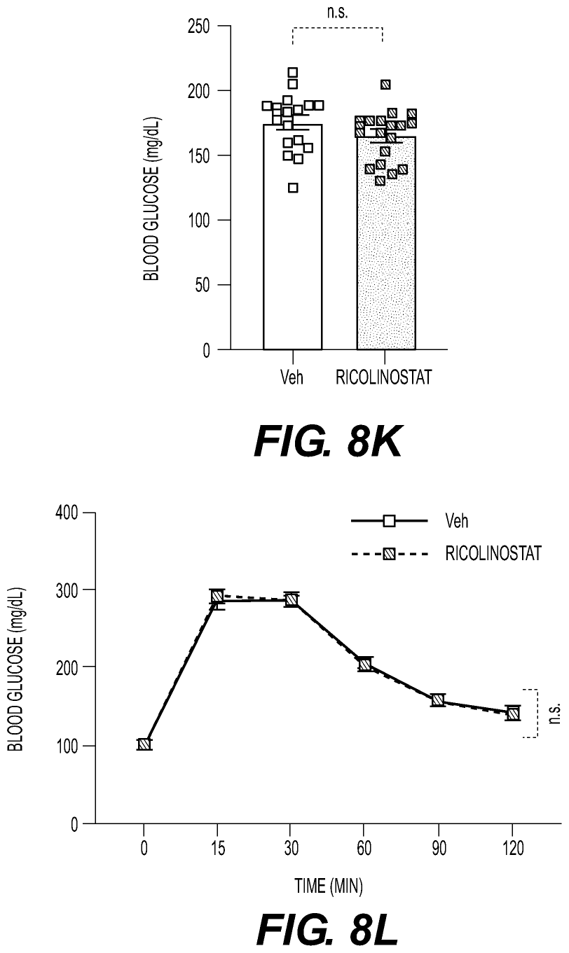

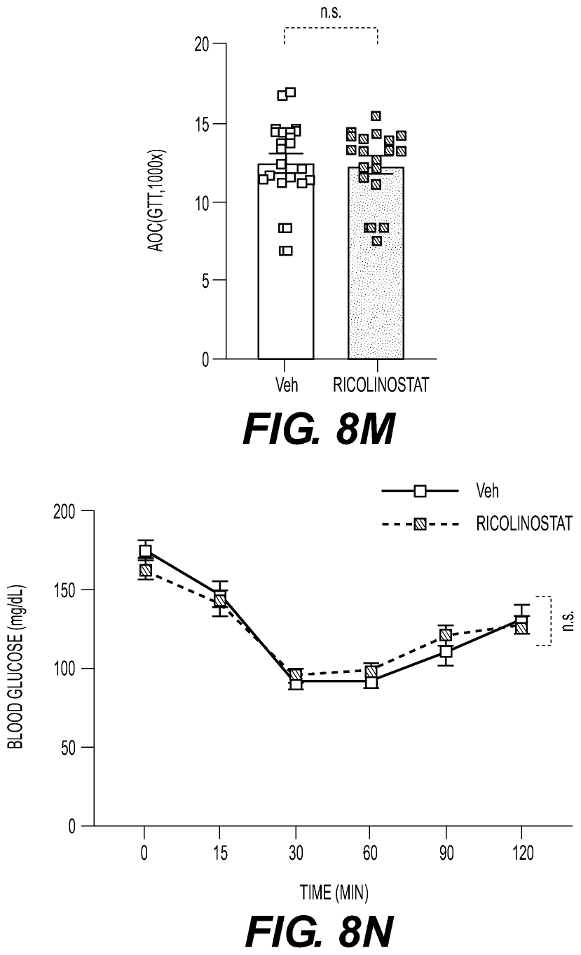

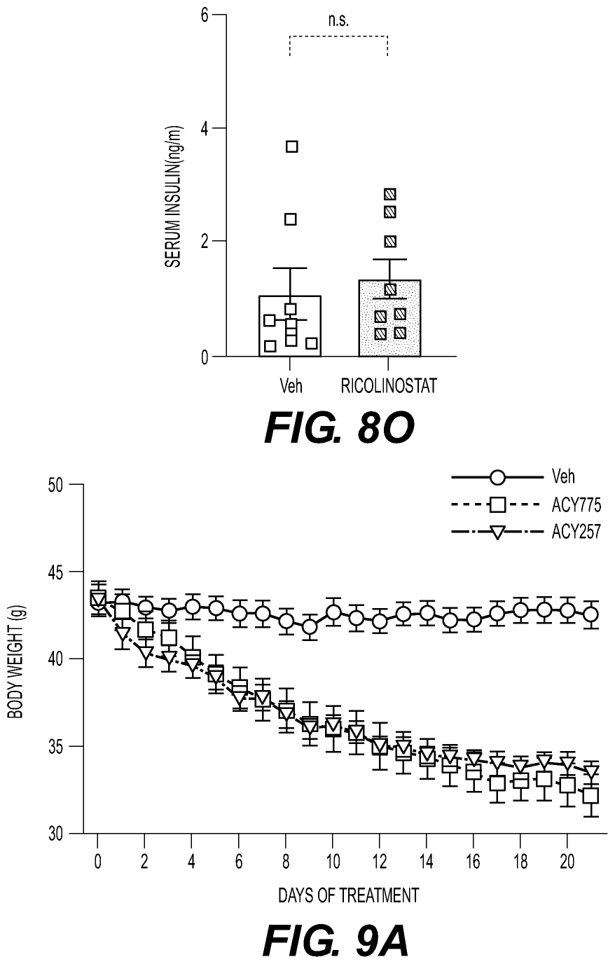

The results shown in FIGS. 8A-80 demonstrate that a poor blood brain barrier permeable HDAC6 inhibitor, Ricolinostat, is not a leptin sensitizer and anti-obesity agent.

Administration of leptin to DIO mice did not change their bodyweight, compared to the control group (FIG. 8A). Ricolinostat treatment (25 mg/kg, i.p.) alone also did not decrease the bodyweight of DIO mice, and administration of leptin to DIO mice that were pre-treated with Ricolinostat also did not reduce the body weight. Ricolinostat treatment alone or together with leptin had no effect on the food intake of DIO mice (FIG. 8B). Investigation of the in vivo activation of leptin receptor signaling following Ricolinostat treatment revealed that leptin injection to Veh- or Ricolinostat-treated DIO mice did not significantly increase STAT3^{Tyr705} phosphorylation (FIG. 8C). These results show that acute treatment of DIO mice with Ricolinostat, unlike ACY738 and ACY775, does not increase leptin sensitivity. These results were consistent with the results of DIO mice treated with ricolinostat (50 mg/kg, i.p.) for 3 weeks as shown by FIGS. 8D-8O.

Ricolinostat was chronically administered to three different cohorts. The first cohort received (25 mg/kg, i.p., once a day) and the 2^{nd} and 3^{rd} cohorts received 50 mg/kg once a day administration of Ricolinostat for period of 3 weeks. Administration of Ricolinostat (50 mg/kg, i.p., once a day administration) led to a minimal decrease in bodyweight for the first couple of days without creating any meaningful or significant decreases during the 3-week treatment (FIG. 8D). Food intake was reduced transitionally during the first 2-3 days of treatment but then rebounded and did not change during the course of treatment (FIGS. 8E, 8F). In line with unaltered bodyweight or food intake during the chronic Ricolinostat treatment, serum leptin levels (FIG. 8G), lean mass (FIG. 8H), fat mass (FIG. 8I), percent fat (FIG. 8J)were not different between the Veh- and Ricolinostat-treated mice).

Ricolinostat treatment did not reduce blood glucose levels (FIG. 8L), did not improves glucose tolerance (FIG. 8M) or insulin sensitivity (FIG. 8N) and did not reduce insulin levels (FIG. 80). Furthermore, there were no difference in the H&E staining of the liver and no decrease in the hepatic steatosis.

Thus, the results show Ricolinostat does not reduce body weight, thereby supporting the premise that HDAC6 inhibitors must penetrate into the hypothalamus to be effective to weight and glucose control.

### Example 9: ACY257 is as Effective as ACY775

### Materials and Methods

Studies were conducted to compare efficacy in reducing body weight in another HDAC6 inhibitor, ACY257, with that of ACY775.

Studies were conducted as described above with reference to ACY775.

### Results

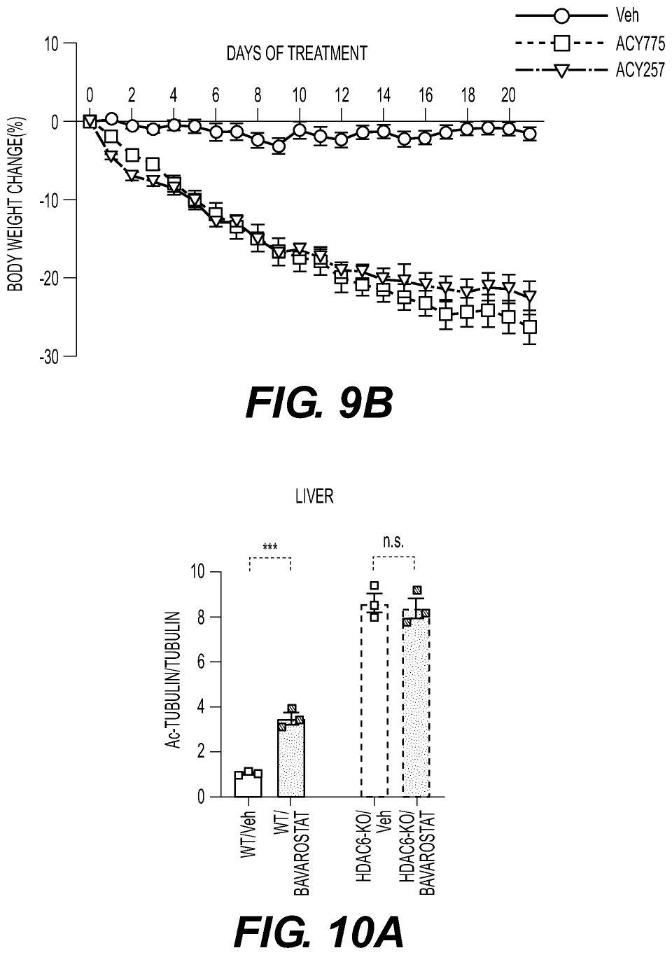

FIGS. 9A and 9B are comparison of body weight over time (2P) and % change in body weight over time, comparing ACY775 and ACY257 to vehicle.

The results demonstrate that ACY257 is as effective as ACY775 in reducing body weight, and provides further evidence that only HDAC6 inhibitors which penetrate the hypothalamus and inhibit HDAC6 therein are effective to treat obesity, especially leptin-resistant obesity. Data demonstrates that ACY 257, which inhibits HDAC6 in the hypothalamus but does not inhibit HDAC6 in other brain regions such as cerebrum, frontal lobe, temporal lobe, and brainstem, greatly reduces body weight. The hypothalmus is a very small part of the brain. The hypothalamus has a slightly more permeable blood brain barrier compared to other parts of the brain. The data indicates that inhibitors of HDAC6 which have the ability to preferentially pass through the BBB and into the hypothalamic region, especially the arcuate nucleus, are able to reduce body weight.

### Example 10: HDAC6 in AgRP Expressing Neurons are Direct Target and Mediator of HDAC6 Inhibitors and DMH Neurons are Required for HDAC6 Inhibitor's Appetite and Bodyweight Reducing Effect in DIO Mice

### Materials and Methods

HEK293 cells were transfected with plasmids expressing mouse HDAC6 cDNA with myc tag at c-terminal and Cas9, infected with AAV-sgHDAC6, and then harvested for western blotting with antibodies against myc or Tubulin.

Design, validation, and stereotaxic injection of AAV viral vector carrying the guide RNAs targeting the mouse HDAC6 genomic locus. Three designed sgRNAs were concatenated and constructed into an AAV vector carrying a cre-enabled mCherry fluorescent marker.

Following the bilateral injection of AAV-sgHDAC6 into the arcuate nucleus of Agrp-ires-cre::LSL-Cas9-GFP (Agrp-Cre^{+/-}::Cas9^{+/-}) or control LSL-Cas9-GFP (Cas9^{+/-}) mice, immunostaining to detect the expression of mCherry and GFP was performed. AAV-sg-HDAC6 injected Agrp-Cre^{+/-}::Cas9^{+/-} (HDAC6^{Agrp-Δ}) and Cas9^{+/-} (HDAC6^{WT}) DIO mice (n=7 for each group) were fasted for 24 hours, and injected with ACY775 (10 mg/kg. i.p). Hourly total dark cycle food intake (g) (n=7 for each group) of these mice after ACY775 injection was measured. Body weight of AAV-sg-HDAC6 injected HDAC6^{Agrp-Δ} (n=11) and HDAC6^{WT} (n=6) mice were measured during 3 weeks of ACY775 (10 mg/kg/day, i.p.) treatment. Daily average food intake of HDAC6^{Agrp-Δ} (n=11) and HDAC6^{WT} (n=6) mice was measured during 3 weeks of ACY775 (10 mg/kg/day, i.p.) treatment. 24-hour food intake average of HDAC6^{Agrp-Δ} and HDAC6^{WT} mice during the 3 weeks of ACY775 treatment was assessed. Serum leptin level (ng/ml) of HDAC6^{Agrp-Δ} and HDAC6^{WT} mice after 2 weeks of ACY775 treatment (n=13 for HDAC6^{WT} and n=18 for HDAC6^{Agrp-Δ}) was measured. Lean mass (g); fat mass; and fat percentage (%) of HDAC6^{Agrp-Δ} and HDAC6^{WT} DIO mice after 3 weeks of ACY775 treatment was measured by DEXA scan. The HDAC6 inhibitor responsive neurons in the dorsomedial hypothalamus (DMH) were assessed. Immunostaining and quantification of c-Fos expression was performed in the DMH of DIO mice following one-time ACY775 treatment (10mg/kg, i.p.).

Coordinates used for aav-sg-HDAC6 injection into the DMH of c-Fos-iCreER::LSL-Cas9-GFP(TRAP2-Cas9) mouse., mCherry immunostaining in the aav-sg-HDAC6 injected TRAP2-cas9 obese mice, which were treated three weeks earlier with one dose of ACY775 (10 mg/kg, i.p.) (TRAP2-HDAC6^{DMH-Δ}) or vehicle (TRAP2-HDAC6^{WT}), followed by 4-Hydroxytamoxifen (4-OHT) injection, were assessed. TRAP2-HDAC6^{WT} and TRAP2-HDAC6^{DMH-Δ} mice were fasted 24-h and treated with ACY775 (10 mg/kg, i.p.). Hourly and total dark cycle food intake (g) (n=4 for each group) was determined for these mice after ACY775 injection. Daily body weight of TRAP2-HDAC6^{DMH-Δ} (n=10) or TRAP2-HDAC6^{WT} (n=11) DIO mice was measured during 2 weeks of ACY775 (10 mg/kg/day, i.p) treatment. Daily average food intake of TRAP2-HDAC6^{DMH-Δ} (n=10) or TRAP2-HDAC6^{WT} (n=11) DIO mice was measured during 2 weeks of ACY775 treatment.

### Results

The results demonstrated that HDAC6 in AgRP expressing neurons are the direct target and mediator of HDAC6 inhibitors that are effective for weight and glucose control, including ACY775, ACY738 and ACY 257. DMH neurons are required for HDAC6 inhibitor's appetite and bodyweight reducing effect in DIO mice

The AgRP-expressing neurons in the ARC of the hypothalamus (AgRP^{ARC} neurons) have been shown to be one of the major neuronal populations in the brain mediating leptin's effects on feeding, bodyweight and energy balance. Considering that fasting increases the expression of HDAC6 in the AgRP^{ARC} neurons and that HDAC6 binds to LepRb and reduce its activity, and taking into account the inability of low BBB permeable HDAC6 inhibitors in increasing leptin sensitivity and reducing bodyweight, it was hypothesized that HDAC6 in AgRP^{ARC} neurons mediates the effects of HDAC6 inhibitors on appetite and body weight. To investigate this, 3 single-guide RNAs (sgRNAs) were designed in tandem to target three different exons of the mouse HDAC6 locus and their efficacy in vitro in cultured HEK293 cells validated. The three sgRNAs were constructed into an adeno-assisted viral vector (AAV-sgHDAC6) with a cre-dependent mCherry reporter to indicate virus-transduced neurons. To assess the functional relevance of HDAC6 in AgRP^{ARC} neurons, AAV-sgHDAC6 was bilaterally injected into the ARC of LSL-Cas9-GFP and Agrp-IRES-cre::LSL-Cas9-GFP DIO mice and observed extensive colocalization of cre-enabled mCherry with GFP immunosignal in the ARC of Agrp-IRES-cre::LSL-Cas9-GFP (HDAC6^{Agrp-Δ}) mice, but not in LSL-Cas9-GFP (HDAC6^{WT}) control mice.

The HDAC6^{WT} and HDAC6^{Agrp-Δ} DIO mice were treated with a single dose of (ACY775, 10 mg/kg, i.p.). It was found that HDAC6^{Agrp-Δ} DIO mice exhibited almost complete resistance to ACY755-induced suppression of food intake. These results prompted performing chronic ACY775 treatment to see whether all the anti-obesity effects of ACY775 is mediated through HDAC6 in AgRP neurons. HDAC6^{Agrp-Δ} and HDAC6^{WT} DIO mice were treated with ACY775 (10 mg/kg/day, i.p.) for three weeks.

Significantly attenuated effects of ACY775 on all the parameters were observed, including weight loss, long-term appetite, leptin levels, adiposity, and glucose homoestasis. These data strongly indicate that HDAC6 in AgRP^{ARC} neurons, not in a peripheral tissue, are the primary target of the HDAC6 inhibitors for their anti-obesity action. Similarly, deletion of HDAC6 in AgRP^{ARC} neurons did not change Tubastatin A's effects on suppressing feeding or body weight.

To investigate whether activity of other neuronal populations are altered after HDAC6 inhibitor treatment, c-fos staining was performed in the whole brain of DIO mice, which were acutely treated with ACY775. There was a major increase in c-fos positive cell number in the dorsomedial hypothalamic nucleus (DMH) of ACY775-treated DIO mice at fasting conditions. Some of the DMH neurons express high levels of LepRb and involved in regulation of appetite (ref). Whether HDAC6 in these ACY775-responsive DMH neurons is required or involved in any way in mediating the appetite suppressing and body weight lowering effects of ACY775 was then tested. To deplete HDAC6 in these ACY775-responsive DMH neurons, c-fos-icreER (TRAP2) were crossed with LSL-cas9-GFP lines to generate c-fos-icreER:: LSL-cas9-GFP (TRAP2-cas9) mice and induced obesity by 20 weeks of HFD feeding. Subsequently, aav-sgHDAC6 were injected bilaterally into the DMH of TRAP2-cas9 DIO mice followed by a single dose of ACY775 (or Vehicle, as control) and 4-hydroxytamoxifen (4-OHT) injection to specifically allow sgHDAC6 to deplete HDAC6 only in ACY775 induced- and c-fos cre-activated DMH neurons)) (TRAP2-HDAC6^{DMH-Δ}.

Three weeks later 24-hours fasted TRAP2-HDAC6^{DMH-Δ} and TRAP2-HDAC6^{WT} DIO mice were treated with a second dose of ACY775, and it was found that mice from both groups consumed similar but small amount of food. Furthermore, mice in both TRAP2-HDAC6^{DMH-Δ} and TRAP2-HDAC6^{WT} groups lost weight and food intake was reduced equally in both groups during the two weeks of ACY775 treatment. These results indicate that HDAC6 in DMH responsive neurons is not required for HDAC6 inhibitor's appetite and bodyweight reducing effects.

Whether the activity of ACY775-responsive DMH neurons could be involved in the food intake and bodyweight reducing activity of HDAC6 inhibitors was investigated as a possible second order of neurons to AgRP^{ARC} neurons. These neurons were ablated to investigate the necessity of these neurons in mediating the ACY775's anti-obesity effects. AAV-flex-taCasp3-TEVp was injected into the DMH of TRAP2 DIO mice,nd then the mice were injected with a single dose of ACY775 and 4-OHT to ablate these neurons through caspase3 protein activation. To make sure that this experimental system worked, the existence of ACY775-responsive DMH neurons was verified by c-fos staining after injection of a second dose of ACY775. This experimental method predicts that caspase 3, when activated by administration of the first dose of ACY775 in conjugation with 4-OHT injection, will lead to ablation of ACY775-responsive DMH neurons, and diminish the c-fos signals that would otherwise be created by the second dose of ACY775.

The results documented that the ACY775-stimulated increase in c-fos positive cells in DMH almost completely disappeared in the ACY775 and 4-OHT injected TRAP2 DIO mice (caspase3^{on-DMH}). However, c-fos positive DMH neurons after ACY755 treatment were still detectable in the Vehicle- and 4-OHT-injected TRAP2 DIO mice (caspase3^{off-DMH}). These results confirmed that ACY755-responsive DMH neurons were successfully ablated in the caspase3^{on-DMH} mice. After confirming the ablation, the fasted caspase3^{on-DMH} and caspase3^{off-DMH} mice were treated with ACY775 (10 mg/kg, i.p.) and it was documented that the food intake either measured hourly or during the whole dark cycle in caspase3^{on-DMH} mice was significantly higher than caspase3^{off-DMH} mice.

To exclude the possibility that caspase3^{on-DMH} mice might have problems in general for hunger sensing, both caspase3^{on-DMH} and caspase3^{off-DMH} mice were refed after overnight fasting, and it was confirmed that all groups of mice consumed similar levels of food. Both caspase3^{on-DMH} and caspase3^{off-DMH} mice were then chronically treated with ACY775 (10 mg/kg/day, i.p.) and it was found that ACY775 treatment neither reduced the body weight nor the food consumption in caspase3^{on-DMH} mice, in which the ACY775-responsive DMH neurons were ablated. ACY775 treatment did reduce the food intake and bodyweight in caspase3^{on-DMH} mice. Consistently, blood glucose levels were significantly higher in caspase3^{on-DMH} mice when compared to the caspase3^{off-DMH} DMH mice. These data indicate that the ACY775-responsive DMH neurons are necessary to mediate ACY775's appetite and bodyweight reducing effect in DIO mice.

Next, the ACY775-responsive DMH neurons were chemogenically activated to investigate whether activation of these neurons would be sufficient to reduce the food intake and the bodyweight. Adeno-associated virus (AAV) carrying Cre-dependent hM3Dq-DREADD fused to mCherry transgene (AAV-DIO-hM3Dq-mCherry) was injected bilaterally into the DMH of TRAP2 DIO mice. Four weeks after injections, a single dose of ACY775 (10 mg/kg, i.p.) or vehicle was injected to induce c-fos expression. Subsequently, 4-OHT injection was performed after 5 h to activate c-fos driven Cre in the fasted DIO mice. The mCherry signal was expressed only in ACY775-treated TRAP2 DIO mice and was absent in vehicle-treated TRAP2 DIO mice.

Next, both AAV-DIO-hM3Dq-mCherry- and ACY775-injected, and vehicle-treated TRAP2 DIO mice with saline were treated for 3 days and with clozapine N-oxide (CNO) for 4 days. Bodyweight and food intake of all groups during saline treatment period were similar and did not show any significant differences between the groups. However, the mice from ACY775-induced group showed significant reduction in bodyweight and food intake during the CNO treatment while Veh-induced TRAP2 DIO mice did not. The blood glucose levels at the end of saline and CNO treatment was also measured. It was demonstrated that CNO treatment significantly decreased blood glucose levels only in AAV-DIO-hM3Dq-mCherry injected and ACY775-induced TRAP2 DIO mice. Blood glucose lowering effect of CNO was absent compared in Veh-induced TRAP2 DIO mice and in all the saline-treated mice. Taken together, the data show that the ACY775-responsive DMH neurons are both necessary and also sufficient to mediate ACY775's appetite and bodyweight reducing effect in DIO mice.

### Example 11: Bavarostat as an HDAC6 Inhibitor in the Hypothalamus

### Materials and Methods

Bavarostat (4-(((((3r,5r,7r)-adamantan-1-yl)methyl)(methyl)amino)methyl)-3-fluoro-N-hydroxybenzamide) is an HDAC6-selective inhibitor with high brain penetrance, Strebl, et al. ACS Cent Sci. 2017 Sep 27; 3(9): 1006-1014.

DIO mice were intraperitoneally injected with bavarostat (10mg/kg). After 30 minutes, protein from liver and hypothalamus tissues were subjected to Ac-tubulin immunoblotting to investigate HDAC6 inhibitor activity in the liver and hypothalamus.

Immunoblotting for Ac-tubulin in the liver and hypothalamus tissues from bavarostat treated DIO mice was performed. The ratio of Ac-tubulin to total tubulin signal in the immunoblots of liver and brain was quantified.

DIO mice then were treated with bavarostat (10 mg/kg, i.p.) for 3 weeks and daily body weight (g) of DIO mice treated with Veh (n=9) or bavarostat (n=9) measured during the three weeks of treatment period. Daily food intake (g) during 3 weeks of Veh or bavarostat treatment was also measured.

### Results

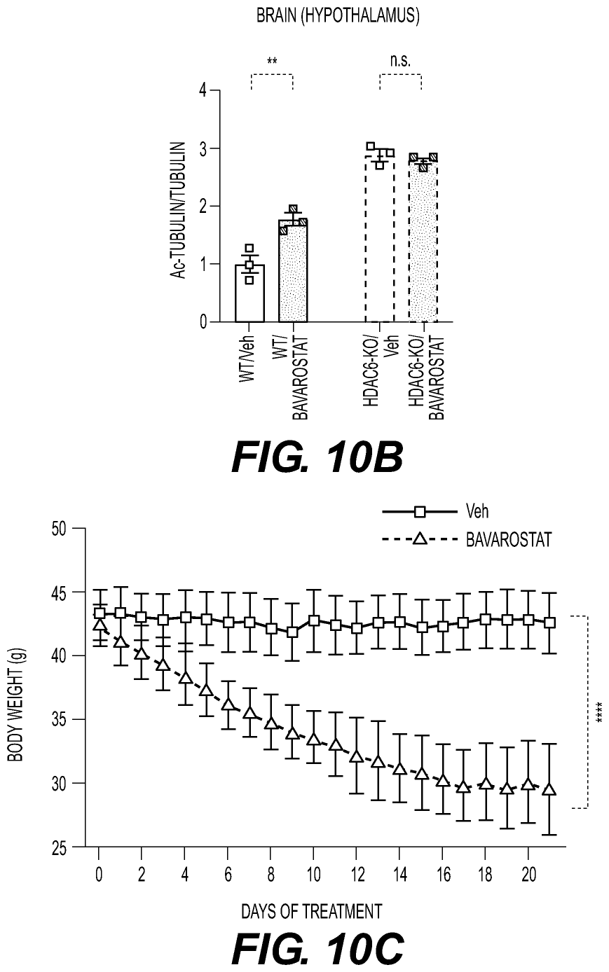

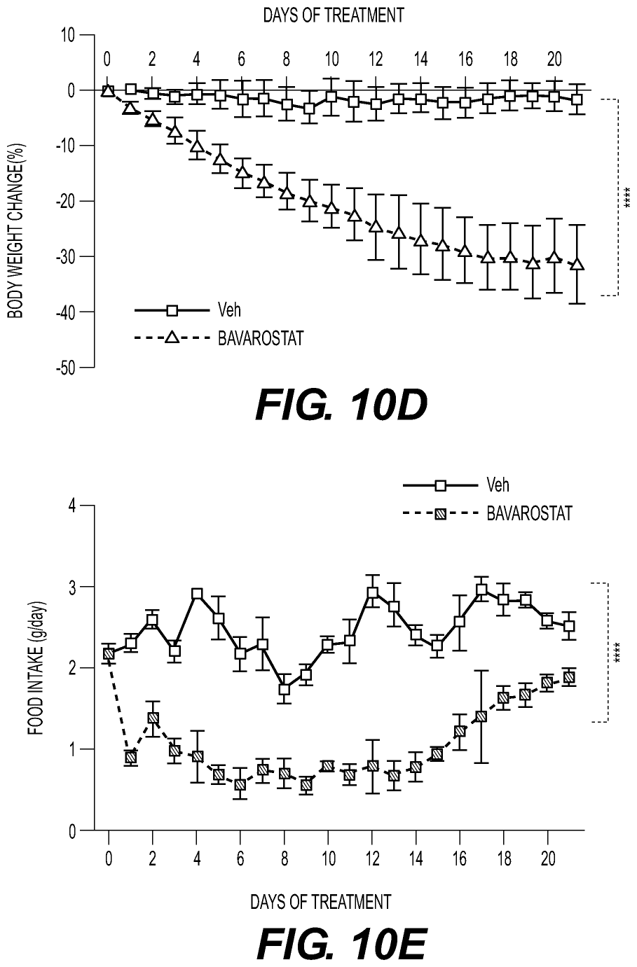

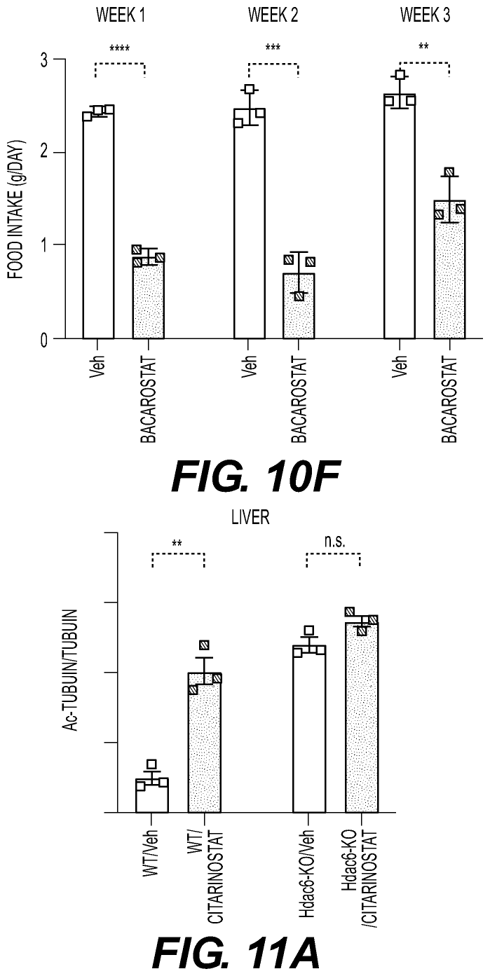

FIG. 10A-10F show that treatment of wild type mice but not HDAC6 Knockout animals with bavarostat caused weight loss in obese animals. Appetite was initially suppressed but then rose over time.

### Example 12: Citarinostat is Not Effective as an HDAC6 Inhibitor in the Hypothalamus

Citarinostat is a tetrahydroquinoline-based selective histone deacetylase 6 (HDAC6) inhibitor, with known pharmacological and ADMET properties, and ability to improve upon memory performance in a mouse model of FXS, Fmr1−/− mice. This small molecule demonstrates good brain penetrance, low-nanomolar potency for the inhibition of HDAC6 (IC50=2.3 nM), with at least a thousand-fold selectivity over all other class I, II, and IV HDAC isoforms, through its inhibition of the α-tubulin deacetylase domain of HDAC6 (CD2) Kozikowski, et al. ACS Chem Neurosci. 2019 Mar 20; 10(3): 1679-1695. Citarinostat (ACY-241, HDAC-IN-2) is an orally available selective HDAC6 inhibitor with IC50 of 2.6 nM and 46 nM for HDAC6 and HDAC3, respectively. It has 13 to 18-fold selectivity towards HDAC6 in comparison to HDAC1-3.

### Materials and Methods

DIO mice were intraperitoneally injected with citarinostat (25mg/kg) for 30 minutes. Protein from liver and brain (hypothalamus) tissues were subjected to Ac-tubulin immunoblotting to investigate HDAC6 inhibitor activity in the liver and brain (hypothalamus). a, Immunoblotting of Ac-tubulin in the liver and brain (hypothalamus) tissues from citarinostat treated DIO mice. Quantification of the ratio of Ac-tubulin to total tubulin signal in the immunoblots of liver and brain in DIO mice treated with citarinostat (25 mg/kg, i.p.) for 3 weeks, was assessed. Daily body weight (g) of DIO mice treated with Veh (n=12) or citarinostat (n=11) was also measured during 3 weeks of treatment period. Daily body weight change (%) of DIO mice during 3 weeks of treatment period, as well as daily food intake (g) and weekly average 24 h food intake during the Veh or citarinostat treatment.

### Results

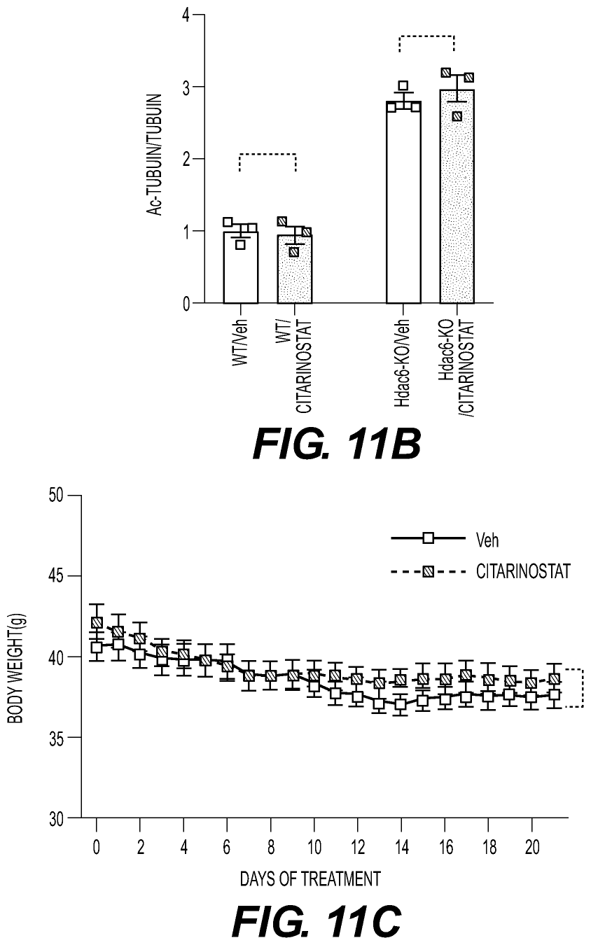

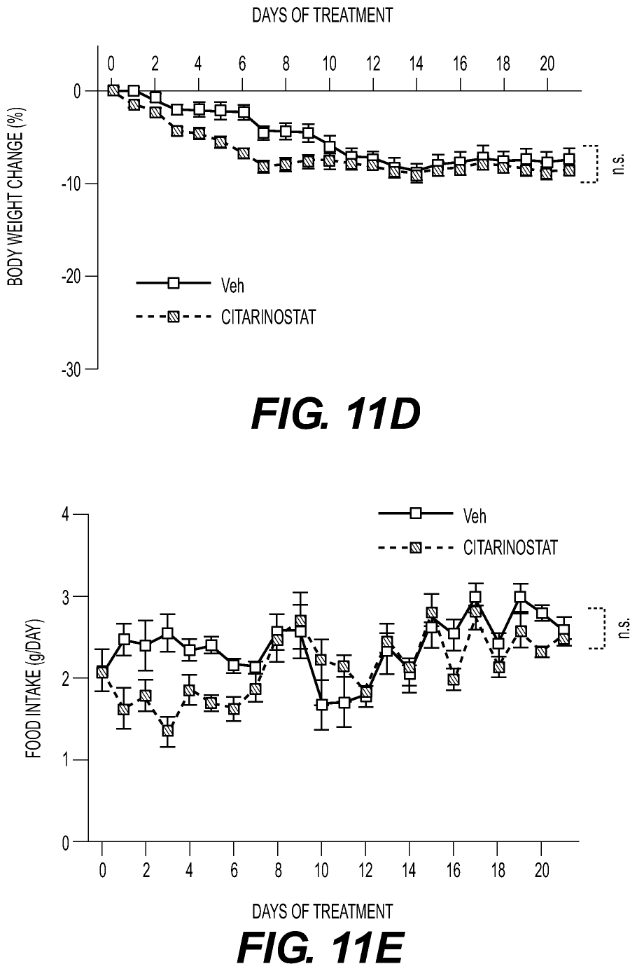

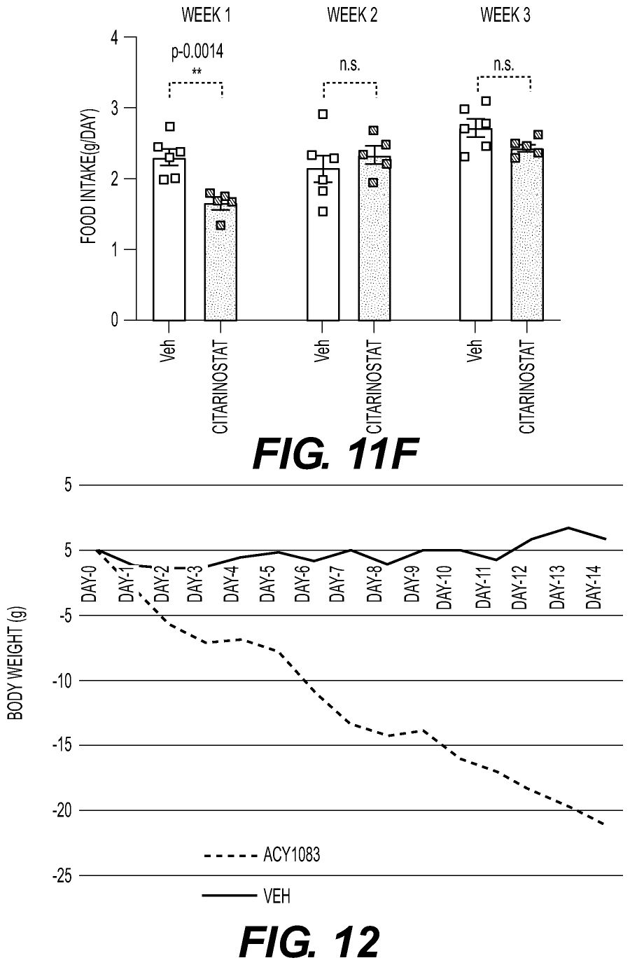

The results shown in FIGS. 11A-11F demonstrate that it is only HDAC6 inhibitors what are inhibitory in the hypothalamus that are useful for obesity treatment. Citarinostat inhibits HDAC6 in other brain regions such as cerebrum, frontal lobe, temporal lobe, brainstem but not in the hypothalamus, Therefore it is not effective in reducing body weight.

### Example 13: Administration of ACY1083 is Effective in Restoring Leptin Sensitivity as Assessed by Weight Loss

### Materials and Methods

ACY1083 was administered to DIO mice as described in Example 3.

### Results

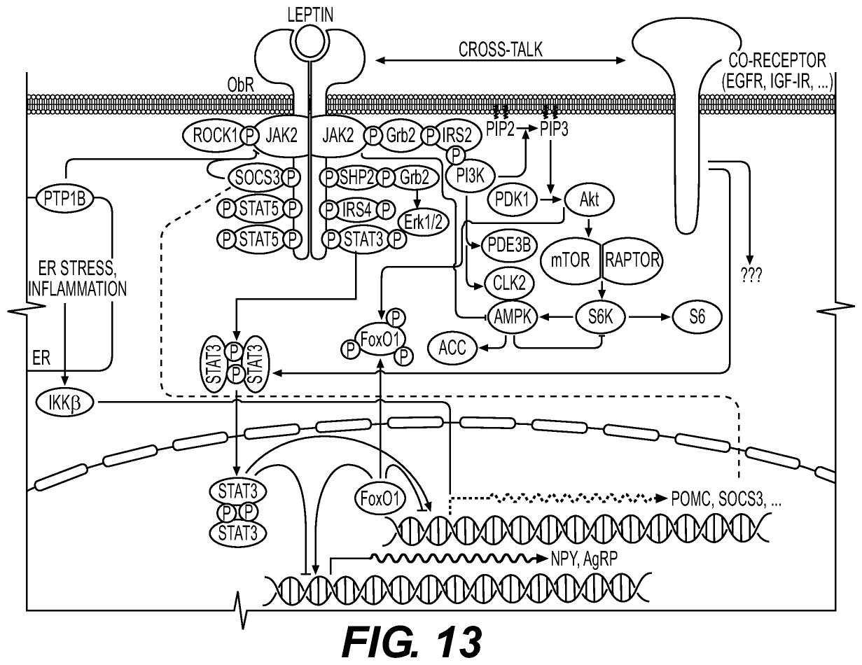

The results are shown in FIG. 12. The ACY1083 caused significant decrease in body weight over 14 days.

### Summary and Conclusions

FIG. 13 is a diagram of signaling pathways of leptin and its downstream effectors. obr oligomerization (here only dimerization shown for reasons of clarity) results in phosphorylation and activation of cytoplasmic associated jak2 kinases. These activated jaks phosphorylate tyrosine residues in the cytoplasmic tail of the receptor. Recruitment and activation of secondary signaling molecules allow obr signaling via the jak/stat, niapk, pi3k, anipk, and rotor pathways. Diagram is from Wauman, et al. Front. Endocrinol., Sec. Molecular and Structural Endocrinology Vol. 8 (2017) https://doi.org/10.3389/fendo.2017.00030

The data in the examples show that HDAC6 inhibitors with high levels of penetration of the BBB, especially the hypothalamus, especially inhibitors which block LepRb-HDAC6 interaction in the AgRP neurons in the hypothalamus, are effective in treating leptin-resistant obesity, as well as to increase leptin sensitivity. The critical region of the brain for the inhibitors to be effective is the hypothalamus, where HDAC6 must be inhibited in the Arcuate neurons. Several compounds were tested and it was determined that a central, not peripheral, mechanism of action was involved with the HDAC6 inhibitors that had high levels of penetration through the blood brain barrier and into the hypothalamus. Analysis of hypothalamic gene expressions in fed, starved and obese mice identified correlations with gene expression changes induced by small molecule inhibition of HDAC6 activity, establishing that pharmacological inhibition of HDAC6 activity in the hypothalamus is effective for the treatment of obesity and related disorders. Subsequent studies in high fat diet induced mice treated with HDAC6 isoform-specific inhibitors, ACY738, ACY775, ACY257, and ACY1083, as well as bavrostat, showed these compounds caused significant reduction of food intake and total body fat mass. Effects were greater for the more selective HDAC6 inhibitor ACY775 as compared to ACY738. Significant weight loss was demonstrated by ACY1083, bavarostat and a compound that inhibits HDAC6 in the hypothalamus but has low BBB penetration, ACY257.

Only HDAC6 inhibitors which penetrate the hypothalamus and inhibit HDAC6 therein are effective to treat obesity, especially leptin-resistant obesity. Data demonstrates that a compound, ACY 257, which inhibits HDAC6 in the hypothalamus but does not inhibit HDAC6 in other brain regions such as cerebrum, frontal lobe, temporal lobe, and brainstem, greatly reduces the body weight. The hypothalamus is a very small part of the brain. The hypothalamus has a slightly more permeable blood brain barrier compared to other parts of the brain. The data indicates that inhibitors of HDAC6 which have the ability to preferentially pass through the BBB and into the hypothalamic region, especially the arcuate nucleus, are able to reduce body weight.

Based on these studies, it was determined that small molecule (less than 1000 Da) selective inhibitors of HDAC6 (inhibitors result in a-tubulin acetylation with no impact on histone acetylation), especially inhibitors which block LepRb-HDAC6 interaction, having a high penetration through the BBB of greater than 1 brain/plasma ratio and of the hypothalamus, are most effective for treatment of obesity, including leptin-resistant obesity, via a central nervous system (CNS) mechanism of action. Preferred HDAC6 inhibitors have >0.25, >0.5, or >1 brain or hypothalamus/plasma concentration. More preferably, the HDAC6 inhibitors have >0.25, >0.5, or >1 hypothalamus/plasma concentration. Most preferably, HDAC6 inhibitors which have >0.25, >0.5, or >1 Arcuate Nucleus/plasma concentration. The arcuate nucleus has the highest BBB permeability in the hypothalamus and is around 1/10,000 of the total area of the brain. The results indicate that inhibiting HDAC6 in the AgRP neurons in the arcuate nucleus is sufficient to create the weight loss. If an HDAC6 inhibitor just gets to the arcuate nucleus at concentrations that can inhibit HDAC6 activity, but not to the other regions of the brain, total brain/plasma or hypothalamus/plasma concentrations may be low. However, the arcuate nucleus concentration will be higher than total brain/plasma and hypothalamus/plasma concentrations.

Preferred compounds are ACY775 (brain/plasma ratio 1.26) and ACY738 (brain/plasma ratio of 1.22), ACY257 which has high penetrance into the arctuate nucleus of the hypothalamus, ACY1083, and bavarostat, which has high penetrance of the hypothalamus. Peripherally acting HDAC6 inhibitors such as ricolinostat (ACY1215) with a brain/plasma ratio of 0.01 and tubastatin A with a brain/plasma ratio of 0.18, neither of which have high penetrance into the hypothalamus nor citarinostat (ACY241), which does not show HDAC6 inhibition in the AGP neurons in the hypothalamus, are effective. HDAC6 inhibition was tested by treating cells with a high concentration of HDAC6 inhibitor and analyzing for tubulin acetylation, the established biomarker for HDAC6 inhibition.

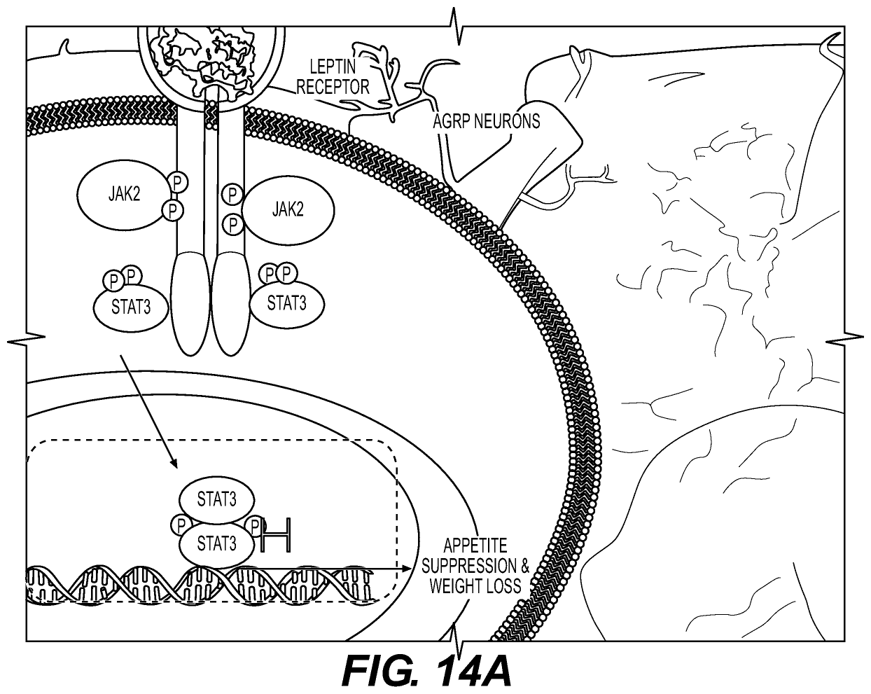

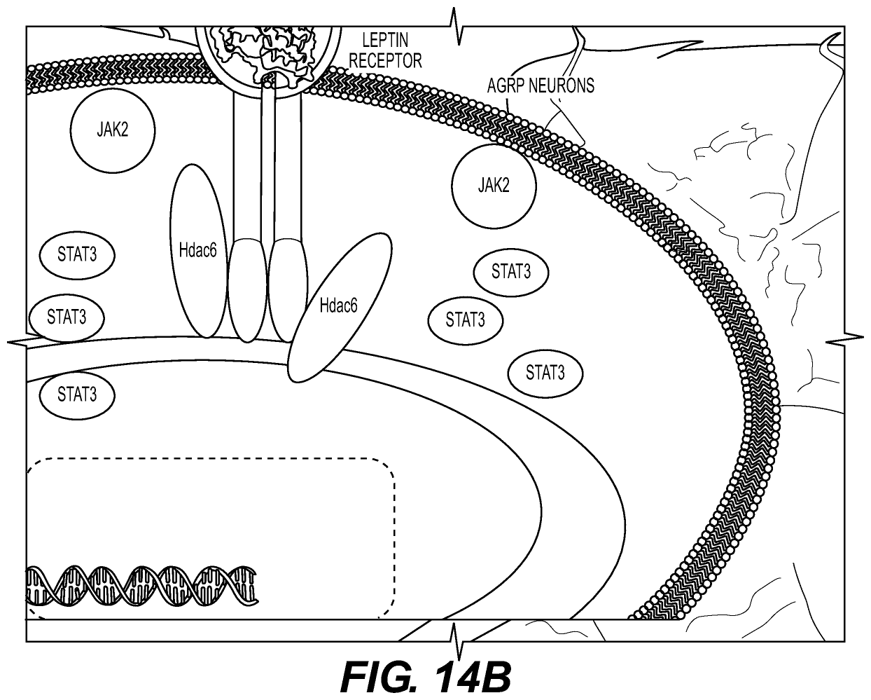

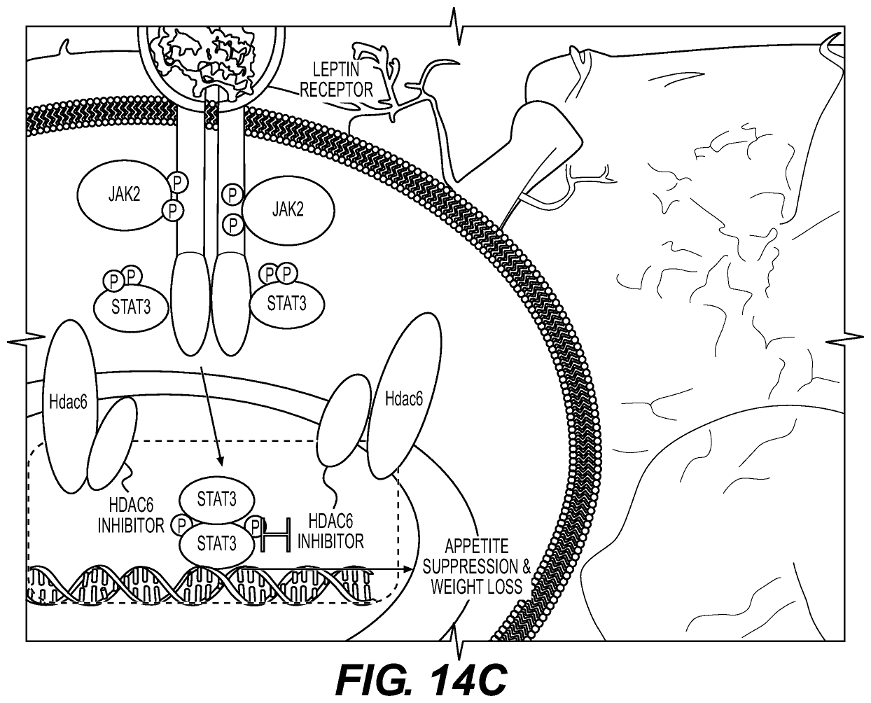

FIGS. 14A-14C are schematics of the sites where the disclosed compounds inhibit or disrupt the HDAC6 or Leptin receptor binding. FIG. 13A is a schematic of binding of Leptin to LepR leads to its tyrosine phosphorylation by Jak2 and subsequently Stat3 phosphorylation and activation. Stat3 plays a crucial role in mediating leptin's appetite suppressing and weight lowering effects. FIG. 13B is a schematic of Hdac6 interaction with LepRb during fasting or obesity reduces LepR, and consequently Stat3 activation. This leads to inhibition of LepR signaling, increased appetite and weight gain. FIG. 13C is a schematic of Hdac6 inhibitors, by blocking association of Hdac6 with LepR, increases LepR activity, suppresses appetite, increases energy expenditure and leads to weight loss.

The selective HDAC6 inhibitors are preferably administered to a mucosal surface, most preferably orally, buccally, or nasally. These can be formulated using known excipients. Formulations may also be formulated for sustained, delayed, and/or pulsatile release to deliver an effective amount of HDAC6 inhibitor to cause weight loss. They are preferably administered once or twice daily. Dosage is based on weight. Typical dosages will be in the range of 25 to 500 mg/day.

The pharmaceutical formulations can be administered to induce weight loss in a pre-obese, obese, or morbidly obese patient, reduce body fat in a pre-obese, obese, or morbidly obese patient, reduce food intake in a pre-obese, obese, or morbidly obese patient, improve glucose homeostasis in a pre-obese, obese, or morbidly obese patient, or combinations thereof. In some cases, a pharmaceutical formulation containing one or more of the HDAC6 inhibitors is administered to a pre-obese, obese, or morbidly obese patient in a therapeutically effective amount to induce weight loss, preferably in a therapeutically effective amount and time of administration to decrease body mass or body fat by at least 10%, more preferably by at least 15%, most preferably by at least 20%, or higher. In some cases, a pharmaceutical formulation containing one or more of the HDAC6 inhibitors is administered to a pre-obese, obese, or morbidly obese patient in a therapeutically effective amount to reduce food intake, appetite, or combinations thereof, preferably in a therapeutically effective amount to reduce average daily food intake (in terms of calories). In some cases, a pharmaceutical formulation containing one or more of the selective HDAC6 inhibitors is administered to a pre-obese, obese, or morbidly obese patient in a therapeutically effective amount to improve glucose homeostasis, preferably in a therapeutically effective amount to reduce average fasting plasma blood glucose. In cases where the pharmaceutical formulations are administered to normalize blood sugar, the formulations are preferably administered in an amount effective to lower blood glucose levels to less than about 180 mg/dL. The formulations can be co-administered with other anti-diabetic therapies, if necessary, to improve glucose homeostasis.

1. A dosage unit formulation inducing weight loss in an obese human having a body mass index of 25 or more comprising an effective amount of a selective HDAC6 inhibitor having a molecular weight of 1000 Da or less and producing a hypothalamus/plasma ratio of more than 0.25, inhibiting HDAC6 in the arcuate AgRP neurons, in pharmaceutically acceptable excipients for administration to a mucosal surface or injection to cause weight loss in an individual having a body mass index of 25 or more.

2. The formulation of claim 1 wherein the selective HDAC6 inhibitor blocks LepRb-HDAC6 interaction in the hypothalamus.

3. The formulation of claim 1 wherein the selective HDAC6 inhibitor with high blood brain or hypothalamus barrier penetration is selected from the group consisting of ACY775, ACY738, ACY257, ACY1083, Bavarostat, prodrugs, analogs and derivatives thereof and produces a hypothalamus/plasma ratio of more than 0.5.

4. The formulation of claim 3 wherein the HDAC6 inhibitor is selected from the group consisting of ACY775, ACY738, ACY257, ACY1083 and Bavarostat and produces a hypothalamus/plasma ratio of more than 1.

5. The formulation of claim 1 wherein the HDAC6 inhibitor induces weight loss in obese but not lean human individuals.

6. The formulation of claim 1 formulated for administration orally, buccally, nasally, pulmonarily, vaginally, or rectally.

7. The formulation of claim 1 for administration by injection into tissue.

8. The formulation of claim 1 providing controlled release of the selective HDAC6 inhibitor.

9. The formulation of claim 1 providing a dosage unit containing one or more of the HDAC6 inhibitors in a therapeutically effective amount when administered to a pre-obese, obese, or morbidly obese leptin resistant human patient to induce weight loss, to reduce food intake, appetite, or combinations thereof, or to improve glucose homeostasis measured as a reduction in the average fasting plasma blood glucose.

10. The formulation of claim 9 providing an effective dosage to cause weight loss in an individual having a body mass index of 30 or more, when administered once per day.

11-19. (canceled)

20. The formulation of claim 1 wherein the HDAC6 inhibitor is ACY775.

21. The formulation of claim 1 wherein the HDAC6 inhibitor is ACY738.

22. The formulation of claim 1 wherein the HDAC6 inhibitor is ACY257.

23. The formulation of claim 1 wherein the HDAC6 inhibitor is ACY1083.

24. The formulation of claim 1 wherein the HDAC6 inhibitor is Bavarostat.
